(19) United States
(12) Patent Application Publication
(10) Pub. No.: US 2012/0150204 A1
(43) Pub. Date: Jun. 14, 2012

(54) IMPLANTABLE SILK PROSTHETIC DEVICE AND USES THEREOF

(75) Inventors: Enrico Mortarino, Hickory, NC (US); Gregory H. Altman, Arlington, MA (US); John Gross, Pasadena, CA (US)

(73) Assignee: ALLERGAN, INC., Irvine, CA (US)

(21) Appl. No.: 13/186,139

(22) Filed: Jul. 19, 2011

Related U.S. Application Data
(63) Continuation-in-part of application No. 12/680,404, filed on Sep. 19, 2011.
(60) Provisional application No. 61/122,520, filed on Dec. 15, 2008.

(51) Int. Cl. A61B 17/03

(52) U.S. Cl. 606/151

(57) ABSTRACT

A method of using a biocompatible surgical silk mesh prosthetic device in body aesthetics and body contouring, the surgical mesh employing a knit pattern that substantially prevents unraveling and preserves the stability of the mesh device, especially when the mesh device is cut. An example prosthetic device employs a knitted mesh including at least two yarns laid in a knit direction and engaging each other to define a plurality of nodes. The at least two yarns include a first yarn and a second yarn extending between and forming loops about two nodes. The second yarn has a higher tension at the two nodes than the first yarn. the second yarn substantially prevents the first yarn from moving at the two nodes and substantially prevents the knitted mesh from unraveling at the nodes.

### CROSS-REFERENCE TO RELATED APPLICATIONS

This United States patent application is a continuation-in-part patent application which claims priority to U.S. utility patent application Ser. No. 12/680,404, filed Mar. 26, 2010, which is a national stage entry of PCT/US09/63717, filed Nov. 9, 2009, claiming priority to U.S. provisional patent application No. 61/122,520, filed Dec. 15, 2008, all of which applications are hereby expressly incorporated by reference herein in their entireties.

### FIELD OF THE INVENTION

The present invention generally relates to a method of using a prosthetic device in body aesthetics and body contouring, and, more particularly, to a method of using a prosthetic device employing a stable knit structure in body aesthetics and body contouring.

### BACKGROUND OF THE INVENTION

Surgical mesh initially used for hernia and abdominal wall defects are now being used for other types of tissue repair, such as rotator cuff repair, pelvic floor dysfunction, and reconstructive or cosmetic surgeries. It is projected that in 2010, there will be more than 8 million hernia procedures, 800,000 rotator cuff repairs, 3 million pelvic prolapse repairs, 600,000 urinary incontinence repairs, and 1.5 million reconstructive or aesthetic plastic surgeries. Most of these procedures will likely employ implantable surgical mesh devices currently on the market, including: Bard Mesh (polypropylene) by C. R. Bard; Dexon (polyglycolic acid) by Synecture/US Surgical; Gore-Tex (polytetraflouroethylene) by W.L. Gore; Prolene (polypropylene), Prolene Soft (polypropylene), Mersilene Mesh (polyester), Gynemesh (polypropylene), Vicryl Knitted Mesh (polyglactin 910), TVT (polypropylene) by Ethicon; Sparc tape (polypropylene) by American Medical Systems; and IVS tape (polypropylene) by TYCO Healthcare International.

Surgical mesh devices are typically biocompatible and may be formed from bioresorbable materials and/or non-bioresorbable materials. For example, polypropylene, polyester, and polytetraflouroethylene (PTFE) are biocompatible and non-bioresorbable, while polyglactin 910 and polyglycolic acid are biocompatible and bioresorbable.

Though current surgical mesh devices may be formed from different materials, they have various similar physical and mechanical characteristics beneficial for tissue repair. However, despite the benefits provided by current surgical mesh devices, their use may be accompanied by a variety of complications. Such complications, for example, may include scar encapsulation and tissue erosion, persistent infection, pain, and difficulties associated with revision surgery. In addition, the use of an absorbable material may result in reoccurrence due to rapid resorption of the implant material and loss of strength.

Although polypropylene monofilament may be a highly regarded material for surgical mesh devices, polypropylene mesh devices can induce intense scar formations and create a chronic foreign body reaction with the formation of a fibrous capsule, even years after implantation. Minor complaints of seromas, discomfort, and decreased wall mobility are frequent and observed in about half of the patients implanted with polypropylene mesh devices. Moreover, polypropylene generally cannot be placed next to the bowel due to the propensity of adhesion formation.

Although the use of multifilament polyester may improve conformity with the abdominal wall, it is also associated with a variety of disadvantages. For example, higher incidences of infection, enterocutaneous fistula formation, and small bowel obstruction have been reported with the use of multifilament polyester compared to other materials. Indeed, the small interstices of the multifilament yarn make it more susceptible to the occurrence of infection, and thus multifilament polyester is not commonly used within the United States.

The use of polytetraflouroethylene (PTFE) may be advantageous in minimizing adhesions to the bowel. However, the host tissue encapsulates the PTFE mesh, resulting in weak in-growth in the abdominal wall and weaker hernia repair. This material, though not a good mesh material on its own, has found its place as an adhesion barrier.

Absorbable materials, such as Vicryl and Dexon, used for hernia repair have the advantage of being placed in direct contact with the bowel without adhesion or fistula formation. A study has observed that Vicryl has comparable burst strength to nonabsorbable mesh at three weeks but is significantly weaker at twelve weeks due to a quick absorption rate. Meanwhile, the same study observed that Dexon has more in-growth at twelve weeks with less absorption of the mesh. The concern with absorbable meshes is that the rate of absorption is variable, possibly leading to hernia recurrence if the proper amount of new tissue is not there to withstand the physiologic stresses placed on the hernia defect.

A significant characteristic of a biomaterial is its porosity, because porosity is the main determinant for tissue reaction. Pore sizes of >500-600 μm permit in-growth of soft tissue; pore sizes of >200-300 μm favor neo-vascularisation and allow mono-morphological restitution of bony defects; pore sizes of <200 μm are considered to be almost watertight, hindering liquid circulation at physiological pressures; and pores of <100 μm only lead to in-growth of single cell types instead of building new tissues. Finally, a pore size of <10 μm hinders any in-growth and increases the chance of infection, sinus tract formation, and encapsulation of the mesh. Bacteria averaging 1 μm in size can hide in the small interstices of the mesh and proliferate while protected from neutrophilic granulocytes averaging 10-15 μm.

Other important physical characteristics for surgical mesh devices include thickness, burst strength, and material stiffness. The thickness of surgical mesh devices vary according to the particular repair procedure. For example, current surgical mesh device hernia, pelvic floor dysfunction, and reconstructive/cosmetic procedures range in thickness from approximately 0.635 mm to 1.1 mm. For rotator cuff repair, a thickness of 0.4 mm to 5 mm is typically employed.

Intra-abdominal pressures of 10-16 N, with a mean distension of 11-32% results in the need for a surgical mesh with a burst strength that can resist the stress of the inner abdomen before healthy tissue comes into being.

Material stiffness is an important mechanical characteristic for surgical mesh, especially when used for pelvic floor dysfunction, because material stiffness has been associated with the likelihood of tissue erosion. Surgical mesh devices formed from TVT, IVS, Mersilene, Prolene, Gynemesh, Sparc tape, for example, currently have an ultimate tensile strength (UTS) that exceeds the forces exerted by intra-abdominal pressures of 10-16N. With the low force in the abdomen, the initial stiffness of the material is an important consideration. Moreover, the stiffness may exhibit non-linear behavior most likely due to changes in the fabric structure, e.g., unraveling of the knit, weave, etc. A surgical mesh device of lesser stiffness may help reduce tissue erosion and may conform to the contours of the body more effectively.

### SUMMARY OF THE INVENTION

In view of the disadvantages of current surgical mesh devices, there continues to be a need for a surgical mesh that is biocompatible and absorbable, has the ability to withstand the physiological stresses placed on the host collagen, and minimizes tissue erosion, fistulas, or adhesions. Thus, embodiments according to aspects of the present invention provide a biocompatible surgical silk mesh prosthetic device for use in soft and hard tissue repair. Examples of soft tissue repair include hernia repair, rotator cuff repair, cosmetic surgery, implementation of a bladder sling, or the like. Examples of hard tissue repair, such as bone repair, involve reconstructive plastic surgery, ortho trauma, or the like.

Advantageously, the open structure of these embodiments allows tissue in-growth while the mesh device degrades at a rate which allows for a smooth transfer of mechanical properties to the new tissue from the silk scaffold. According to a particular aspect of the present invention, embodiments employ a knit pattern, referred to as a “node-lock” design. The “node-lock” design substantially prevents unraveling and preserves the stability of the mesh device, especially when the mesh device is cut.

In a particular embodiment, a prosthetic device includes a knitted mesh including at least two yarns laid in a knit direction and engaging each other to define a plurality of nodes, the at least two yarns including a first yarn and a second yarn extending between and forming loops about two nodes, the second yarn having a higher tension at the two nodes than the first yarn, the second yarn substantially preventing the first yarn from moving at the two nodes and substantially preventing the knitted mesh from unraveling at the nodes.

In an example of this embodiment, the first yarn and the second yarn are formed from different materials. In another example of this embodiment, the first yarn and the second yarn have different diameters. In further embodiments, wherein the first yarn and the second yarn have different elastic properties. In yet a further example of this embodiment, the at least two yarns are formed from silk.

In another example of this embodiment, a first length of the first yarn extends between the two nodes and a second length of the second yarn extends between the two nodes, the first length being greater than the second length. For instance, the first yarn forms an intermediate loop between the two nodes and the second yarn does not form a corresponding intermediate loop between the two nodes. The first length of the first yarn is greater than the second length of the second yarn.

In yet another example of this embodiment, the first yarn is included in a first set of yarns and the second yarn is included in a second set of yarns, the first set of yarns being applied in a first wale direction, each of the first set of yarns forming a first series of loops at each of a plurality of courses for the knitted mesh, the second set of yarns being applied in a second wale direction, the second wale direction being opposite from the first wale direction, each of the second set of yarns forming a second series of loops at every other of the plurality of courses for the knitted mesh, the first set of yarns interlacing with the second set of yarns at the every other course to define the nodes for the knitted mesh, the second set of yarns having a greater tension than the first set of yarns, the difference in tension substantially preventing the knitted mesh from unraveling at the nodes.

In a further example of this embodiment, the first yarn is included in a first set of yarns and the second yarn is included in a second set of yarns, the first set of yarns and the second set of yarns being alternately applied in a wale direction to form staggered loops, the first set of yarns interlacing with the second set of yarns to define the nodes for the knitted mesh, the alternating application of the first set of yarns and the second set of yarns causing the first set of yarns to have different tensions relative to the second set of yarns at the nodes, the difference in tension substantially preventing the knitted mesh from unraveling at the nodes.

In yet a further example of this embodiment, the first yarn is included in a first set of yarns and the second yarn is included in a second set of yarns, the first set of yarns forming a series of jersey loops along each of a first set of courses for a knitted mesh, the second set of yarns forming a second series of alternating tucked loops and jersey loops along each of a second set of courses for the knitted mesh, the second set of courses alternating with the first set of courses, the second set of yarns having a greater tension than the first set of yarns, the tucked loops of the second set of yarns engaging the jersey loops of the first set of yarns to define nodes for the knitted mesh, the tucked loops substantially preventing the knitted mesh from unraveling at the nodes.

In another particular embodiment, a method for making a knitted mesh for a prosthetic device, includes: applying a first set of yarns in a first wale direction on a single needle bed machine, each of the first set of yarns forming a first series of loops at each of a plurality of courses for a knitted mesh; applying a second set of yarns in a second wale direction on the single needle bed machine, the second wale direction being opposite from the first wale direction, each of the second set of yarns forming a second series of loops at every other of the plurality of courses for the knitted mesh; and applying a third set of yarns in every predetermined number of courses for the knitted mesh, the application of the third set of yarns defining openings in the knitted mesh, wherein the first set of yarns interlaces with the second set of yarns at the every other course to define nodes for the knitted mesh, and the second set of yarns has a greater tension than the first set of yarns, the difference in tension substantially preventing the knitted mesh from unraveling at the nodes.

In yet another embodiment, a method for making a knitted mesh for a prosthetic device, includes: applying a first set of yarns to a first needle bed of a double needle bed machine in a wale direction; applying a second set of yarns to a second needle bed of the double needle bed machine in a wale direction; and applying a third set of yarns in every predetermined number of courses for the knitted mesh, the application of the third set of yarns defining openings in the knitted mesh, wherein the first set of yarns and the second set of yarns are alternately applied to form staggered loops at the first needle bed and the second needle bed, respectively, and the first set of yarns interlaces with the second set of yarns to define nodes for the knitted mesh, the alternating application of the first set of yarns and the second set of yarns causing the first set of yarns to have a different tension relative to the second set of yarns at the nodes, the difference in tension substantially preventing the knitted mesh from unraveling at the nodes.

In a further particular embodiment, a method for making a knitted mesh for a prosthetic device, includes: forming, on a flat needle bed machine, a first series of jersey loops along each of a first set of courses for a knitted mesh; and forming, on the flat needle bed machine, a second series of alternating tucked loops and jersey loops along each of a second set of courses for the knitted mesh, the second set of courses alternating with the first set of courses; wherein the second set of courses has a greater tension than the first set of courses, and the tucked loops along the second set of courses engage the jersey loops of the first set of courses and substantially prevents the knitted mesh from unraveling at the tucked loops. In an example of this embodiment, a continuous yarn forms the first set of courses and the second set of courses. In another example of this embodiment, the first set of courses and the second set of courses are formed by different yarns. In yet another example of this embodiment, the first set of courses and the second set of courses are formed by different yarns having different diameters.

Another aspect of the present invention relates to use of the silk surgical mesh of the present invention in various surgical procedures where body aesthetics or body contouring is desired. In such an example, the silk surgical mesh is used in an abdominal area of a patient. The method comprises providing a silk surgical mesh, wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes, and applying the silk surgical mesh in an abdominal area of a patient. The abdominal area comprises an upper abdominal area and a lower abdominal area. For example in an aspect of the present invention, the silk surgical mesh is applied in the lower abdominal area beneath an arcuate line. For example, the silk surgical mesh is applied in the lower abdominal area and in the upper abdominal area. In another example, the method further comprises suturing the silk surgical mesh to the abdominal area.

In another aspect of the present invention, the mesh has at least two areas that differ in at least one mechanical property. In another aspect, the first area supports a single layer fascia in the lower abdomen and the second area supports a double layer fascia in the upper abdomen. For example, the mechanical property is strength, stiffness or stretch. In yet another aspect, the first area has a greater strength, stiffness, or stretch than the second area. In still yet another aspect, the silk surgical mesh is in a shape of a circle, rectangle, square, ellipse, trapezoid, hexagon, or combination thereof. In another aspect, the silk surgical mesh comprises an opening along a centerline of the silk surgical mesh. For example, the opening is in a shape of a circle, rectangle, square, ellipse, trapezoid, or hexagon. In another aspect, the mesh further comprises a slit with the opening along the centerline of the mesh to provide for placement of the silk surgical mesh around an umbilicus. In yet another aspect, the silk surgical mesh has a weft or warp knit construction.

In another aspect of the present invention, the silk surgical mesh is bioresorbable and capable of supporting stress inflicted on an abdominal wall in the abdominal area of the patient. In yet another aspect, the silk surgical mesh is 50 mm to 500 mm in width, 50 mm to 500 mm in length, and/or 0.4 mm to 4 mm in thickness. In still yet another aspect, the silk is formed from Bombyx mori silkworm silk fibroin and the silk is sericin depleted.

Another aspect of the present invention relates to use of the silk surgical mesh of the present invention in an abdominoplasty or a tummy tuck. The method comprises providing a silk surgical mesh having an opening, and placing the silk surgical mesh around an umbilicus of a patient, wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes.

Another aspect of the present invention relates to use of the silk surgical mesh of the present invention in a facial area of a patient. The method comprises providing a silk surgical mesh, and applying the silk surgical mesh in a facial area of a patient wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes.

Another aspect of the present invention relates to use of the silk surgical mesh of the present invention in a pelvic floor repair. The method comprises providing a silk surgical mesh, and applying the silk surgical mesh in a pelvic area of a patient wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes. Another aspect of the present invention relates to use of the silk surgical mesh of the present invention in a hernia repair. The method comprises providing a silk surgical mesh, and applying the silk surgical mesh in an abdominal area of a patient wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes. A method for using a knitted silk fabric in a surgical procedure can comprise the steps of providing a biodegradable, knitted silk fabric, and placing the biodegradable, knitted silk fabric in or over an abdominal area of a patient. A first silk fabric with an upper edge and a lower edge can be placed in the lower abdomen and the first silk fabric can be positioned with a lower edge at the level of the pubic symphsis, and the upper edge at the lower border of the umbilicus. A second silk fabric with an upper edge and a lower edge can also be placed in the lower abdomen by placing the second silk fabric is placed in the supra-umbilcal region. Preferably, the biodegradable (for example 50% biodegraded or bioresorbed by 100 days after placement of the silk fabric in or on a patient, and complete [at least 90%] biodegraded or bioresorbed over a period of about six to about eighteen months) silk fabric used is a biocompatible, non-woven, waft (vertical) knit, multi-filament silk fabric. A woven material is made by weaving. Woven fabrics are classified as to weave or structure according to the manner in which warp and weft cross each other. The three main types of weaves (woven fabrics) are plain, twill, and satin. On the other hand a knitted fabric is generally softer and more supple than a woven fabric because its thread is treated differently. A knit or knitted fabric is made by using needles (such as for example the needles of a single or double bed knit machine) to pull threads up through the preceding threads to thereby make the fabric (explained in more detail supra). All the silk fabrics within the scope of the present invention are knit or knitted (warp or weft) silk fabrics. Woven (weaved) silk fabric, woven textiles and woven fabrics are not within the scope of the present invention. The silk fabric of the present invention can have can have an antibiotic coating.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1A illustrates the technical back of an example mesh produced on a single needle bed warp knitting machine according to aspects of the present invention.

FIG. 1B illustrates the technical front of the example mesh illustrated in FIG. 1A.

FIG. 2 illustrates an example mesh produced on a double needle bed warp knitting machine according to aspects of the present invention.

FIG. 3 illustrates an example mesh produced with single filament silk yarn according to aspects of the present invention.

FIG. 4 illustrates an example mesh produced on a single needle bed warp knitting machine according to aspects of the present invention.

FIG. 5A illustrates an example mesh produced on a double needle bed warp knitting machine, the example mesh having a parallelepiped pore with a section demonstrating a plush design according to aspects of the present invention.

FIG. 5B illustrates an example mesh produced on a double needle bed warp knitting machine, the example mesh having a hexagonal pore according to aspects of the present invention.

FIG. 6 illustrates example narrow mesh fabrics of varying stitch densities incorporating a plush variation according to aspects of the present invention.

FIG. 7 illustrates an example mesh incorporating loop pile according to aspects of the present invention.

FIG. 8 illustrates an example narrow mesh fabric with pore design achieved through variation in the yarn feed rate according to aspects of the present invention.

FIG. 9A illustrates an example collapsed mesh fabric with hexagonal shaped pores according to aspects of the present invention.

FIG. 9B illustrates an example opened mesh fabric with hexagonal shaped pores according to aspects of the present invention.

FIG. 10 illustrates an example of a stable, non-collapsible, hexagonal-shaped porous mesh fabric according to aspects of the present invention.

FIG. 11A illustrates an example of a three-dimensional mesh with the same technical front and technical back according to aspects of the present invention.

FIG. 11B illustrates the 2.55 mm thickness of the example three-dimensional mesh of FIG. 11A.

FIG. 12 illustrates an example of a three-dimensional mesh with a thickness of 3.28 mm according to aspects of the present invention.

FIG. 13A illustrates the technical front of an example non-porous mesh according to aspects of the present invention.

FIG. 13B illustrates the technical back of the example non-porous mesh of FIG. 13A.

FIG. 13C illustrates the 5.87 mm thickness of the example non-porous mesh of FIG. 13A.

FIG. 14A illustrates an example of a three-dimensional mesh with the same technical front and technical back according to aspects of the present invention.

FIG. 14B illustrates the 5.36 mm thickness of the example three-dimensional mesh of FIG. 14A.

FIG. 15A illustrates the technical front of an example three-dimensional mesh fabric according to aspects of the present invention.

FIG. 15B illustrates the technical back of the example three-dimensional mesh fabric of FIG. 15A.

FIG. 16 illustrates an example mesh produced on a double needle bed weft knitting machine demonstrating shaping of the mesh for a breast support application according to aspects of the present invention.

FIG. 17 illustrates another example mesh produced on a double needle bed weft knitting machine demonstrating shaping of the mesh for a breast support application according to aspects of the present invention.

FIG. 18 illustrates yet another example mesh produced on a double needle bed weft knitting machine demonstrating shaping of the mesh for a breast support application according to aspects of the present invention.

FIG. 19 illustrates a further mesh produced on a double needle bed weft knitting machine demonstrating shaping of the mesh for a breast support application according to aspects of the present invention.

FIG. 20 illustrates another example mesh produced on a double needle bed weft knitting machine demonstrating shaping of the mesh for a breast support application according to aspects of the present invention.

FIG. 21A illustrates a full-thickness rat abdominal defect created using a custom designed 1-cm stainless steel punch, the defect appearing oval in shape due to body wall tension applied.

FIG. 21B illustrates a 4 cm×4 cm example implant centered on top of the open defect of FIG. 21A, and held in place with single interrupted polypropylene sutures (arrow) through the implant and muscle.

FIG. 21C illustrates an explanted specimen 94 days post implantation as shown in FIG. 21B.

FIG. 21D illustrates ball burst testing performed with a 1-cm diameter ball pushed through the defect site reinforced with the mesh according to aspects of the present invention.

FIG. 22 illustrates an example pattern layout for a single needle bed mesh according to aspects of the present invention.

FIG. 23 illustrates an example pattern layout for a single needle bed mesh according to aspects of the present invention.

FIG. 24 illustrates an example pattern layout for a single needle bed mesh according to aspects of the present invention.

FIG. 25 illustrates an example pattern layout for the single needle bed mesh according to aspects of the present invention.

FIG. 26 illustrates an example pattern layout of the double needle bed mesh according to aspects of the present invention.

FIG. 27 illustrates an example pattern layout for the double needle bed weft knitting machine according to aspects of the present invention.

FIG. 28A is a photograph of a pattern layout for a silk-based mesh design in accordance with aspects of the present invention.

FIGS. 28B and 28C illustrate an example pattern layout for the mesh design of FIG. 28A including all pattern and ground bars according to aspects of the present invention.

FIGS. 28D and 28E illustrate an example pattern layout for a double needle bed mesh or scaffold according to aspects of the present invention from FIG. 28B for ground bar #4.

FIGS. 28F and 28G illustrate an example pattern layout for a double needle bed mesh or scaffold according to aspects of the present invention from FIG. 28B for pattern bar #5.

FIGS. 28H and 28I illustrate an example pattern layout for a double needle bed mesh or scaffold according to aspects of the present invention from FIG. 28B for ground bar #7.

FIG. 28J illustrates an example pattern simulation for a double needle bed mesh demonstrated in FIG. 28B according to aspects of the present invention.

FIG. 29A is a photograph of a pattern layout for a silk-based mesh in accordance with aspects of the present invention.

FIGS. 29B and 29C illustrate an example pattern layout for the mesh design of FIG. 29A including all pattern and ground bars according to aspects of the present invention.

FIGS. 29D and 29E are enlarged views of the example pattern layout and ground bars of FIG. 29B.

FIGS. 30A and 30B illustrate an example pattern layout for a double needle bed mesh or scaffold according to aspects of the present invention from FIG. 29B for ground bar #4.

FIGS. 30C and 30D are enlarged views of the example pattern layout and ground bars of FIG. 29B.

FIGS. 31A and 31B illustrate an example pattern layout for a double needle bed mesh or scaffold according to aspects of the present invention from FIG. 29B for pattern bar #5.

FIGS. 31C and 31D are enlarged views of the example pattern layout and ground bars of FIG. 29B.

FIGS. 32A and 32B illustrate an example pattern layout for a double needle bed mesh according to aspects of the present invention from FIG. 29B for ground bar #7.

FIGS. 32C and 32D are enlarged views of the example pattern layout and ground bars of FIG. 29B.

FIG. 33 illustrates an example pattern simulation for a double needle bed mesh demonstrated in FIG. 29B according to aspects of the present invention.

FIG. 34A is a photograph of a pattern layout for a silk-based mesh in accordance with aspects of the present invention.

FIGS. 34B and 34C illustrate an example pattern layout for a silk-based mesh design of FIG. 34A for use as a mesh in accordance with aspects of the present invention including all pattern and ground bars according to aspects of the present invention.

FIGS. 34D and 34E are enlarged views of the example pattern layout and ground bars of FIG. 34B.

FIGS. 35A and 35B illustrate an example pattern layout for a double needle bed mesh according to aspects of the present invention from FIG. 34B for ground bar #2.

FIGS. 35C and 35D are enlarged views of the example pattern layout and ground bars of FIG. 34B.

FIGS. 36A and 36B illustrate an example pattern layout for a double needle bed mesh according to aspects of the present invention from FIG. 34B for pattern bar #4.

FIGS. 36C and 36D are enlarged views of the example pattern layout and ground bars of FIG. 34B.

FIGS. 37A and 37B illustrate an example pattern layout for a double needle bed mesh according to aspects of the present invention from FIG. 34B for pattern bar #5.

FIGS. 37C and 37D are enlarged views of the example pattern layout and ground bars of FIG. 34B.

FIGS. 38A and 38B illustrate an example pattern layout for a double needle bed mesh according to aspects of the present invention from FIG. 34B for ground bar #7.

FIGS. 38C and 38D are enlarged views of the example pattern layout and ground bars of FIG. 34B.

FIG. 39 illustrates an example pattern simulation for a double needle bed mesh demonstrated in FIG. 34B according to aspects of the present invention.

FIG. 40A is a photograph of a pattern layout for a silk-based mesh in accordance with aspects of the present invention.

FIGS. 40B and 40C illustrate an example pattern layout for the silk-based mesh design of FIG. 40A in accordance with the present invention including all pattern and ground bars according to aspects of the present invention.

FIGS. 40D and 40E are enlarged views of the example pattern layout and ground bars of FIG. 40B.

FIG. 41A and FIG. 41B illustrate an example pattern layout for a double needle bed mesh according to aspects of the present invention from FIG. 40B for ground bar #4.

FIGS. 41C and 41D are enlarged views of the example pattern layout and ground bars of FIG. 40B.

FIGS. 42A and 42B illustrate an example pattern layout for a double needle bed mesh according to aspects of the present invention from FIG. 40B for pattern bar #5.

FIGS. 42C and 42D are enlarged views of the example pattern layout and ground bars of FIG. 40B.

FIGS. 43A and 43B illustrate an example pattern layout for a double needle bed mesh according to aspects of the present invention from FIG. 40B for ground bar #7.

FIGS. 43C and 43D are enlarged views of the example pattern layout and ground bars of FIG. 40B.

FIG. 44 illustrates an example pattern simulation for a double needle bed mesh demonstrated in FIG. 40B according to aspects of the present invention.

FIG. 45A is a photograph of a pattern layout for a silk-based mesh in accordance with aspects of the present invention.

FIGS. 45B and 45C illustrate an example pattern layout for the silk-based mesh design of FIG. 45A in accordance with the present invention including all pattern and ground bars according to aspects of the present invention.

FIGS. 45D and 45E are enlarged views of the example pattern layout and ground bars of FIG. 45B.

FIGS. 46A and 46B illustrate an example pattern layout for a double needle bed mesh or scaffold according to aspects of the present invention from FIG. 45B for ground bar #4.

FIGS. 46C and 46D are enlarged views of the example pattern layout and ground bars of FIG. 45B.

FIGS. 47A and 47B illustrate an example pattern layout for a double needle bed mesh or scaffold according to aspects of the present invention from FIG. 45B for pattern bar #5.

FIGS. 47C and 47D are enlarged views of the example pattern layout and ground bars of FIG. 45B.

FIGS. 48A and 48B illustrate an example pattern layout for a double needle bed mesh according to aspects of the present invention from FIG. 45B for ground bar #7.

FIGS. 48C and 48D are enlarged views of the example pattern layout and ground bars of FIG. 45B.

FIG. 49 illustrates an example pattern simulation for a double needle bed mesh demonstrated in FIG. 45B according to aspects of the present invention.

FIG. 50A is a photograph of a pattern layout for a silk-based mesh in accordance with aspects of the present invention.

FIGS. 50B and 50C illustrate an example pattern layout for the silk-based mesh design of FIG. 50A in accordance with the present invention including all pattern and ground bars according to aspects of the present invention.

FIGS. 50D and 50E are enlarged views of the example pattern layout and ground bars of FIG. 50B.

FIGS. 51A and 51B illustrate an example pattern layout for a double needle bed mesh according to aspects of the present invention from FIG. 50B for ground bar #4.

FIGS. 51C and 51D are enlarged views of the example pattern layout and ground bars of FIG. 50B.

FIGS. 52A and 52B illustrate an example pattern layout for a double needle bed mesh according to aspects of the present invention from FIG. 50B for pattern bar #5.

FIGS. 52C and 52D are enlarged views of the example pattern layout and ground bars of FIG. 50B.

FIGS. 53A and 53B illustrate an example pattern layout for a double needle bed mesh according to aspects of the present invention from FIG. 50B for ground bar #7.

FIGS. 53C and 53D are enlarged views of the example pattern layout and ground bars of FIG. 50B.

FIG. 54 illustrates an example pattern simulation for a double needle bed mesh demonstrated in FIG. 50B according to aspects of the present invention.

FIG. 55 illustrates a surgical mesh imposed over an area of reinforcement.

FIG. 56 illustrates a mesh with two areas having different mechanical properties.

FIG. 57A illustrates a mesh formed with equal degree for its angles.

FIG. 57B illustrates a mesh formed with equal degree for its angles with a round opening.

FIG. 58A illustrates a mesh formed with different degree for its angles.

FIG. 58B illustrates a mesh formed with different degree for its angles with a square opening.

FIG. 58C illustrates a mesh formed with different degree for its angles with an elliptical opening.

FIG. 59 illustrates a mesh with an opening for the umbilicus.

FIG. 60A illustrates a mesh in small size.

FIG. 60B illustrates a mesh in medium size.

FIG. 60C illustrates a mesh in large size.

### DETAILED DESCRIPTION

All the silk fabrics within the scope of the present invention are knit silk fabrics intended for implantation in a human body. The word “knit” is synonymous with the word “knitted”, so that a knit silk fabric is the same as a knitted silk fabric. The silk fabrics within the scope of the present invention can be warp knit or they can weft knit silk fabrics. Preferably, the silk fabric of the present invention is a biocompatible, warp knit, multi-filament silk fabric. Woven (weaved) silk fabric, woven textiles and woven fabrics are not within the scope of the present invention. A woven material or fabric is made by weaving, which is a process that does not use needles, and results in a fabric with different characteristics. In particular, a woven fabric is made by a non-needle process using multiple yarns that interlace each other at right angles to form a structure wherein one set of yarn is parallel to the direction of fabric formation. On the other hand, a knit fabric is made by using needles (such as for example the needles of a single or double bed knit machine) to pull threads (yarn) up through the preceding thread formed into a loop by the needle, to thereby making the knit fabric (explained in more detail supra). In particular, a knitted fabric is made using needles to have a fabric with one or multiple yarn intermeshing (also referred as interloping). Additionally, non-woven fabrics are also not within the scope of the present invention. Non-woven (also refer to as bonded) fabrics are formed by having multiple fibers cohered together chemically or physically, without use of needles.

Embodiments according to aspects of the present invention provide a biocompatible surgical silk mesh device for use in soft or hard tissue repair. Examples of soft tissue repair include hernia repair, rotator cuff repair, cosmetic surgery, implementation of a bladder sling, or the like. Examples of hard tissue repair, such as bone repair, involve reconstructive plastic surgery, ortho trauma, or the like.

Advantageously, the open structure of these embodiments allows tissue in-growth while the mesh bioresorbs at a rate which allows for a smooth transfer of mechanical properties to the new tissue from the silk scaffold. Furthermore, embodiments employ a knit pattern that substantially prevents unraveling, especially when the mesh device is cut. In particular, embodiments may preserve the stability of the mesh device by employing a knit pattern that takes advantage of variations in tension between at least two yarns laid in a knit direction. For example, a first yarn and a second yarn may be laid in a knit direction to form “nodes” for a mesh device. The knit direction for the at least two yarns, for example, may be vertical during warp knitting or horizontal during weft knitting. The nodes of a mesh device, also known as intermesh loops, refer to intersections in the mesh device where the two yarns form a loop around a knitting needle. In some embodiments, the first yarn is applied to include greater slack than the second yarn, so that, when a load is applied to the mesh device, the first yarn is under a lower tension than the second device. A load that places the at least two yarns under tension may result, for example, when the mesh device is sutured or if there is pulling on the mesh device. The slack in the first yarn causes the first yarn to be effectively larger in diameter than the second yarn, so that the first yarn experiences greater frictional contact with the second yarn at a node and cannot move, or is “locked,” relative to the second yarn. Accordingly, this particular knit design may be referred to as a “node-lock” design.

In general, node-lock designs according to aspects of the present invention employ at least two yarns under different tensions, where a higher tension yarn restricts a lower tension yarn at the mesh nodes. To achieve variations in tension between yarns, other node-lock designs may vary the yarn diameter, the yarn materials, the yarn elastic properties, and/or the knit pattern. For example, the knit pattern described previously applies yarns in varying lengths to create slack in some yarns so that they experience less tension. Because the lower tension yarn is restricted by the higher tension yarn, node-lock designs substantially prevent unraveling of the mesh when the mesh is cut. As such, the embodiments allow the mesh device to be cut to any shape or size while maintaining the stability of the mesh device. In addition, node-lock designs provide a stability that makes it easy to pass the mesh device through a cannula for laparoscopic or arthroscopic surgeries without damaging the material.

Although the node-lock design may employ a variety of polymer materials, a mesh device using silk according to aspects of the present invention can bioresorb at a rate sufficient to allow tissue in-growth while slowly transferring the load-bearing responsibility to the native tissue. Particular embodiments may be formed from Bombyx mori silkworm silk fibroin. The raw silk fibers have a natural globular protein coating known as sericin, which may have antigenic properties and must be depleted before implantation. Accordingly, the yarn is taken through a depletion process. The depletion of sericin is further described, for example, by Gregory H. Altman et al., “Silk matrix for tissue engineered anterior cruciate ligaments,” Biomaterials 23 (2002), pp. 4131-4141, the contents of which are incorporated herein by reference. As a result, the silk material used in the device embodiments contains substantially no sensitizing agents, in so far as can be measured or predicted with standardized biomaterials test methods.

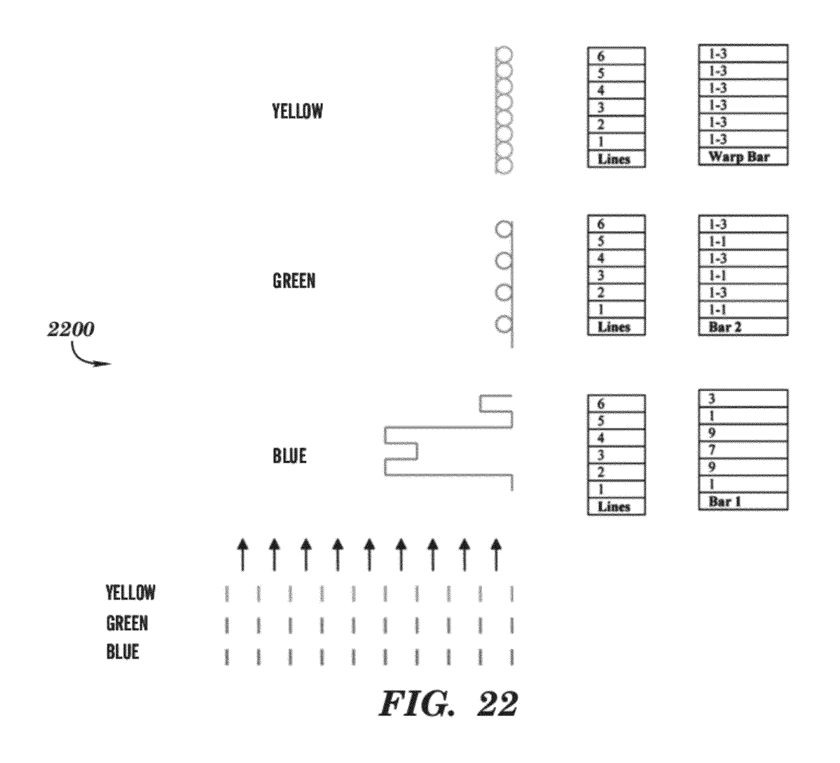

A surgical mesh device according to aspects of the present invention may be created on a single needle bed Comez Acotronic/600-F or a Comez 410 ACO by the use of three movements as shown in the pattern layout 2200 in FIG. 22: two movements in the wale direction, the vertical direction within the fabric, and one in the course direction, the horizontal direction of the fabric. The movements in the wale direction go in opposing directions; a first yarn moving in one direction loops every course while the second yarn moving in the opposite direction loops every other course. The yarns follow a repeated pattern of 3-1 and 1-1/1-3 on a 20 gauge knitting machine, using only half of the needles available on the needle bed. The interlacing of the loops within the fabric allow for one yarn to become under more tension than the other under stress, locking it around the less tensioned yarn; keeping the fabric from unraveling when cut. The other movement within the fabric occurs in every few courses creating the openings in the mesh. These yarns follow a pattern of 1-9/9-7/7-9/9-1/1-3/3-1. These yarns create tension within the fabric when under stress, locking the yarns in the fabric; preventing the fabric from unraveling.

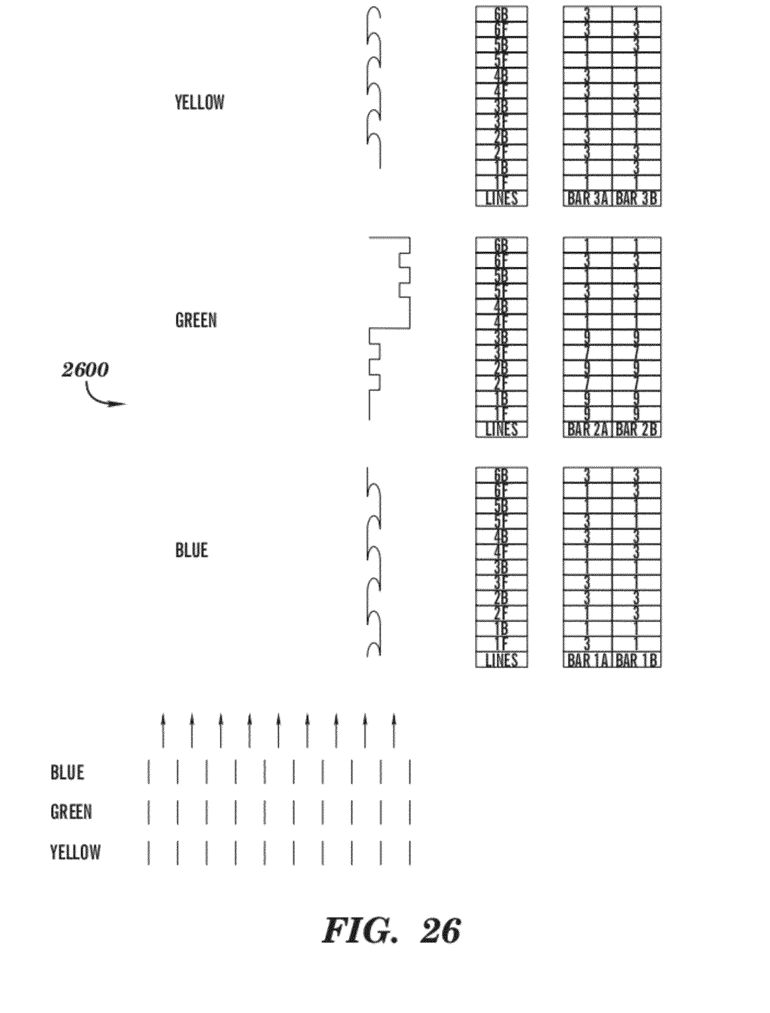

A surgical mesh device according to aspects of the present invention may be created on a double needle bed Comez DNB/EL-800-8B knitting machine by the use of three movements as shown in the pattern layout 2600 in FIG. 26: two movements in the wale direction and one in the course direction. The two movements in the wale direction occur on separate needle beds with alternate yarns; loops that occur in every course movement are staggered within the repeat. The yarns follow a repeated pattern of 3-1/1-1/1-3/3-3 and 1-1/1-3/3-3/3-1. The third movement happens with the yarn that traverses the width of the fabric. The yarn follows the pattern 9-9/9-9/7-7/9-9/7-7/9-9/1-1/1-1/3-3/1-1/3-3/1-1. This fabric is also made at half gauge on a 20 gauge knitting machine and prevents unraveling due to the tension created between the yarns when stressed. The repeat the yarn follows within the pattern is illustrated in FIG. 26.

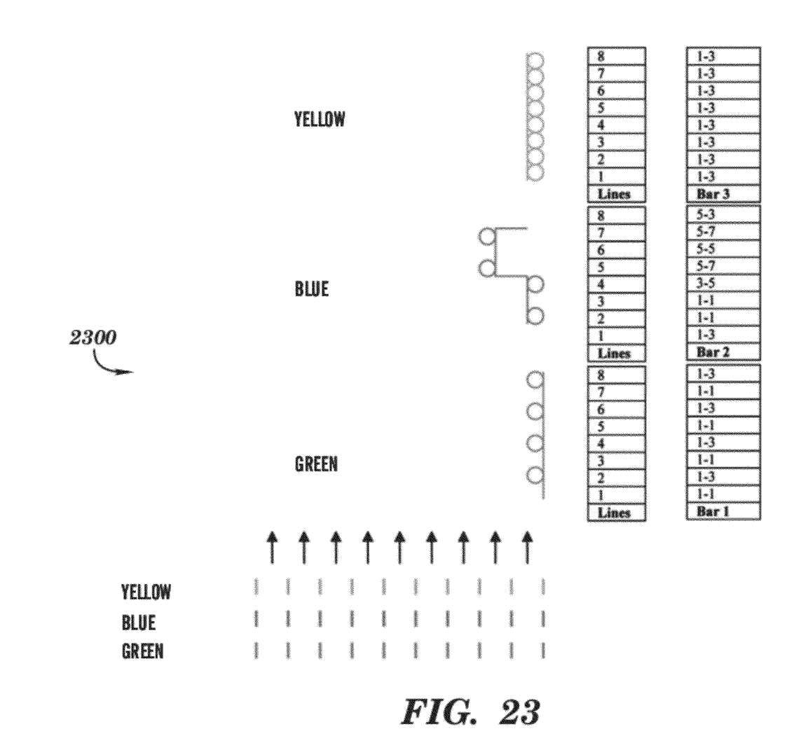

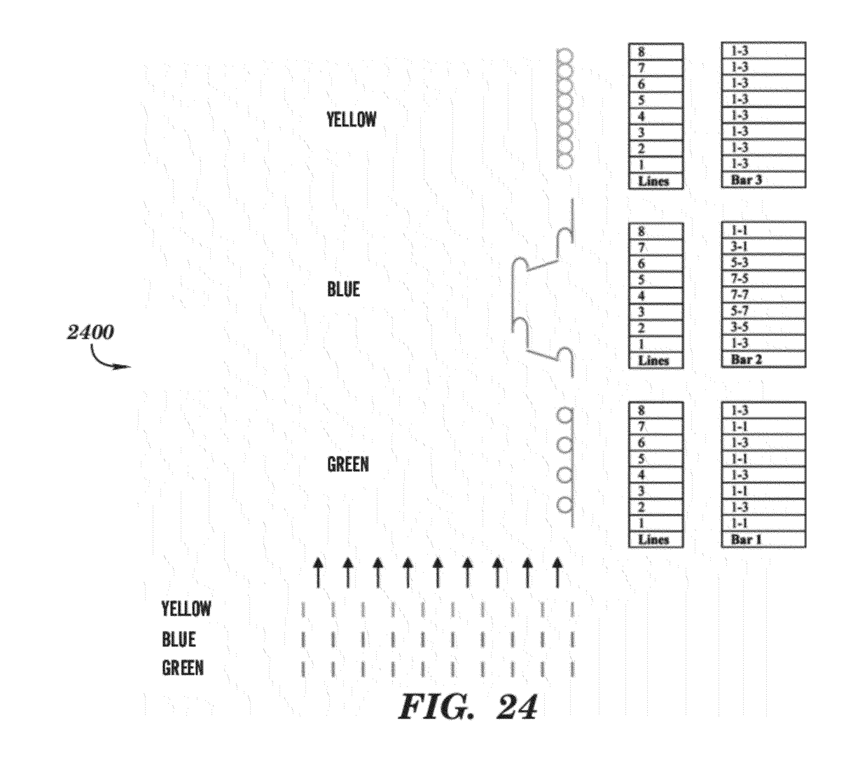

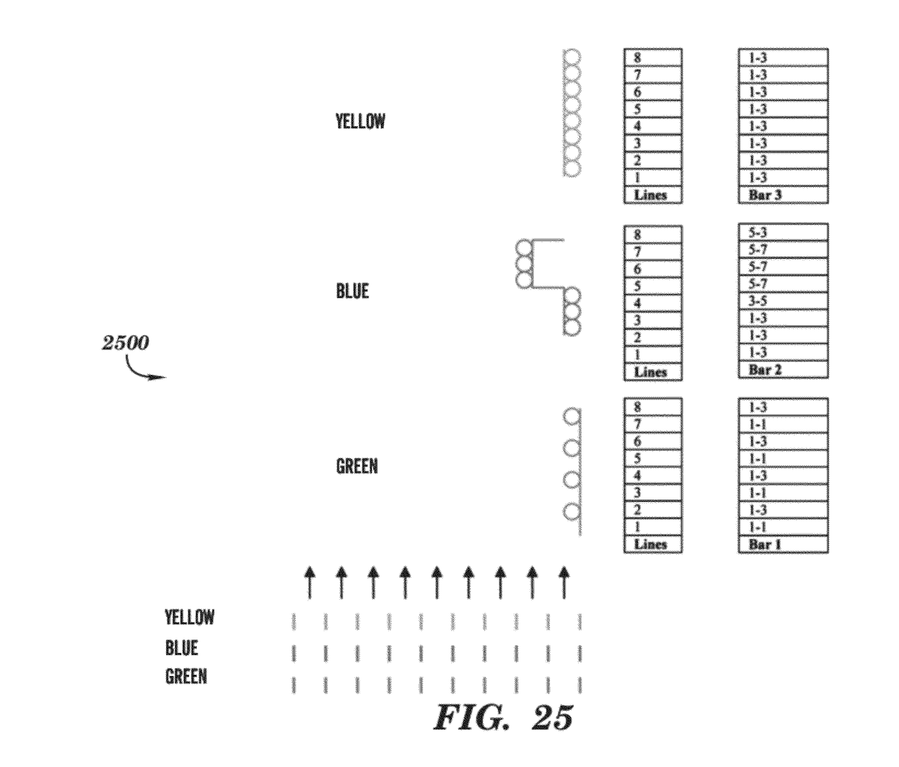

According to the pattern layouts 2300, 2400, and 2500 illustrated in FIGS. 23, 24 and 25, respectively, variations of the surgical mesh pattern are demonstrated for the Single Needle Bed including knitting with an added warp bar in place of using a weft bar insertion. These variations include knitting with the node lock yarns while moving it perpendicularly to one or more wales. These variations may include, but are not limited to, knitting either an open or closed chain stitch in either all or alternating courses. Utilizing a third warp bar, as opposed to a weft bar insertion can also be applied to the double needle warp knitting machine.

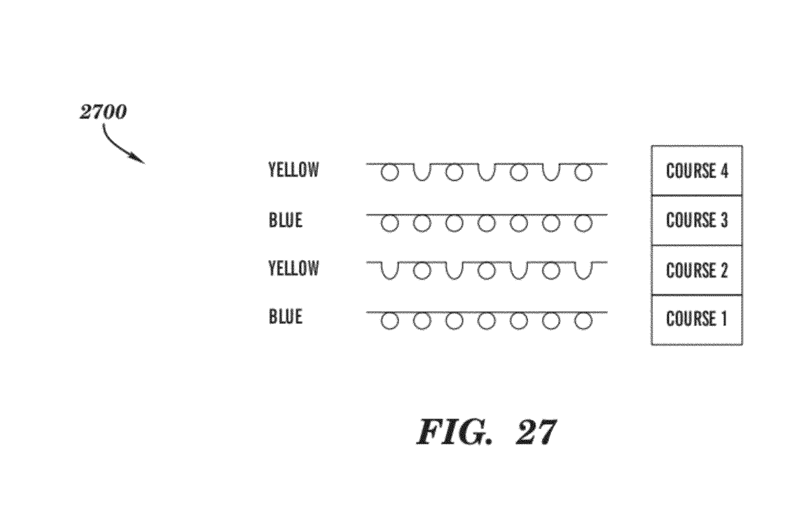

A surgical mesh device according to aspects of the present invention may be formed on the Shima Seiki flat needle bed machine as shown in the pattern layout 2700 in FIG. 27. This knit includes a continuous yarn or at least two different yarn sizes, one of which could be, though not limited to a different material. The knitted mesh would be formed by a regular jersey knit on the first row with loops formed by either a continuous yarn or a yarn of a certain yarn size, while the loops in the second row are formed by tucked loops that occur alternately with jersey knit loops of the same continuous yarn or with a yarn of a different size. The mesh would be shaped during knitting by use of increasing or decreasing stitches; a fashioning technique.

In embodiments employing silk yarn, the silk yarn may be twisted from yarn made by 20-22 denier raw silk fibers approximately 40 to 60 μm in diameter. Preferably, raw silk fibers ranging from 10 to 30 denier may be employed; however any fiber diameters that will allow the device to provide sufficient strength to the intended area are acceptable. Advantageously, a constant yarn size may maximize the uniformity of the surgical mesh mechanical properties, e.g. stiffness, elongation, etc., physical and/or biological properties. However, the yarn size may be varied in sections of the surgical mesh in order to achieve different mechanical, physical and/or biological characteristics in the preferred surgical mesh locations. Factors that may influence the size of the yarn include, but are not limited to: ultimate tensile strength (UTS); yield strength, i.e. the point at which yarn is permanently deformed; percent elongation; fatigue and dynamic laxity (creep); bioresorption rate; and transfer of cell/nutrients into and out of the mesh. The knit pattern layouts 2200, 2300, 2400, 2500, and 2600 illustrated in FIGS. 22-26, respectively, may be knitted to any width limited by the knitting machine width and could be knitted with any of the gauges available with the various crochet machine or warp knitting machine. TABLE 2 outlines the fabric widths that may be achieved using different numbers of needles on different gauge machines. It is understood that the dimensions in TABLE 1 are approximate due to the shrink factor which depends on stitch design, stitch density, and yarn size used.

TABLE 1

| Gauge | Needle Count | Knitting Width |
|---|---|---|
| 48 | 2-5,656 | 0.53-2,997.68 mm |
| 24 | 2-2,826 | 1.06-2,995.56 mm |
| 20 | 2-2,358 | 1.27-2,994.66 mm |
| 18 | 2-2,123 | 1.41-2,993.43 mm |
| 16 | 2-1,882 | 1.59-2,992.38 mm |
| 14 | 2-1,653 | 1.81-2,991.93 mm |
| 12 | 2-1,411 | 2.12-2,991.32 mm |
| 10 | 2-1,177 | 2.54-2,989.58 mm |
| 5 | 2-586 | 5.08-2,976.88 mm |

Embodiments of a prosthetic device according to the present invention may be knitted on a fine gauge crochet knitting machine. A non-limiting list of crochet machines capable of manufacturing the surgical mesh according to aspects of the present invention are provided by: Changde Textile Machinery Co., Ltd.; Comez; China Textile Machinery Co., Ltd.; Huibang Machine; Jakkob Muller AG; Jingwei Textile Machinery Co., Ltd.; Zhejiang Jingyi Textile Machinery Co., Ltd.; Dongguan Kyang the Delicate Machine Co., Ltd.; Karl Mayer; Sanfang Machine; Sino Techfull; Suzhou Huilong Textile Machinary Co., Ltd.; Taiwan Giu Chun Ind. Co., Ltd.; Zhangjiagang Victor Textile; Liba; Lucas; Muller Frick; and Texma.

Embodiments of a prosthetic device according to the present invention may be knitted on a fine gauge warp knitting machine. A non-limiting list of warp knitting machines capable of manufacturing the surgical mesh according to aspects of the present invention are provided by: Comez; Diba; Jingwei Textile Machinery; Liba; Lucas; Karl Mayer; Muller Frick; Runyuan Warp Knitting; Taiwan Giu Chun Ind.; Fujian Xingang Textile Machinery; and Yuejian Group.

Embodiments of a prosthetic device according to the present invention may be knitted on a fine gauge flat bed knitting machine. A non-limiting list of flat bed machines capable of manufacturing the surgical mesh according to aspects of the present invention are provided by: Around Star; Boosan; Cixing Textile Machine; Fengshen; Flying Tiger Machinary; Fujian Hongqi; G & P; Gorteks; Jinlong; JP; Jy Leh; Kauo Heng Co., Ltd.; Matsuya; Nan Sing Machinery Limited; Nantong Sansi Instrument; Shima Seiki; Nantong Tianyuan; and Ningbo Yuren Knitting.

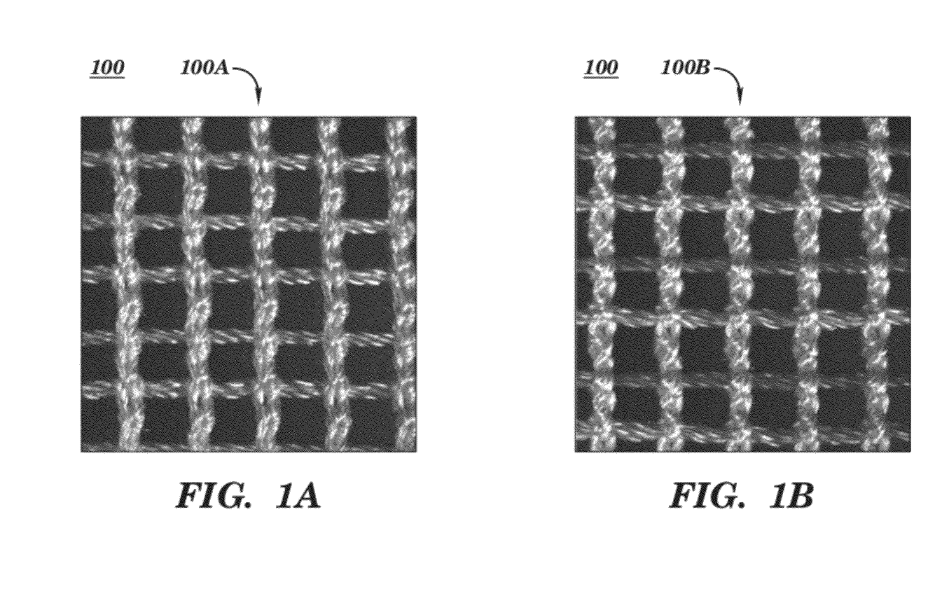

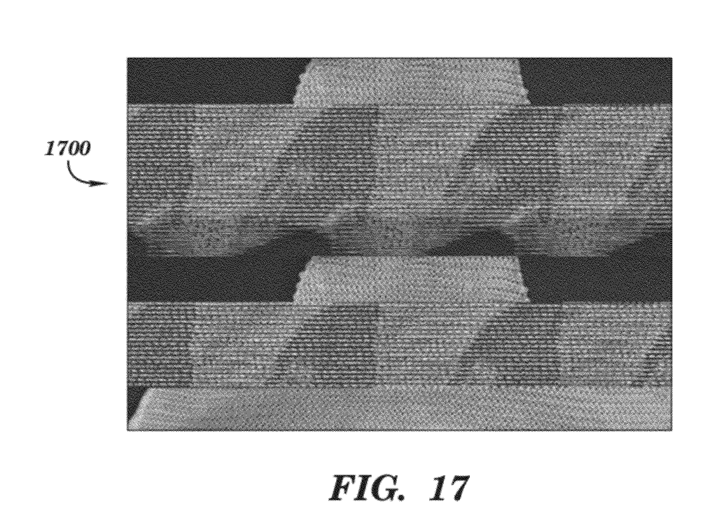

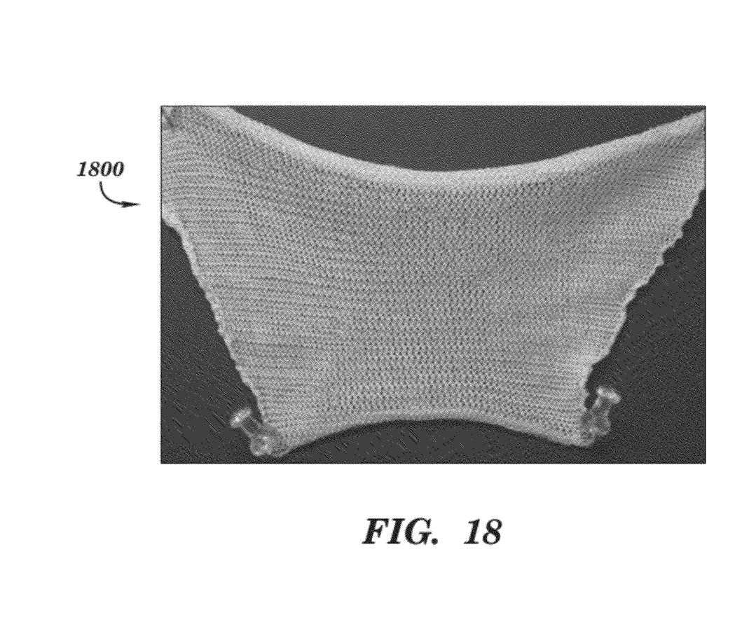

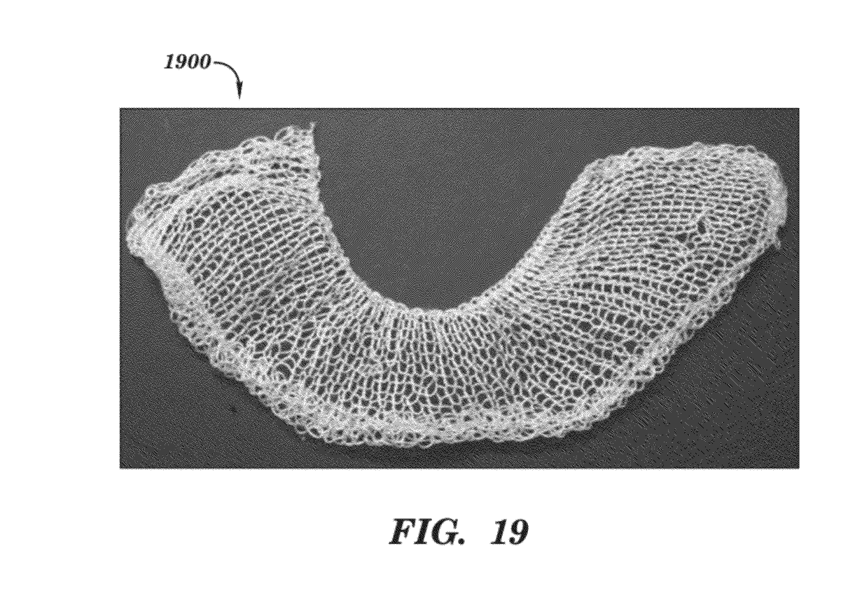

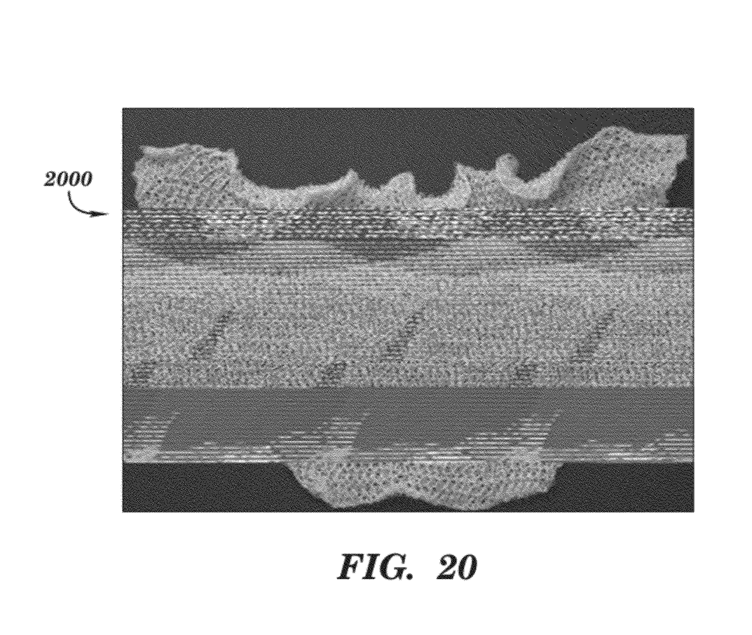

FIGS. 1-20 illustrate example meshes produced according to aspects of the present invention. Referring to FIGS. 1A and B, an example mesh 100 is produced on a single needle bed warp knitting machine according to aspects of the present invention. FIG. 1A shows the technical back 100A of the mesh 100, and FIG. 1B shows the technical front 100B of the mesh 100.

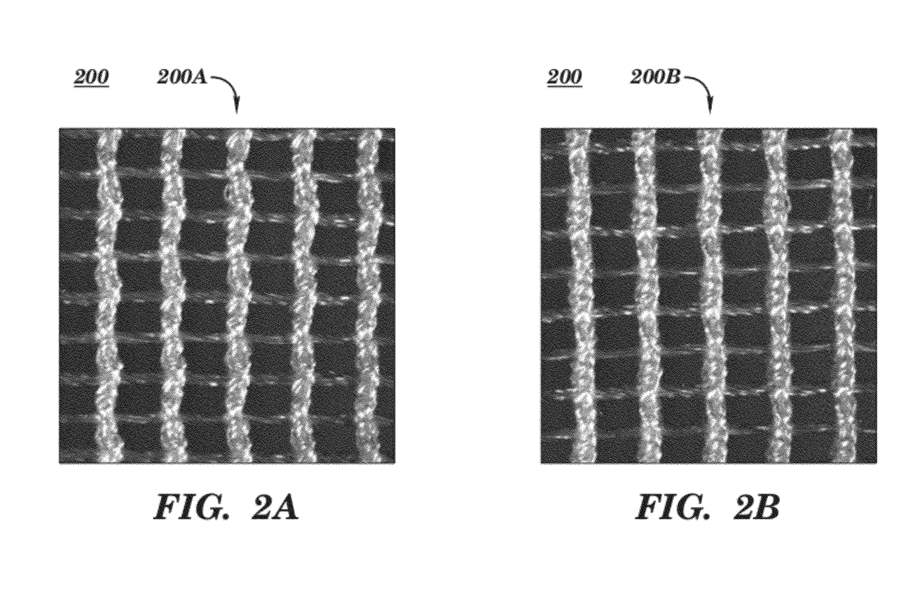

Referring to FIGS. 2A and B, an example mesh 200 is produced on a double needle bed warp knitting machine according to aspects of the present invention. FIG. 2A shows the technical front 200A of the mesh 200, and FIG. 2B shows the technical back 200B of the mesh 200.

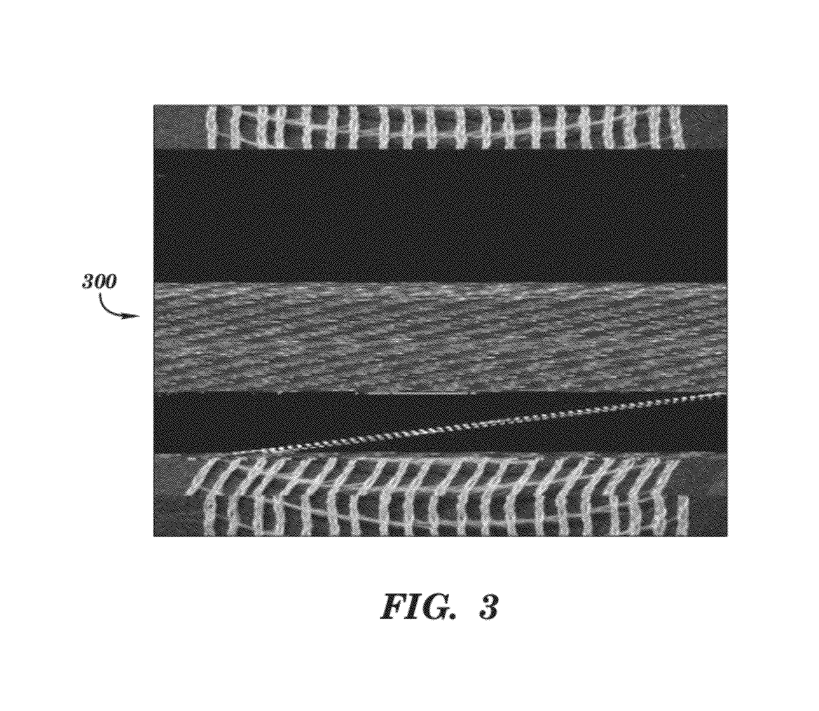

FIG. 3 illustrates an example mesh 300 produced with single filament silk yarn according to aspects of the present invention.

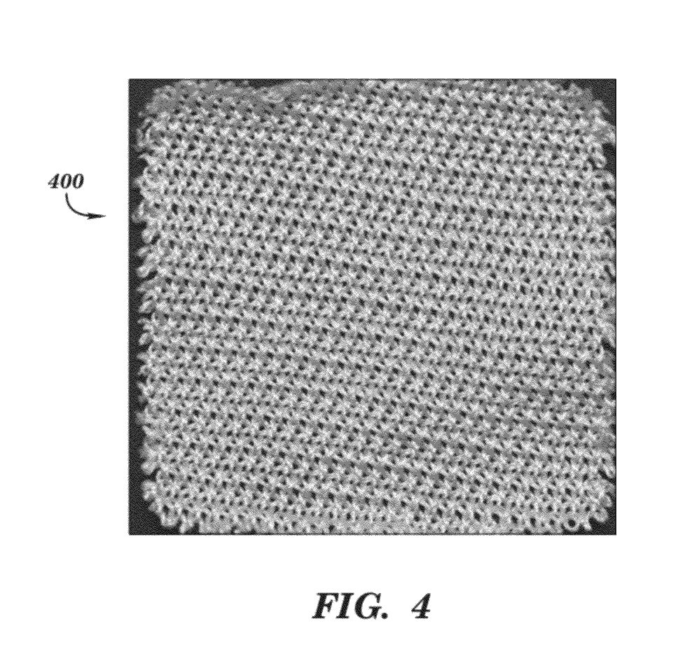

FIG. 4 shows an example mesh 400 produced on a single needle bed warp knitting machine according to aspects of the present invention.

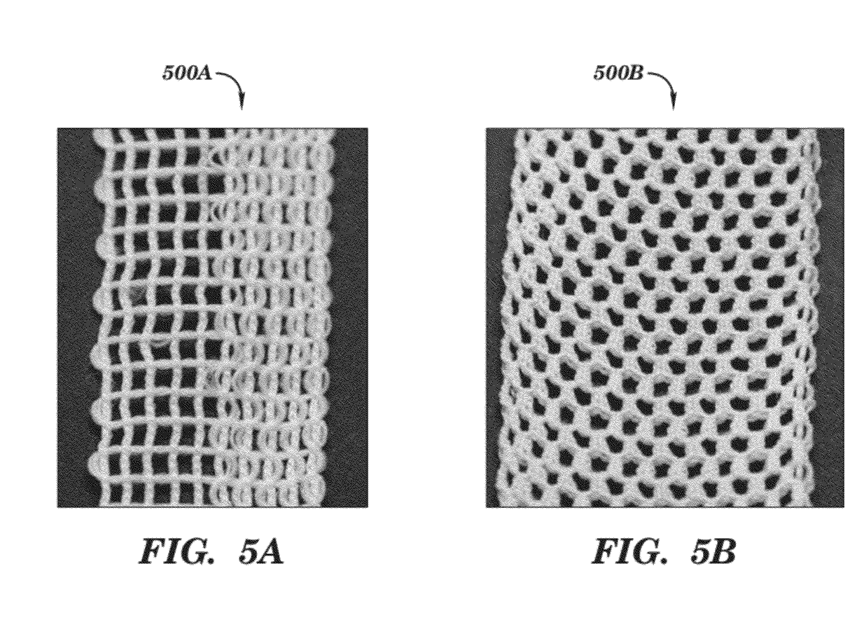

FIG. 5A illustrates an example mesh 500A produced on a double needle bed warp knitting machine. The mesh 500A has a parallelepiped pore with a section demonstrating a plush design according to aspects of the present invention. Meanwhile, FIG. 5B illustrates an example mesh 500B produced on a double needle bed warp knitting machine. The example mesh 500B has a hexagonal pore according to aspects of the present invention.

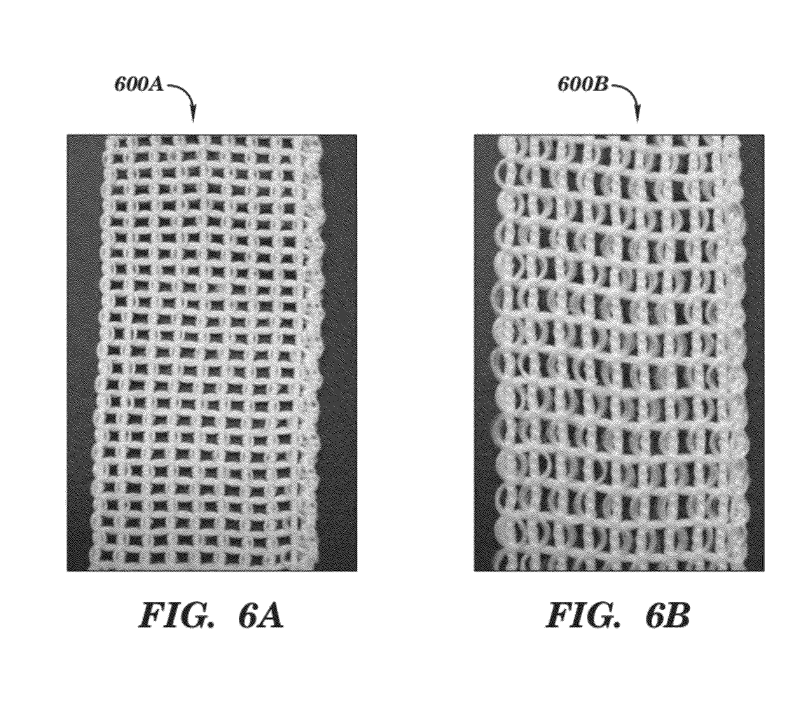

FIGS. 6A and B illustrate example narrow mesh fabrics 600A and 600B according to aspects of the present invention. The mesh fabrics 600A and 600B have varying stitch densities incorporating a plush variation.

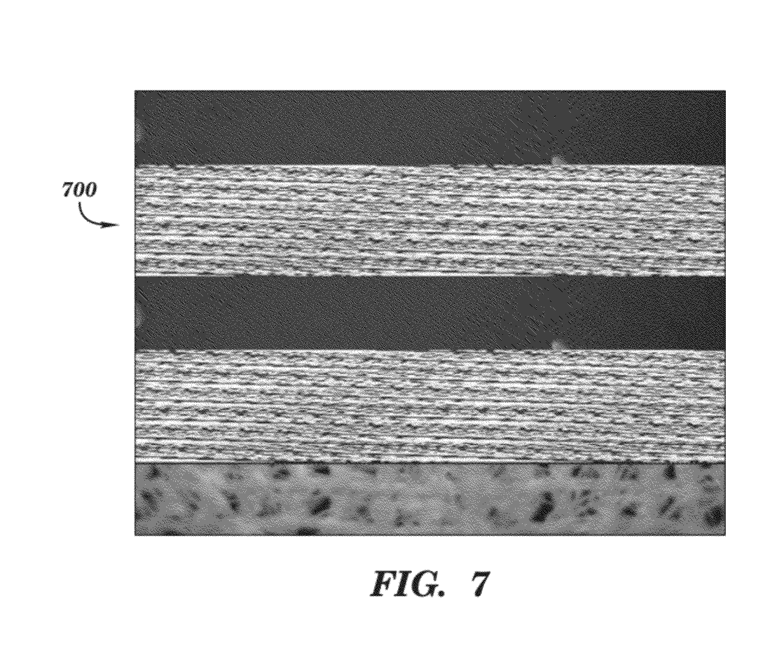

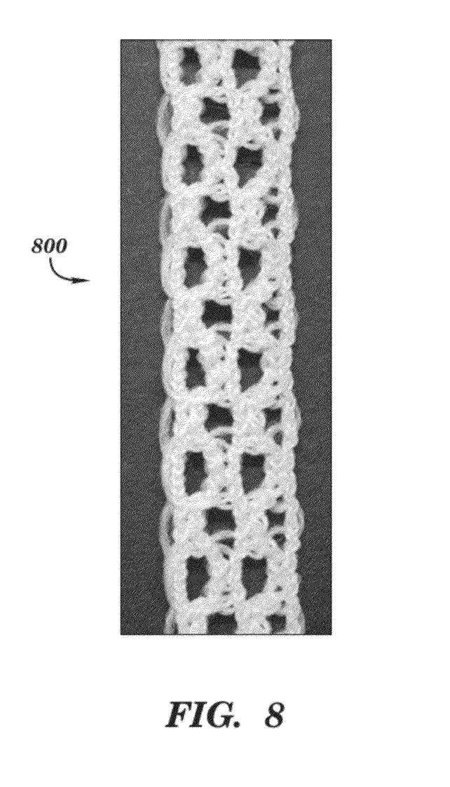

Referring to FIG. 7, an example mesh 700 incorporates loop pile according to aspects of the present invention. FIG. 8 illustrates an example narrow mesh fabric 800 with pore design achieved through variation in the yarn feed rate according to aspects of the present invention.

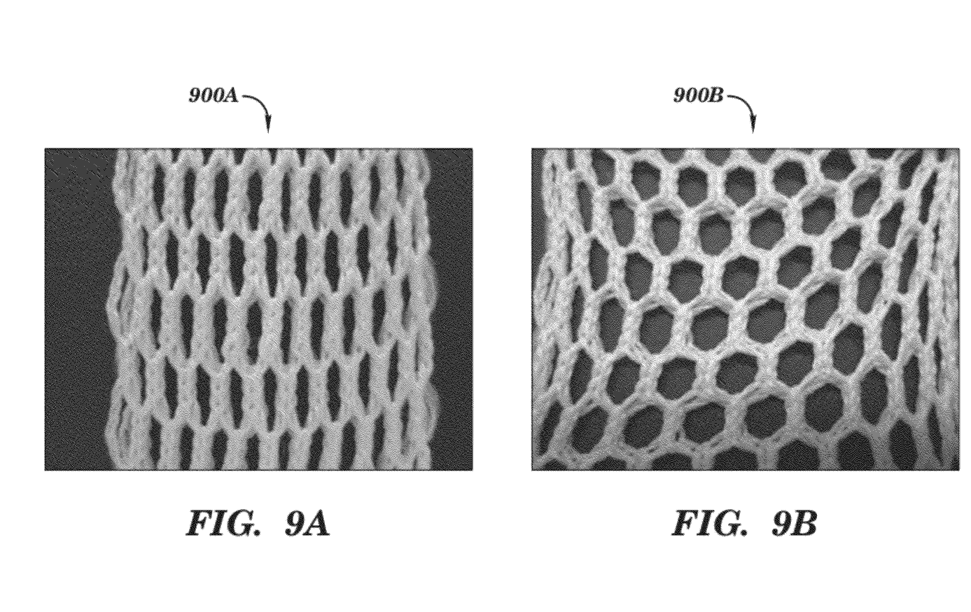

FIG. 9A illustrates an example collapsed mesh fabric 900A with hexagonal-shaped pores according to aspects of the present invention. Meanwhile, FIG. 9B illustrates an example opened mesh fabric 900B with hexagonal shaped pores according to aspects of the present invention.

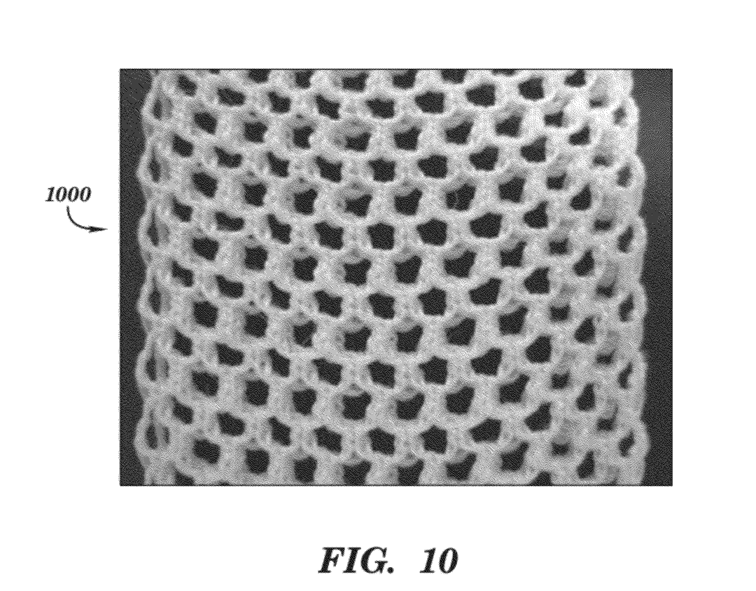

As shown in FIG. 10, an example of a stable, non-collapsible mesh fabric 1000 includes hexagonal-shaped pores according to aspects of the present invention.

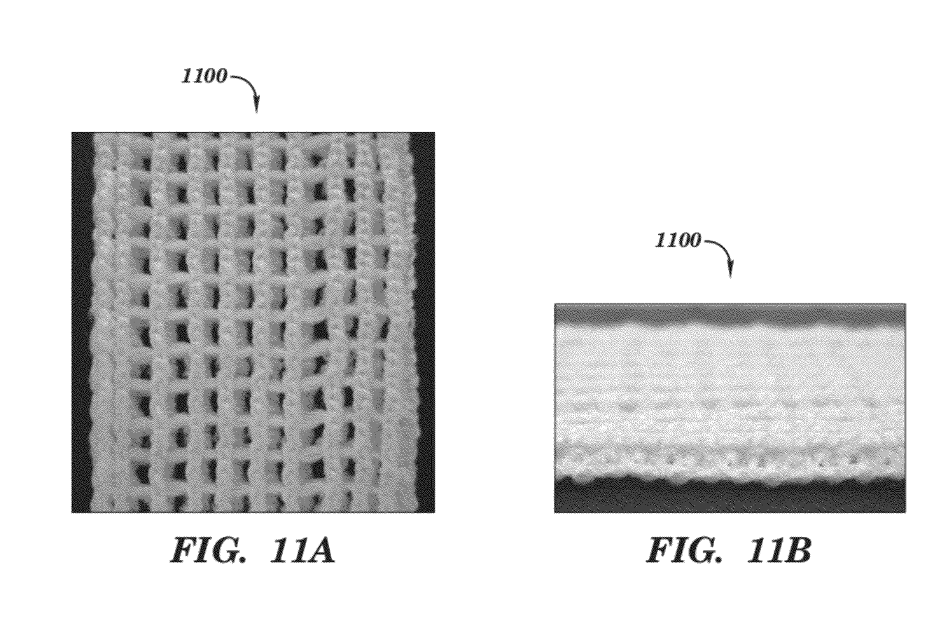

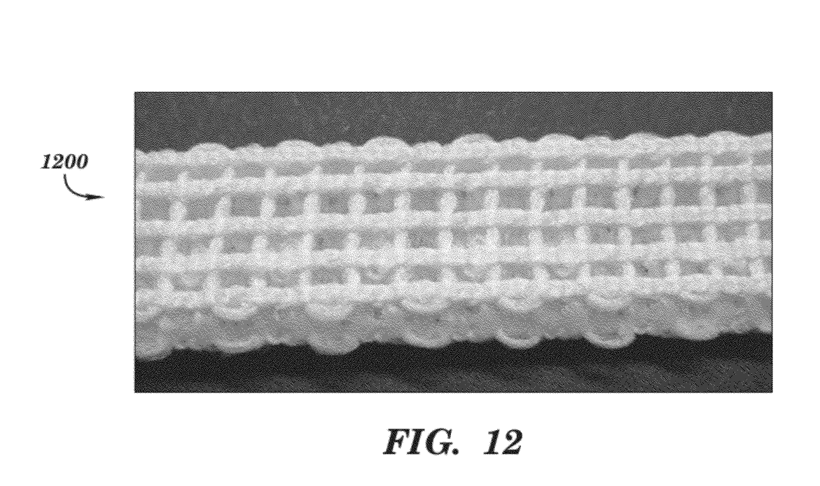

FIG. 11A illustrate an example three-dimensional mesh 1100 with the same technical front and technical back according to aspects of the present invention. FIG. 11B illustrates the 2.55 mm thickness of the three-dimensional mesh 1100. FIG. 12 illustrates another example three-dimensional mesh 1200 with a thickness of 3.28 mm according to aspects of the present invention.

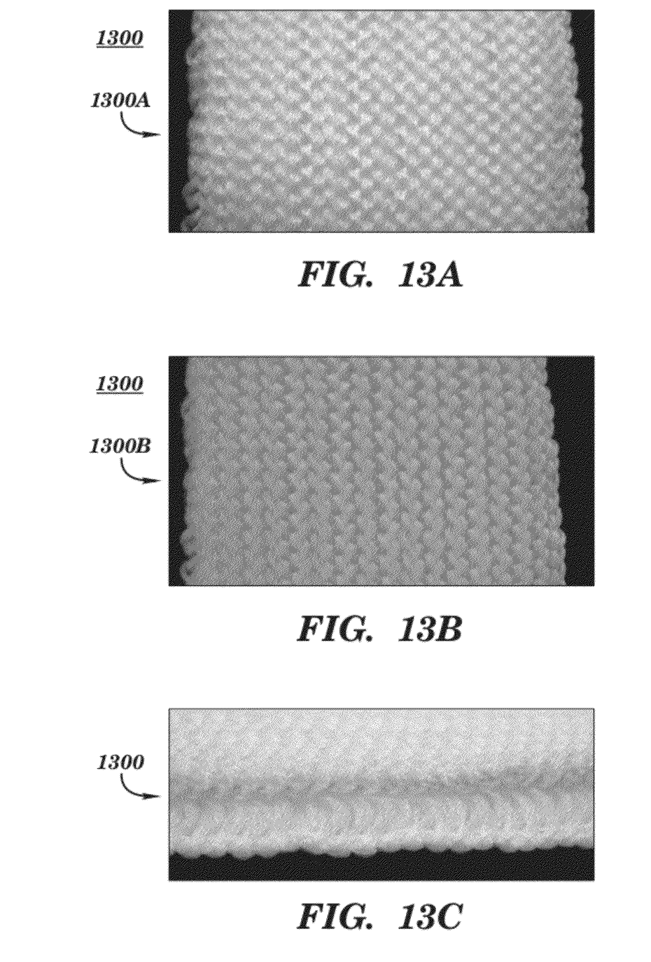

FIGS. 13A-C illustrate an example non-porous mesh 1300 according to aspects of the present invention. FIG. 13A shows the technical front 1300A of the non-porous mesh 1300. FIG. 13B shows the technical back 1300B of the non-porous mesh 1300. FIG. 13C shows that non-porous mesh 1300 has a thickness of 5.87 mm.

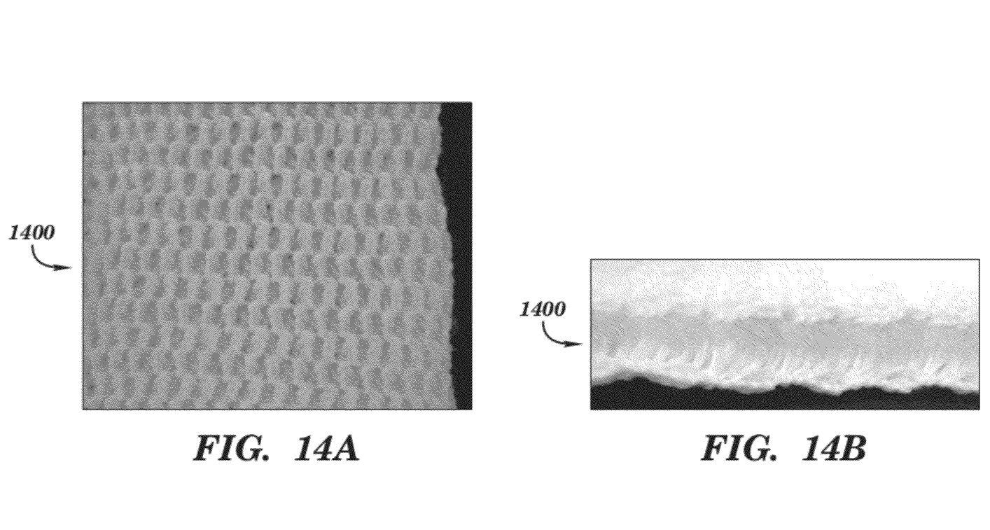

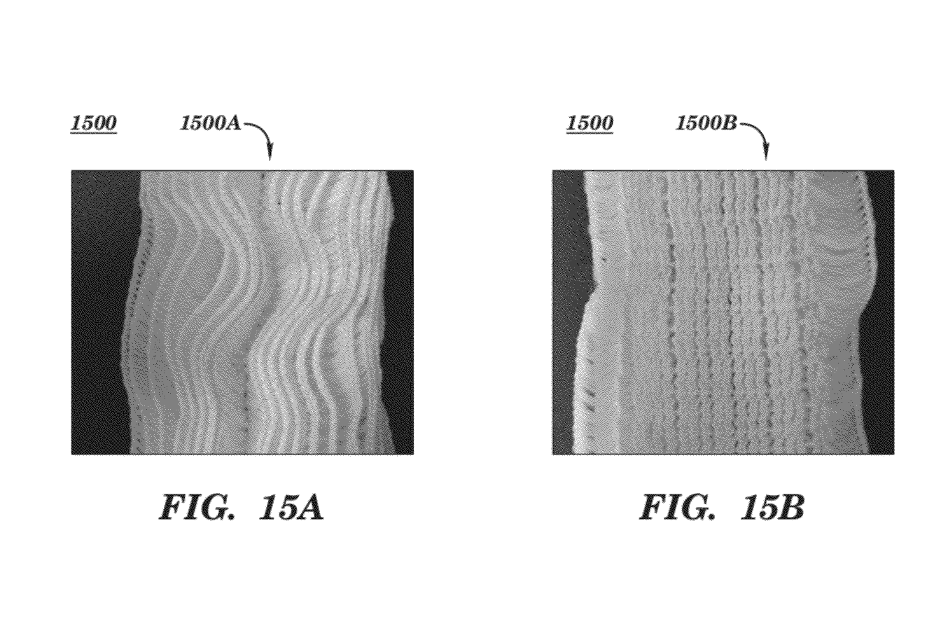

FIG. 14A illustrates an example three-dimensional mesh 1400 with the same technical front and technical back according to aspects of the present invention. FIG. 14B shows that the three-dimensional mesh 1400 has a thickness of approximately 5.36 mm. FIGS. 15A and B illustrate another example three-dimensional mesh fabric 1500 according to aspects of the present invention. FIG. 15A shows the technical front 1500A of the fabric 1500, and FIG. 15B illustrates the technical back 1500B of the fabric 1500.

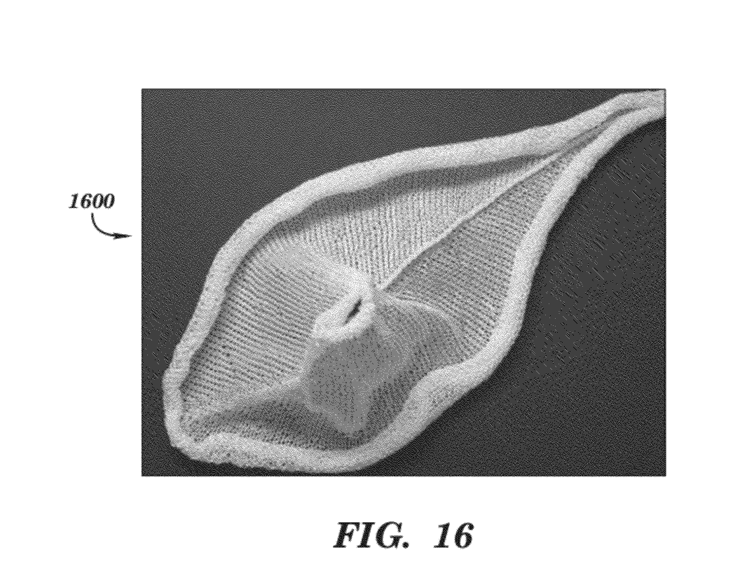

FIGS. 16-20 illustrate respective example meshes 1600, 1700, 1800, 1900, and 2000 that are produced on a double needle bed weft knitting machine. The meshes 1600, 1700, 1800, 1900, and 2000 demonstrate shaping of a mesh for a breast support application according to aspects of the present invention.

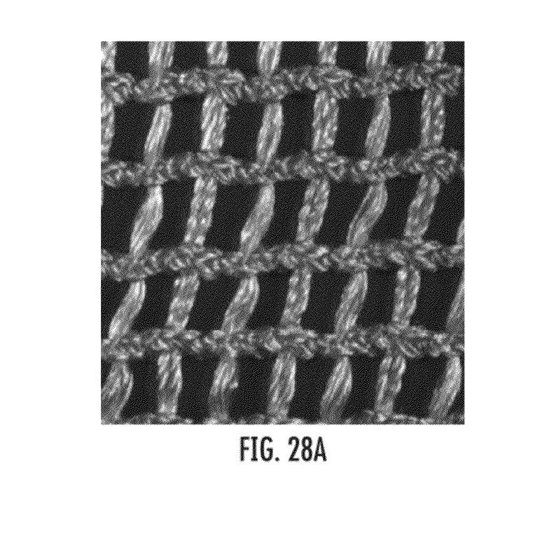

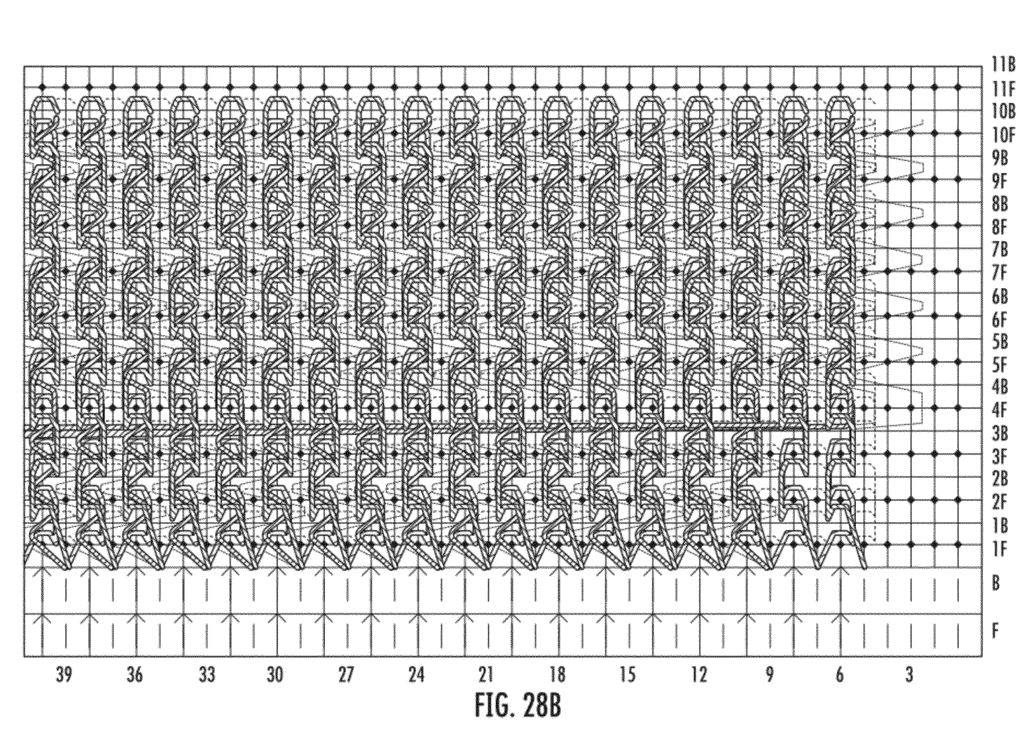

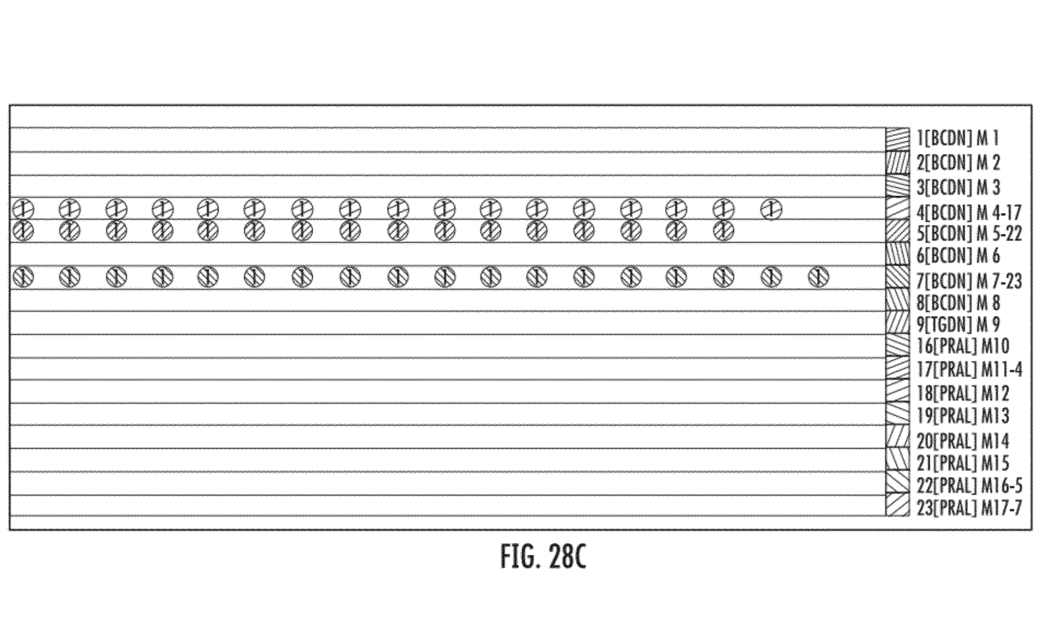

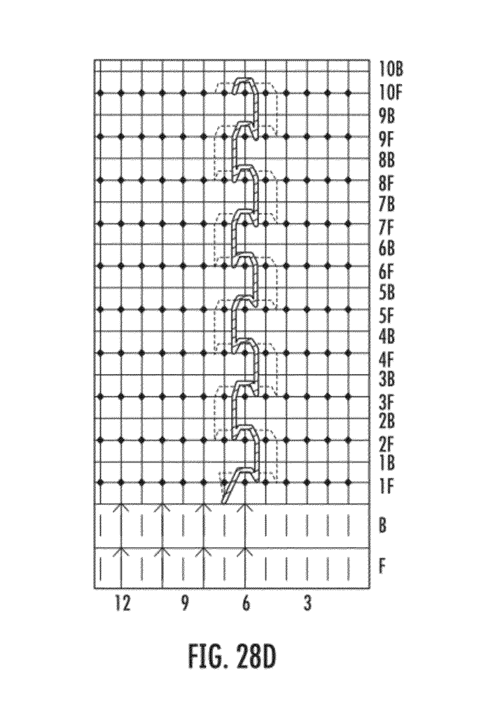

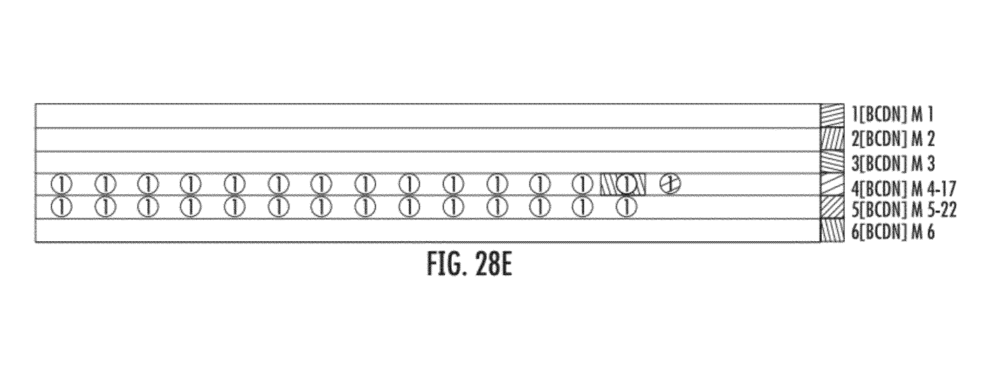

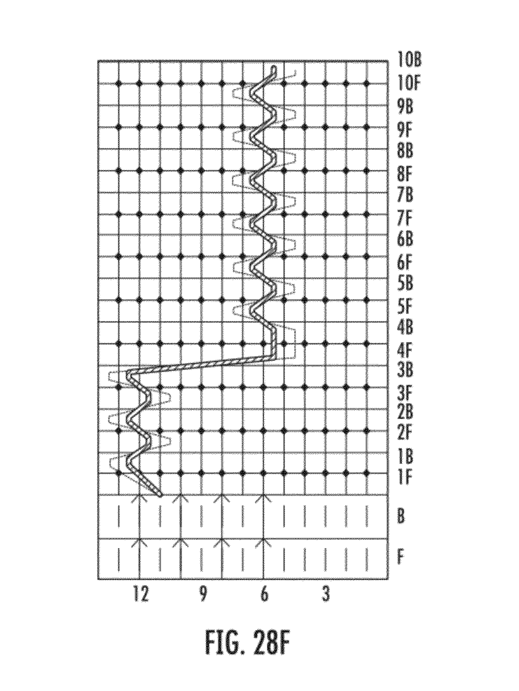

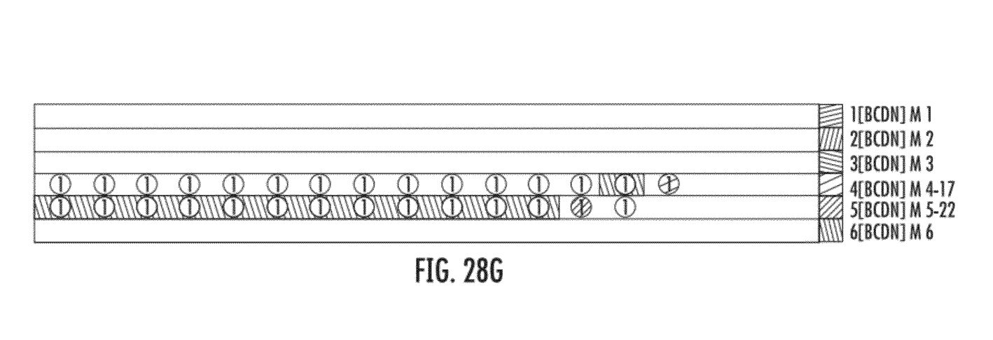

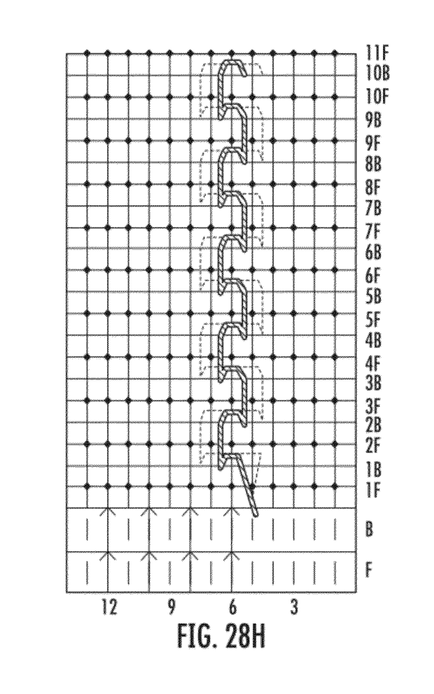

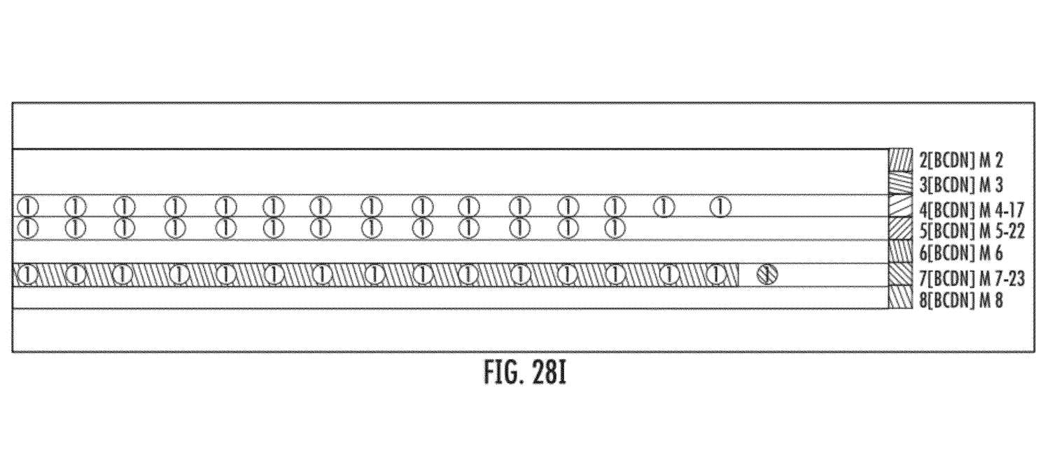

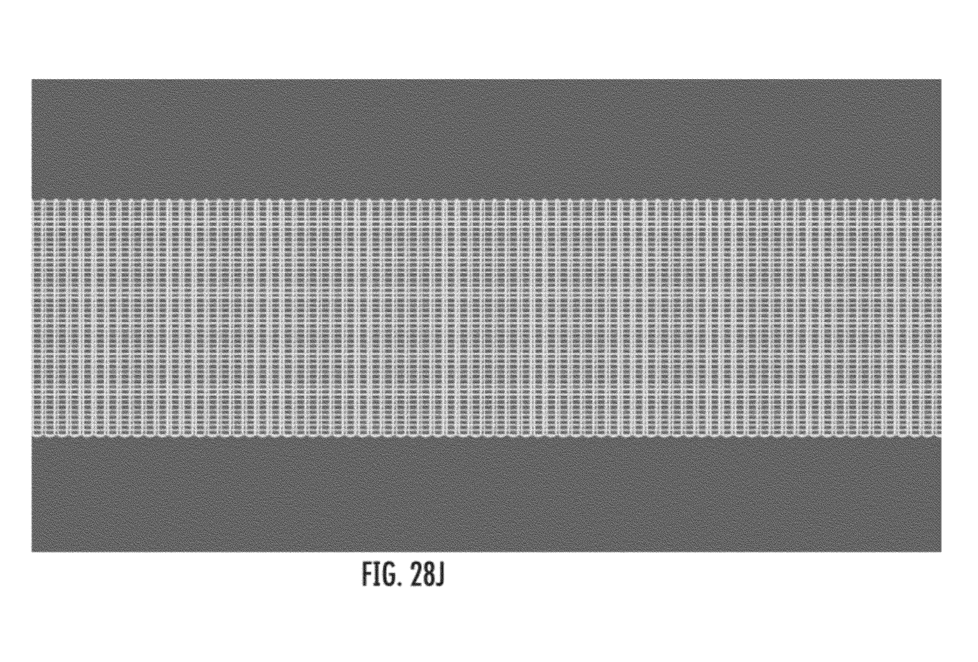

FIG. 28A is a photograph of a pattern layout for a silk-based mesh design suitable for use as a mesh in accordance with aspects of the present invention.

One example mesh in accordance with aspects of the present invention is preferably formed on a raschel knitting machine such as Comez DNB/EL-800-8B set up in 10 gg needle spacing by the use of three movements as shown in pattern layout in FIGS. 28B and C: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements as shown in FIGS. 28D and E and 1/1-1/3-3/3-3/1 for the other wale direction movement as shown in FIGS. 28H and I. The interlacing of the loops within the fabric allows for one yarn to become under more tension than the other under stress, locking it around the less tensioned yarn; keeping the fabric from unraveling when cut. The other movement in the course direction as shown in FIGS. 28F and 28G occurs in every few courses creating the porous design of the mesh. These yarns follow a repeat pattern of 9/9-9/9-7/7-9/9-7/7-9/9-7/7-9/9-7/7-9/9/-1/1-1/1-3/3-1/1-3/3-1/1-3/3-1/1-3/3-1/1 for the course direction movement. The pattern simulation layout of this pattern is rendered with ComezDraw 3 software in FIG. 28J considering a yarn design made with 2 ends of Td (denier count) 20/22 raw silk twisted together in the S direction to form a ply with 6 tpi (turns per inch) and further combining three of the resulting ply with 3 tpi. The same yarn design is used for the movements occurring in the wale and course directions. The stitch density or pick count for the mesh in FIG. 28J is 34 picks per centimeter considering the total picks count for the technical front face and the technical back face of the fabric, or 17 picks per cm considering only on the face of the fabric. The operating parameters are not limited to those described in FIG. 28B-I, but just the optimum values for the specific yarn design used for the pattern simulation layout of FIG. 28J.

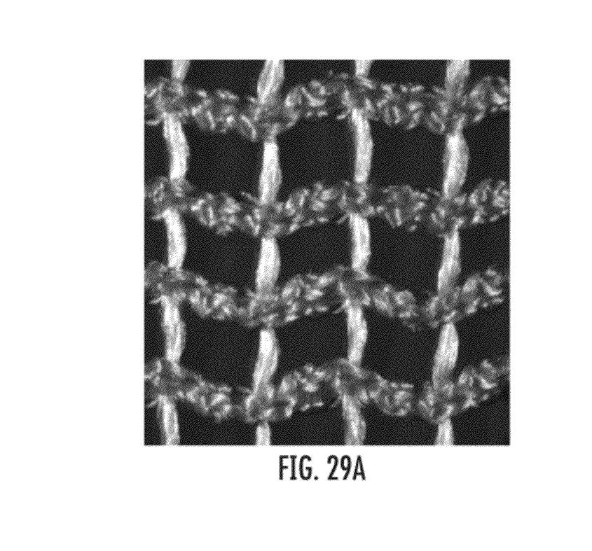

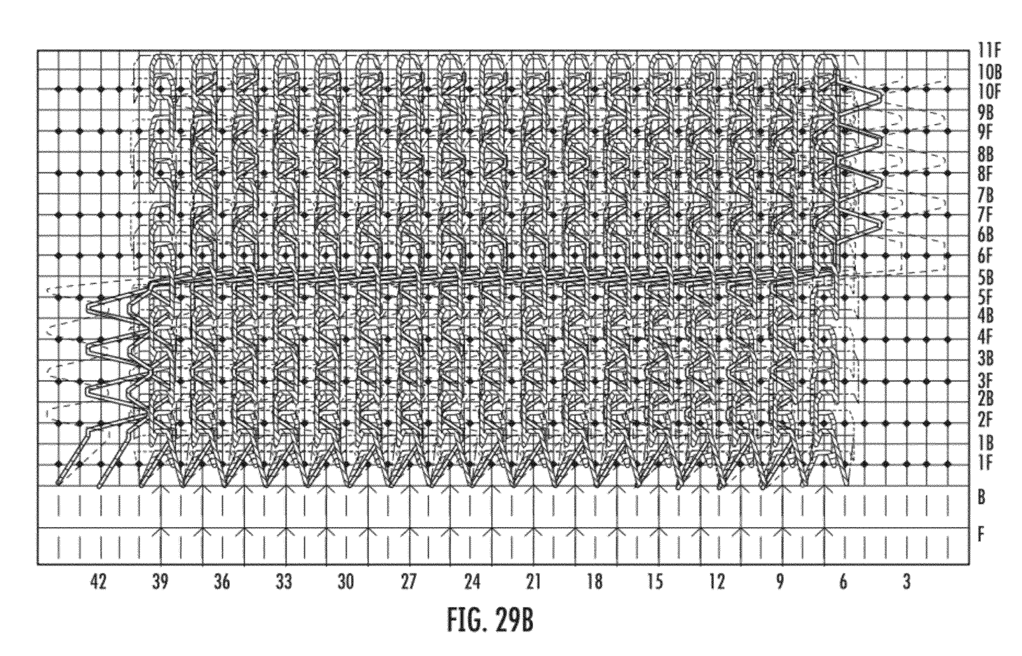

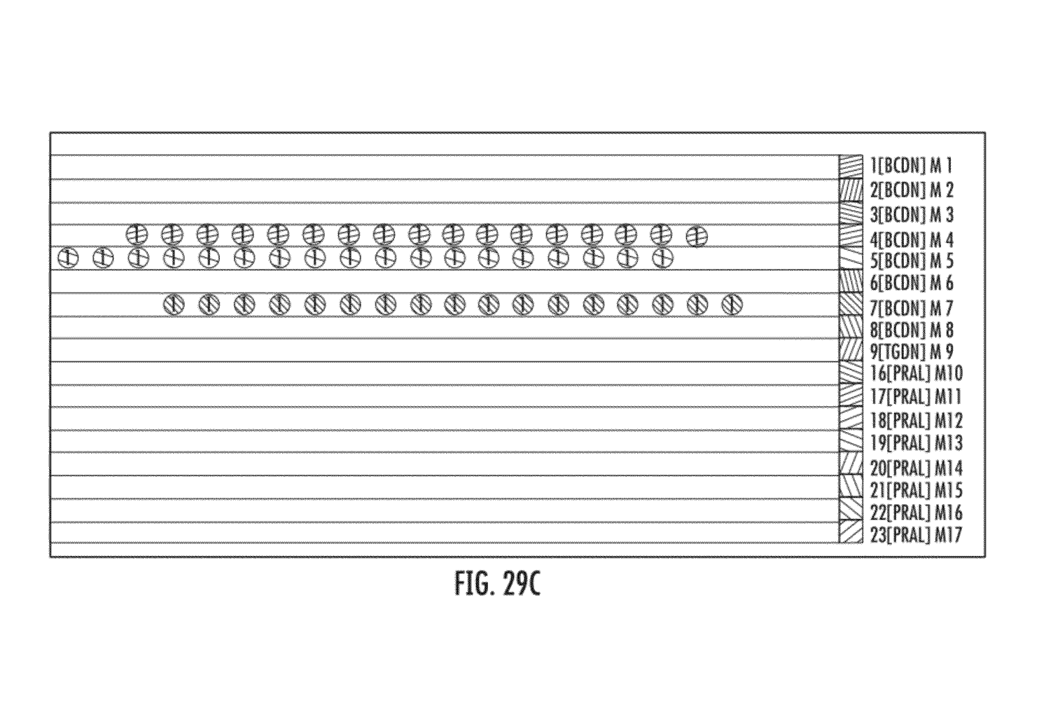

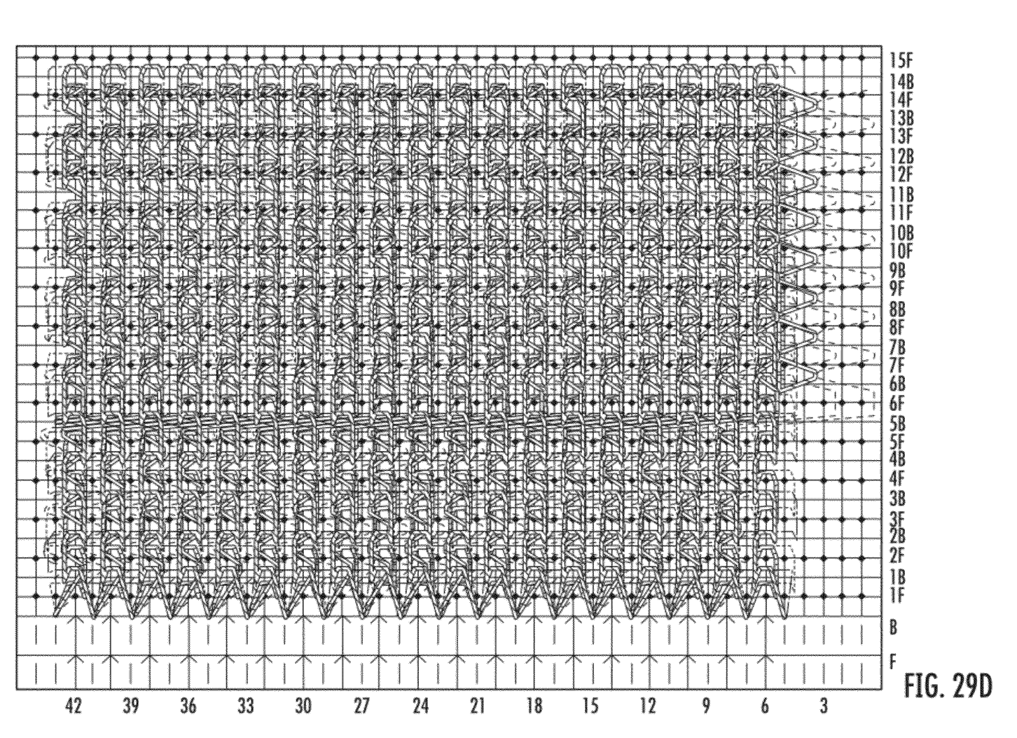

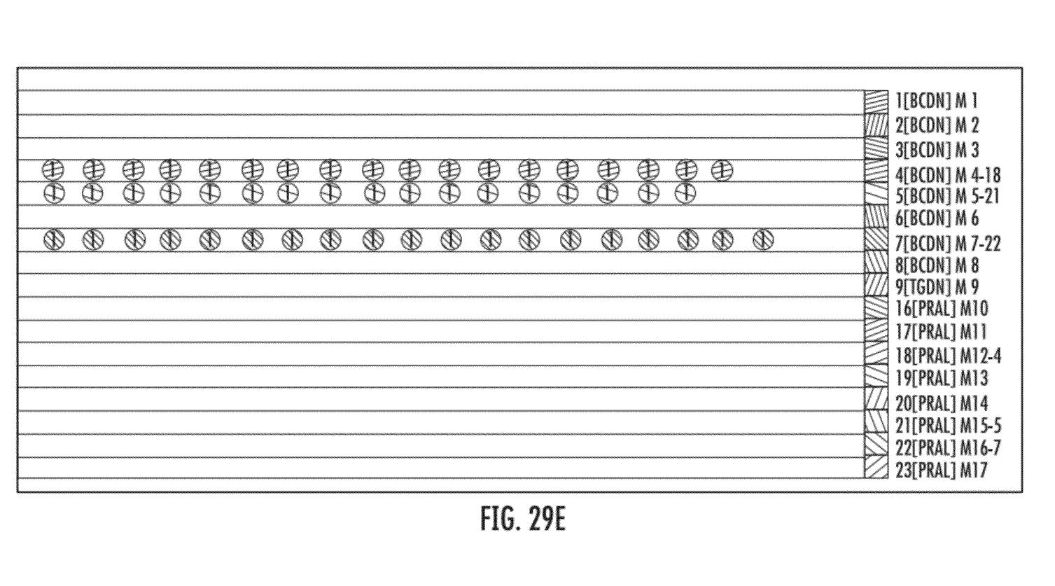

FIG. 29A illustrates a photograph of a pattern layout for a silk-based mesh in accordance with aspects of the present invention.

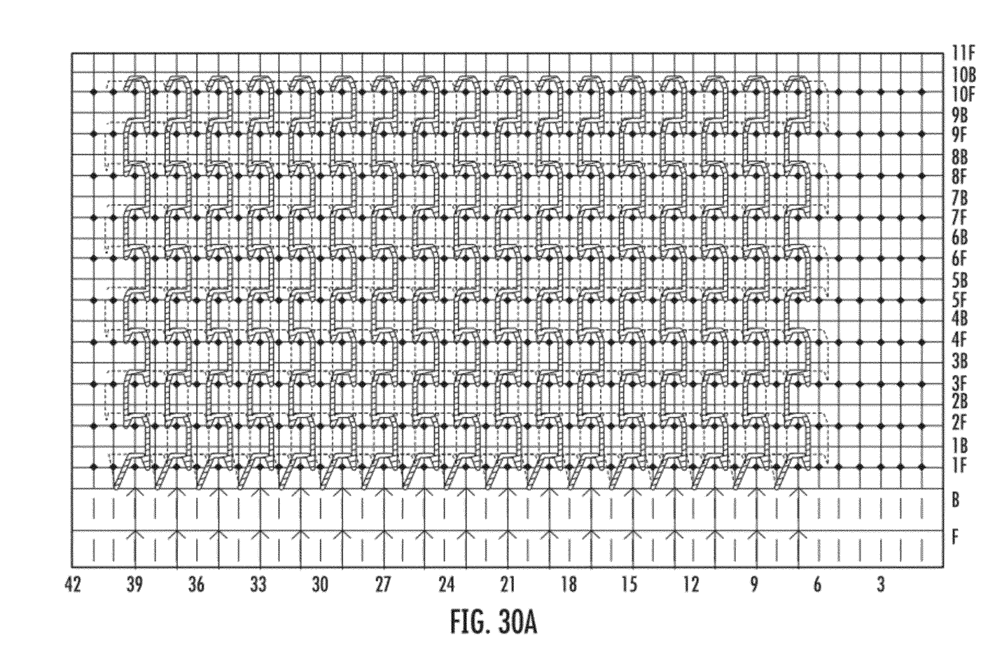

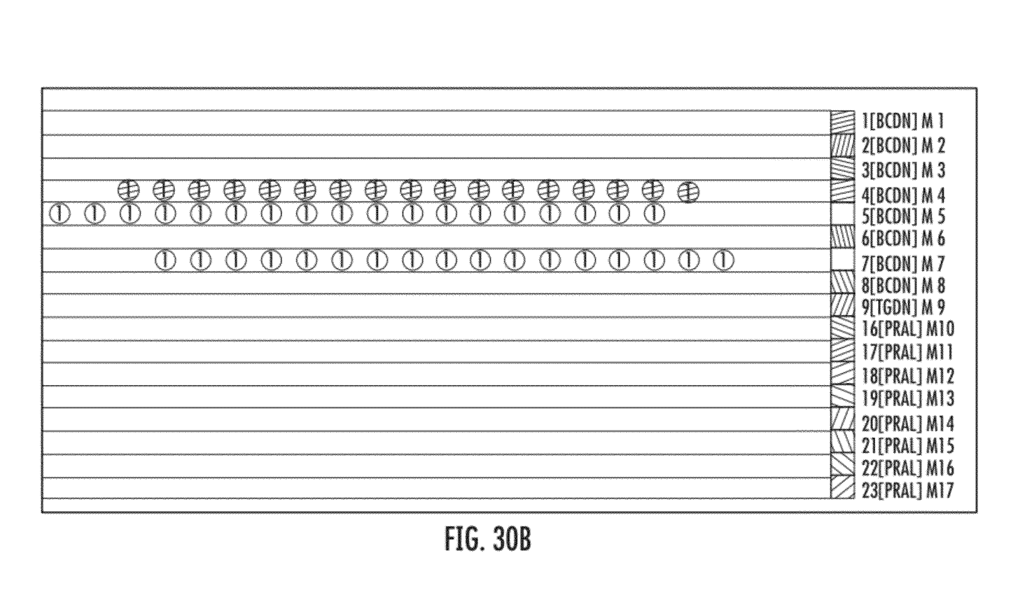

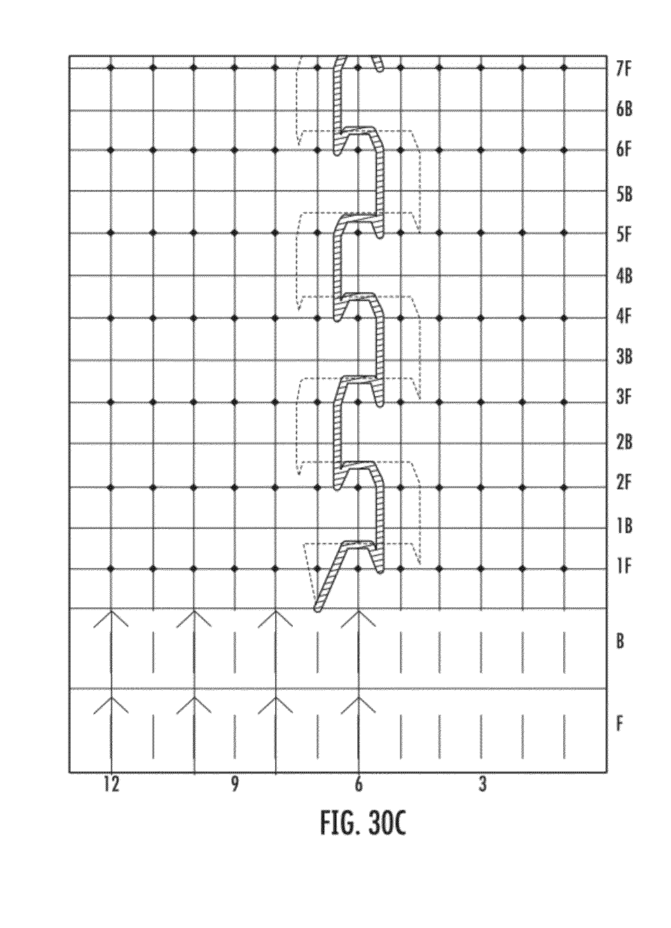

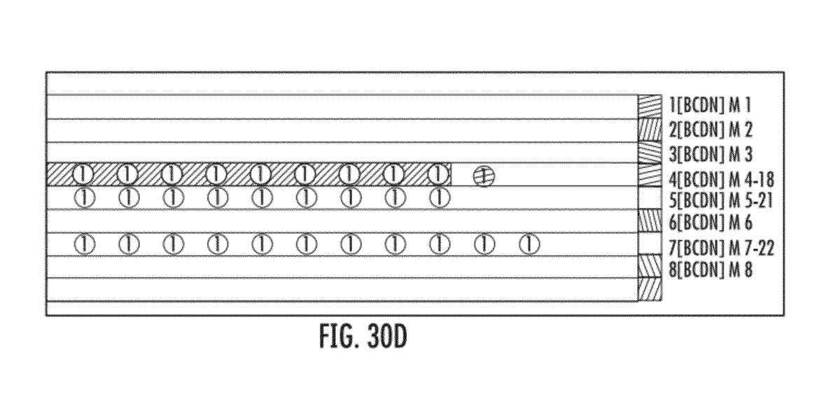

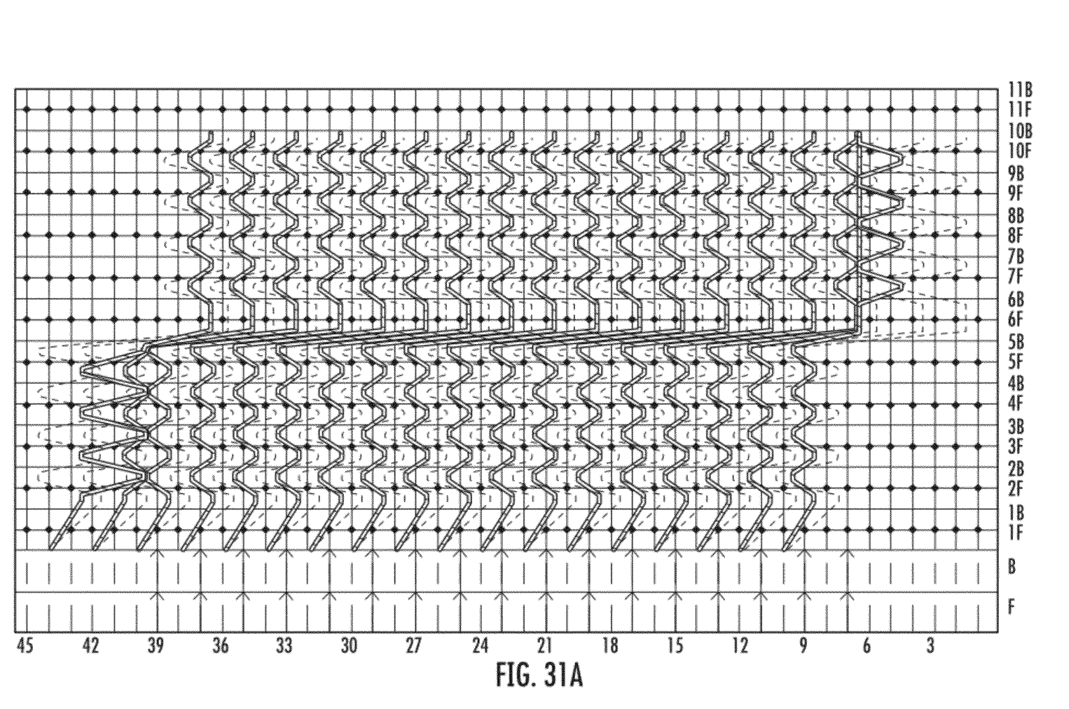

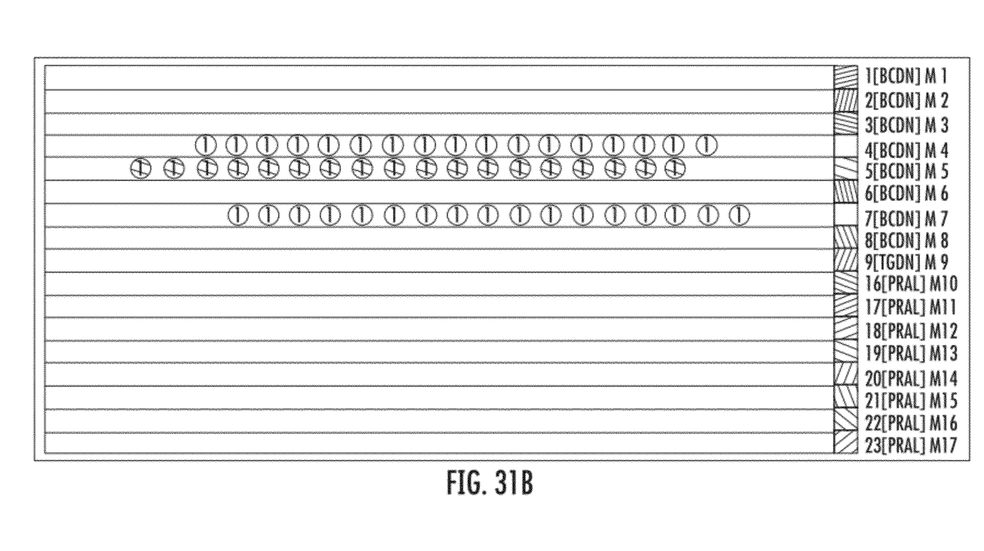

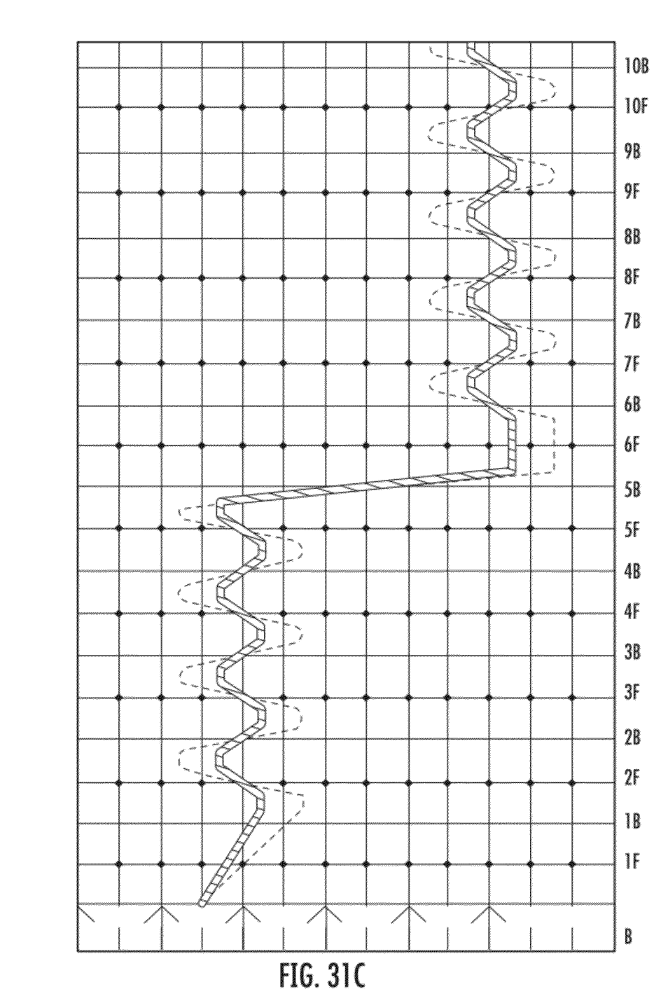

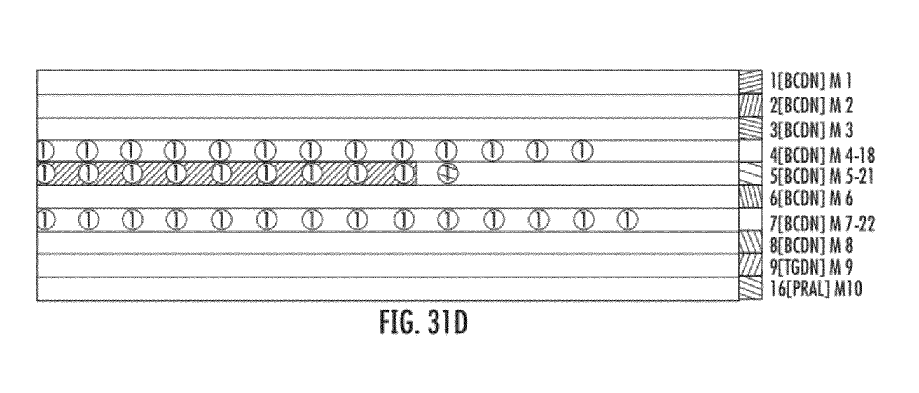

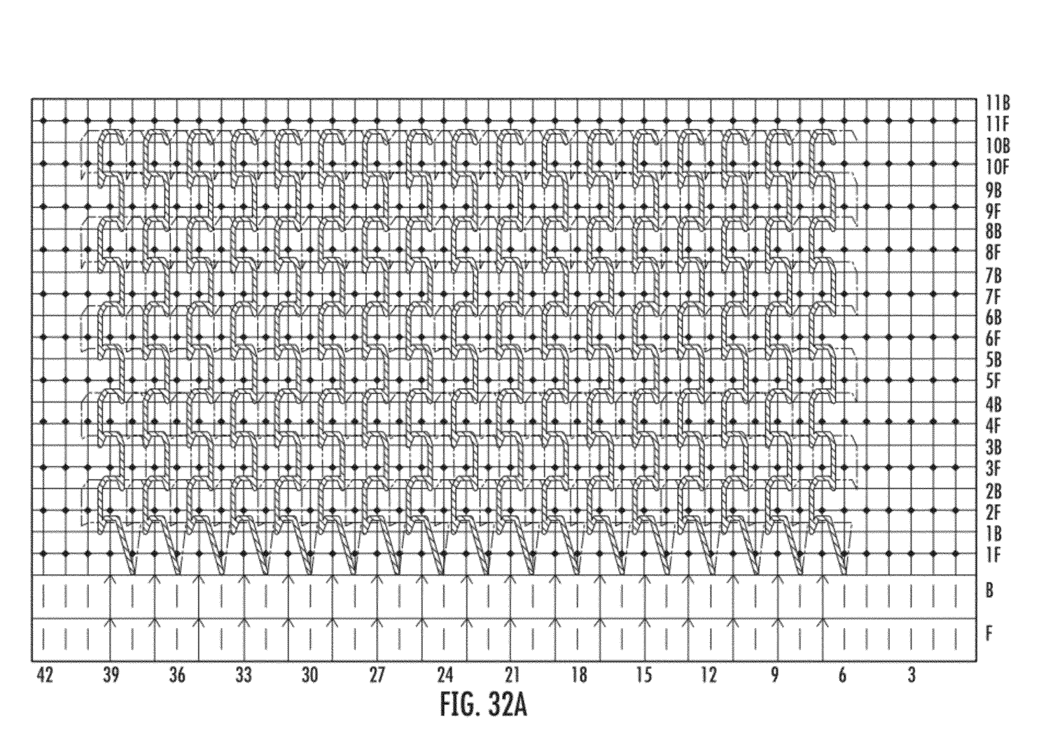

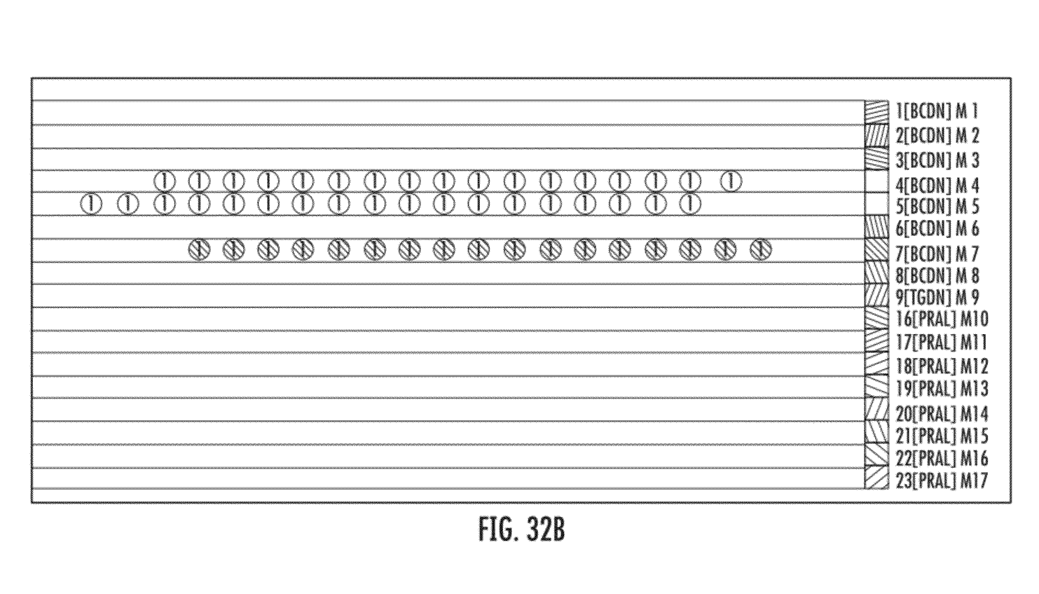

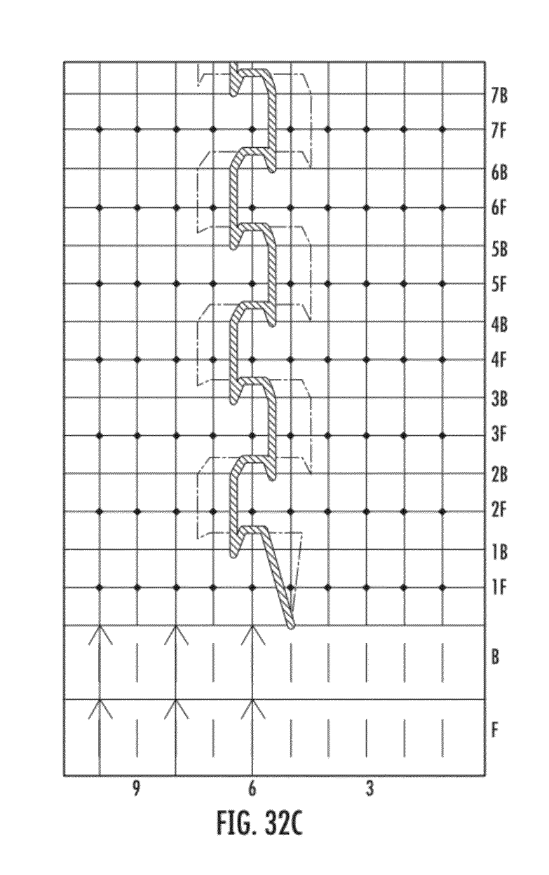

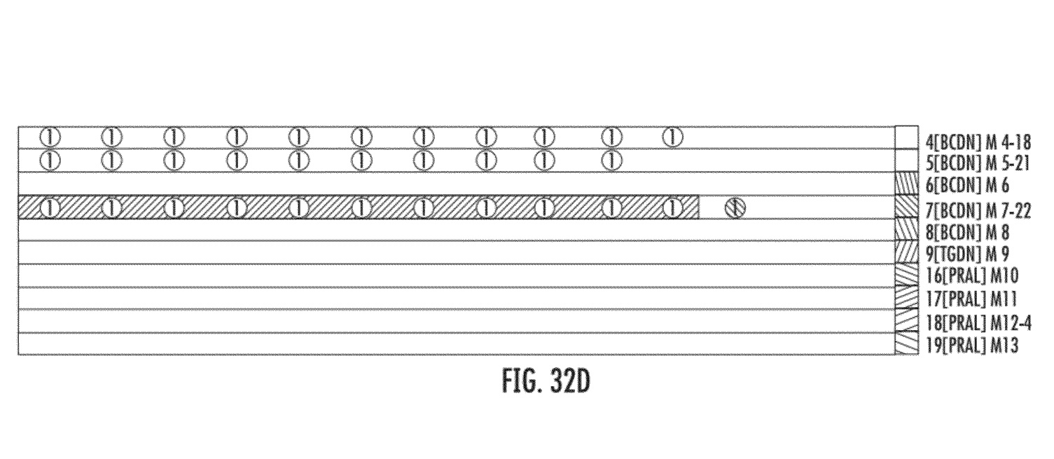

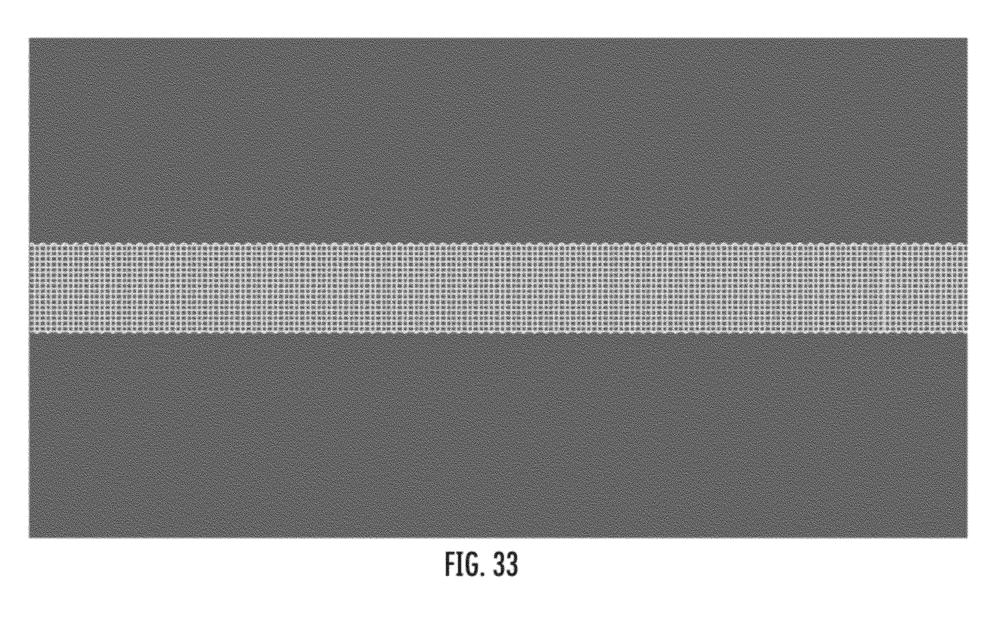

One variation of the mesh in accordance with aspects of the present invention is preferably formed on a raschel knitting machine such as Comez DNB/EL-800-8B set up in 10 gg needle spacing by the use of three movements as shown in pattern layout in FIGS. 29B-E: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements (see ground bar #4) as shown in FIGS. 30A and B and FIGS. 30C and D and 1/1-1/3-3/3-3/1 for the other wale direction movement (see ground bar #7) as shown in FIGS. 32A and B, FIGS. 32C and D. The interlacing of the loops within the fabric allows for one yarn to become under more tension than the other under stress, locking it around the less tensioned yarn; keeping the fabric from unraveling when cut. The other movement in the course direction as shown in FIG. 31 occurs in every few courses creating the porous design of the mesh. These yarns follow a repeat pattern of 9/9-9/9-7/7-9/9-7/7-9/9-7/7-9/9-7/7-9/9/-1/1-1/1-3/3-1/1-3/3-1/1-3/3-1/1-3/3-1/1 (see ground bar #5) for the course direction movement as shown in FIGS. 31A and B and FIGS. 31C and D. The pattern simulation layout of this pattern is rendered with ComezDraw 3 software in FIG. 33 considering a yarn design made with 2 ends of Td (denier count) 20/22 raw silk twisted together in the S direction to form a ply with 6 tpi (turns per inch) and further combining three of the resulting ply with 3 tpi. The same yarn design is used for the movements occurring in the wale and course directions. The stitch density or pick count for the mesh in FIG. 33 is 40 picks per centimeter considering the total picks count for the technical front face and the technical back face of the fabric, or 20 picks per cm considering only on the face of the fabric. The operating parameters are not limited to those described in FIGS. 29B-E, but are merely the optimum values for the specific yarn design used for the pattern simulation layout of FIG. 33.

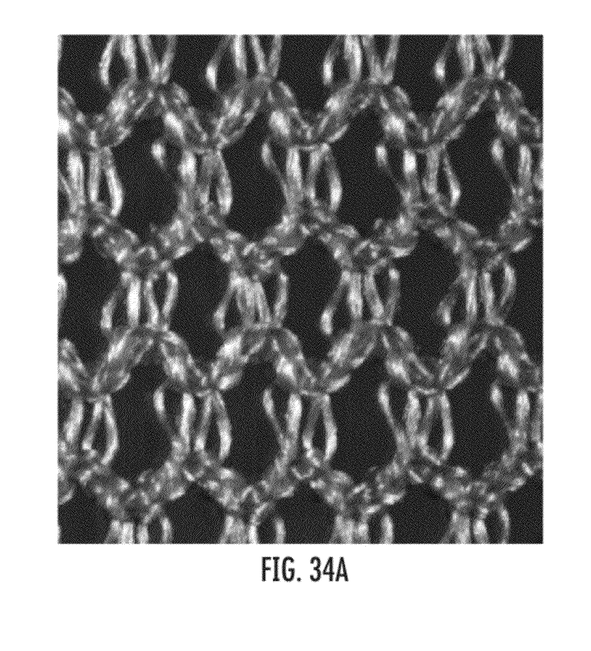

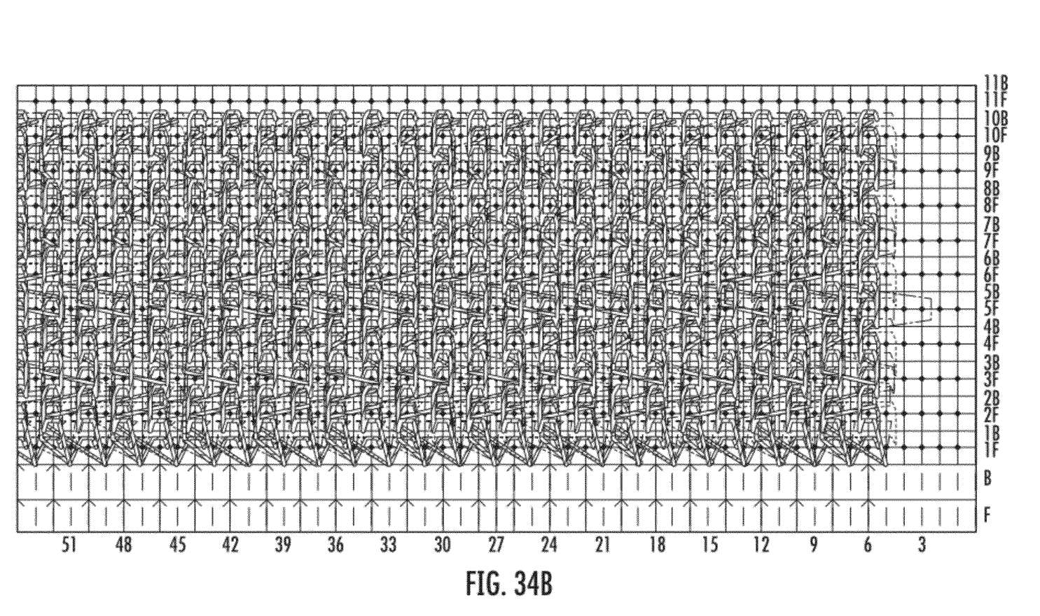

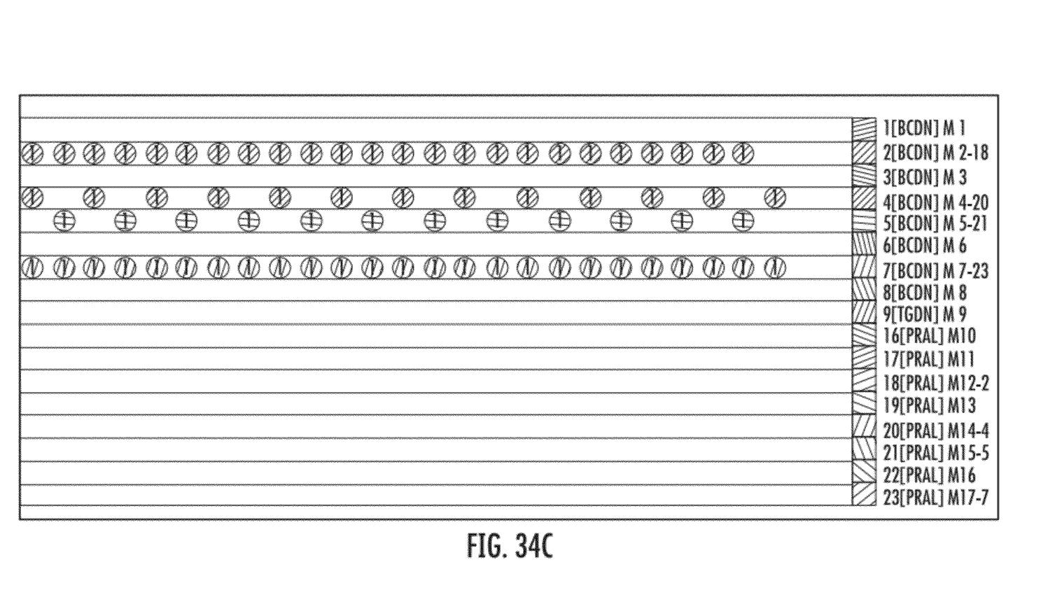

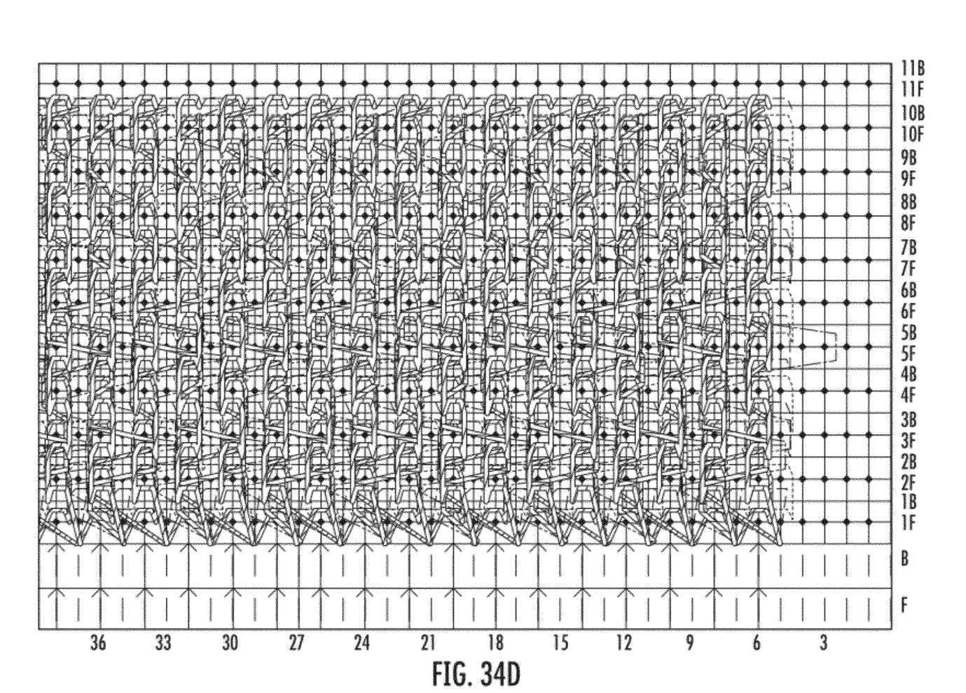

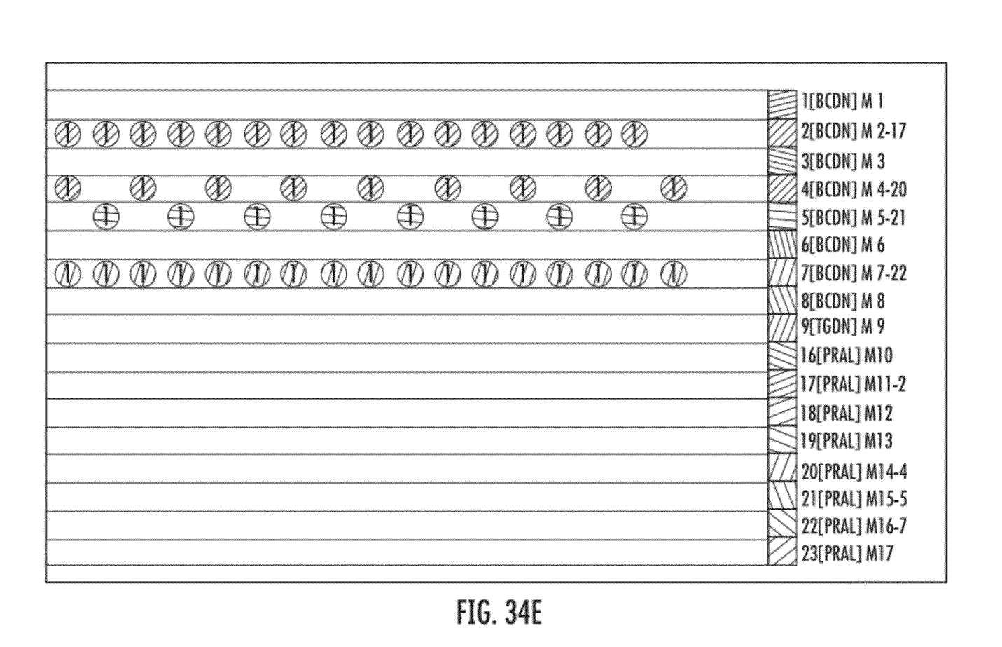

FIG. 34A is a photograph of a pattern layout for a silk-based mesh in accordance with aspects of the present invention.

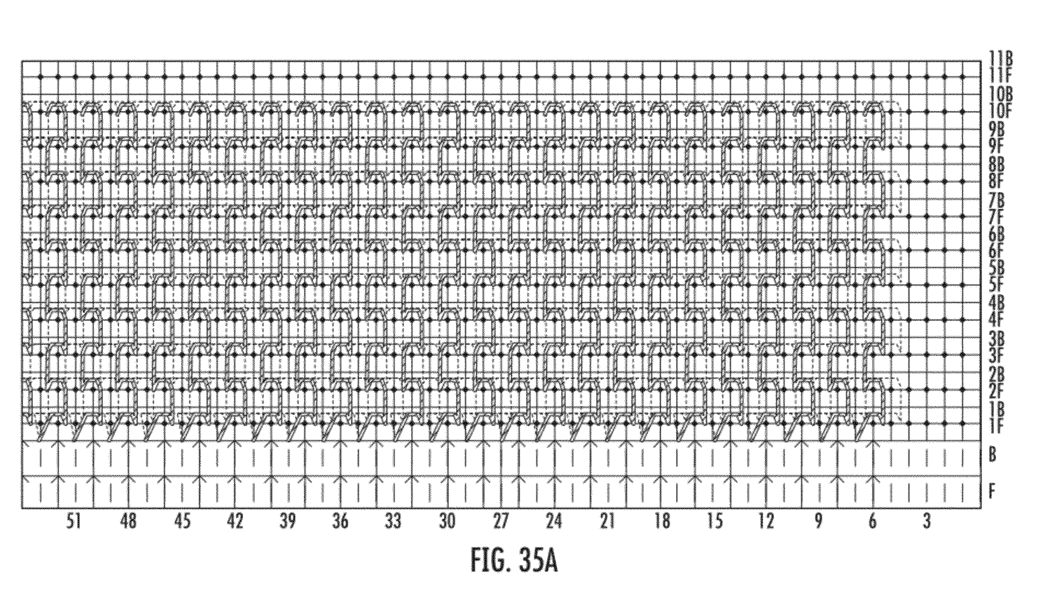

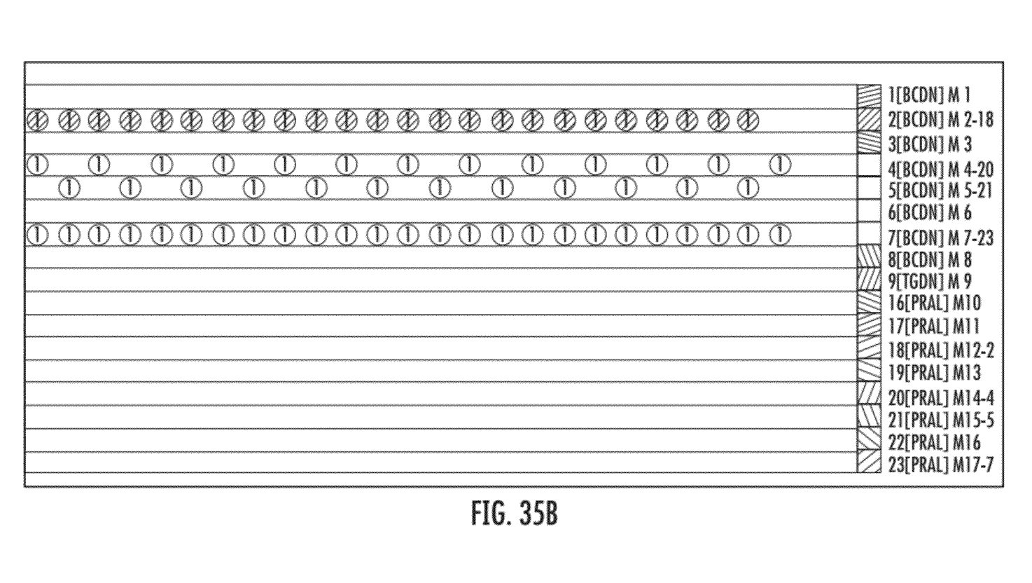

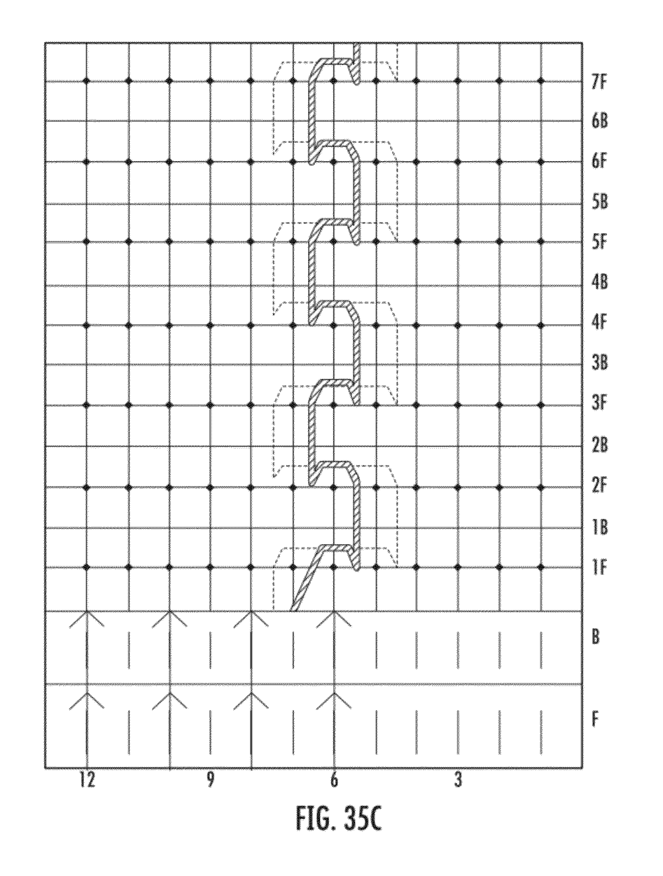

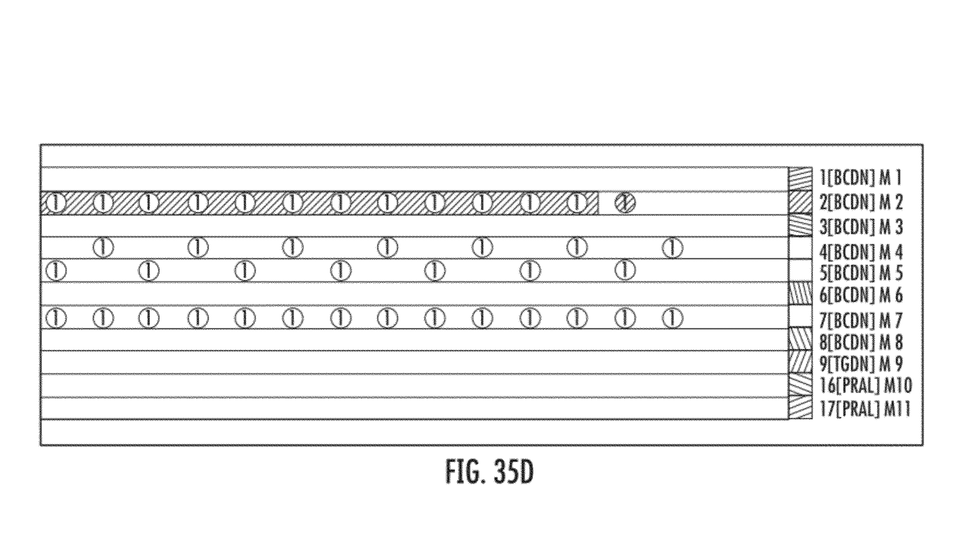

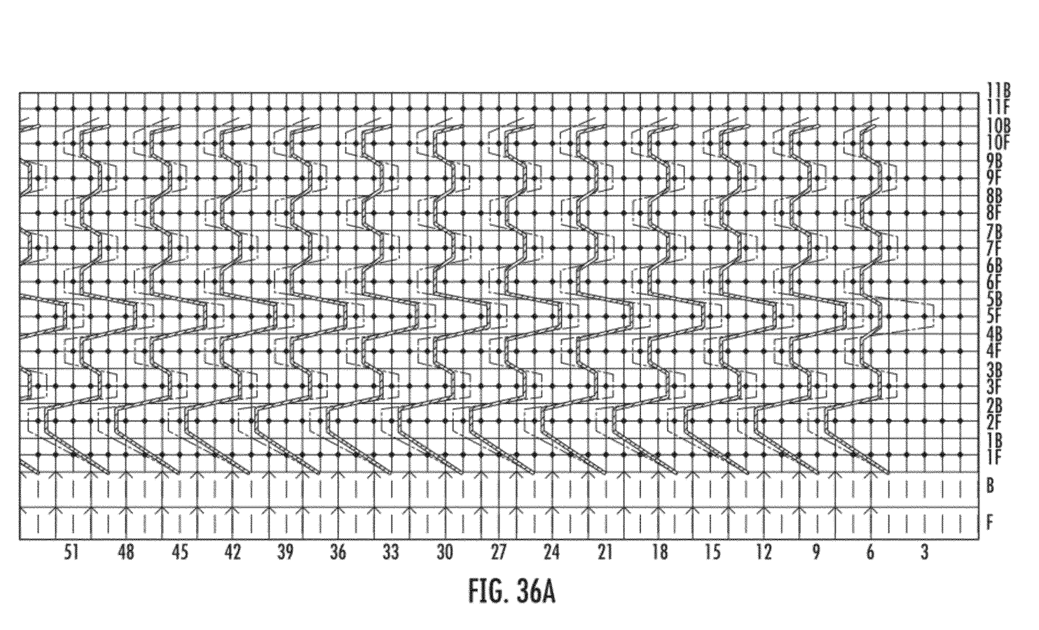

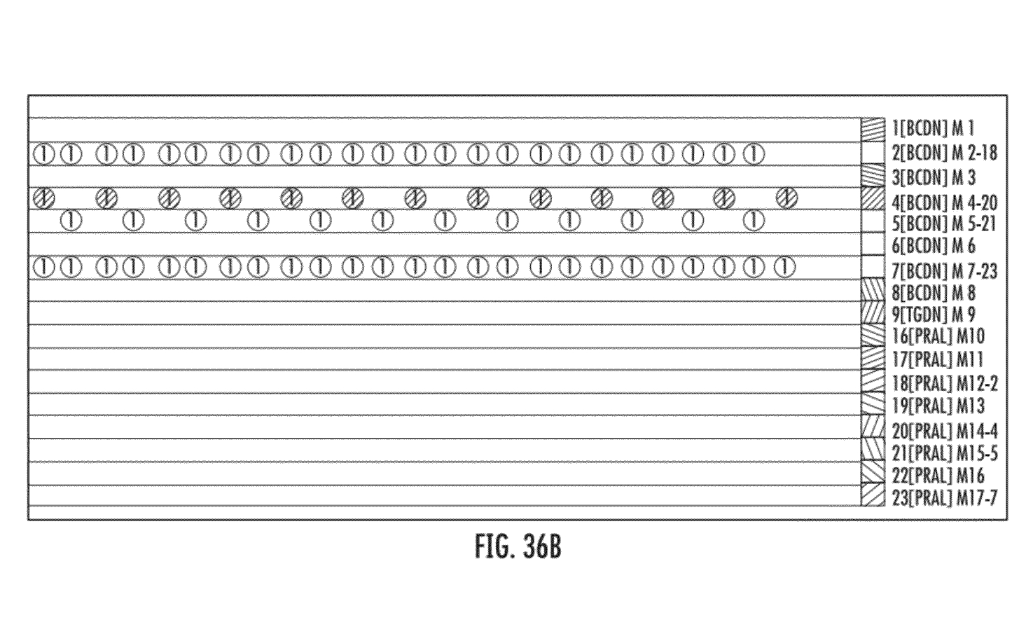

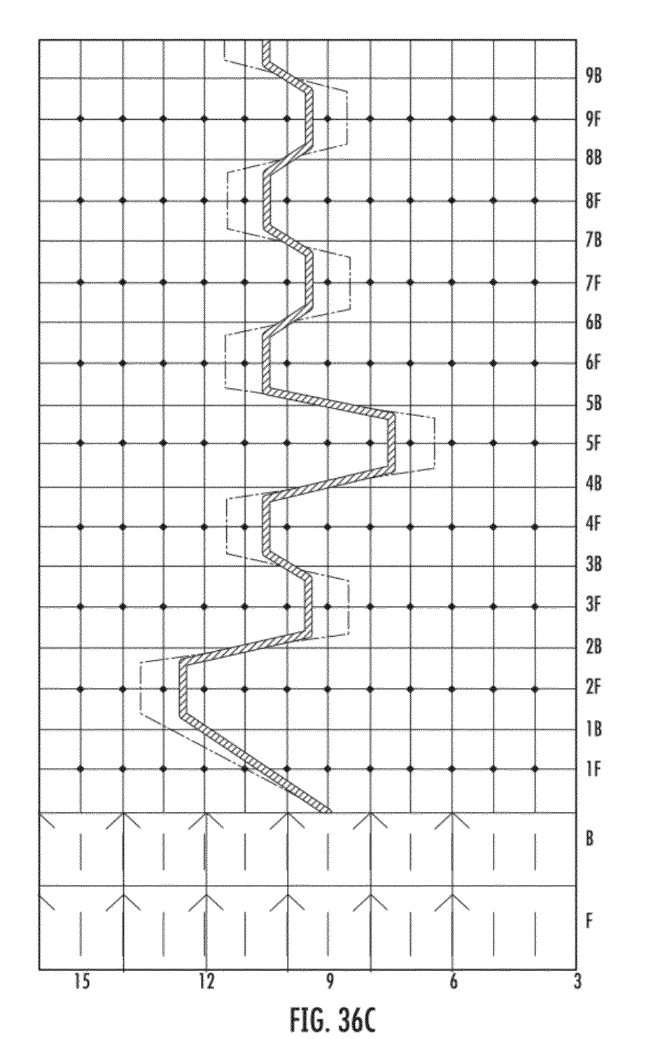

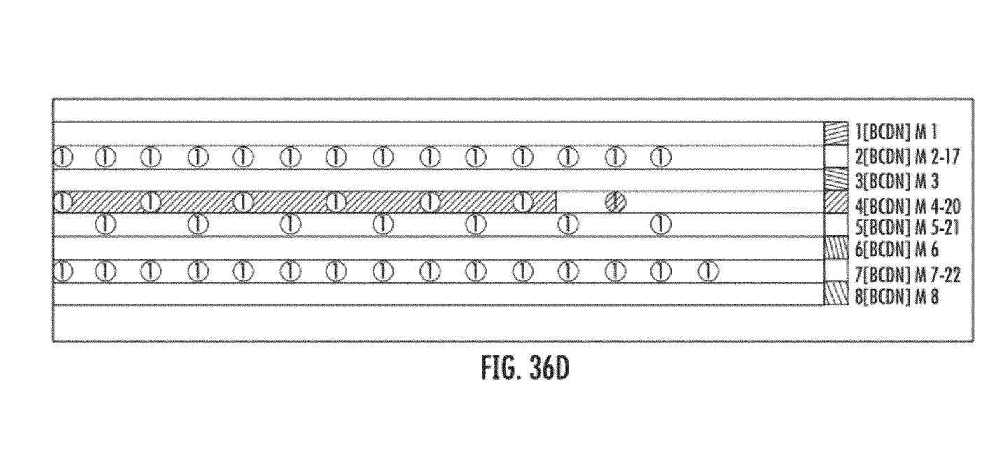

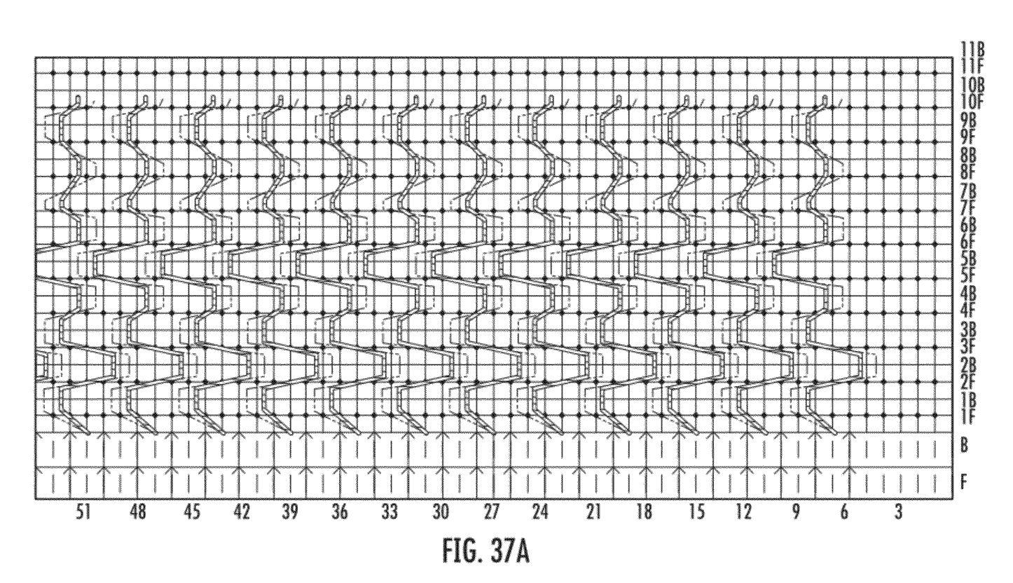

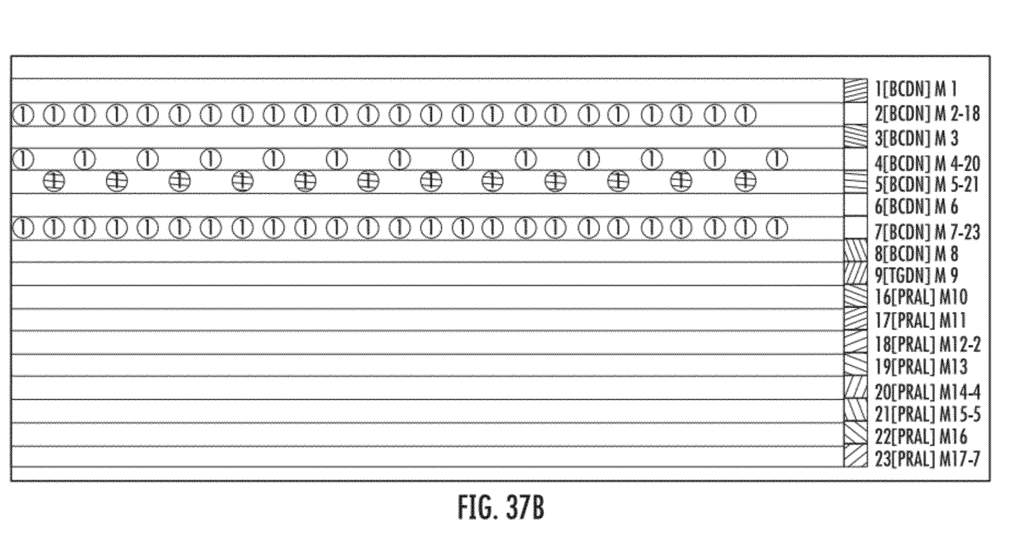

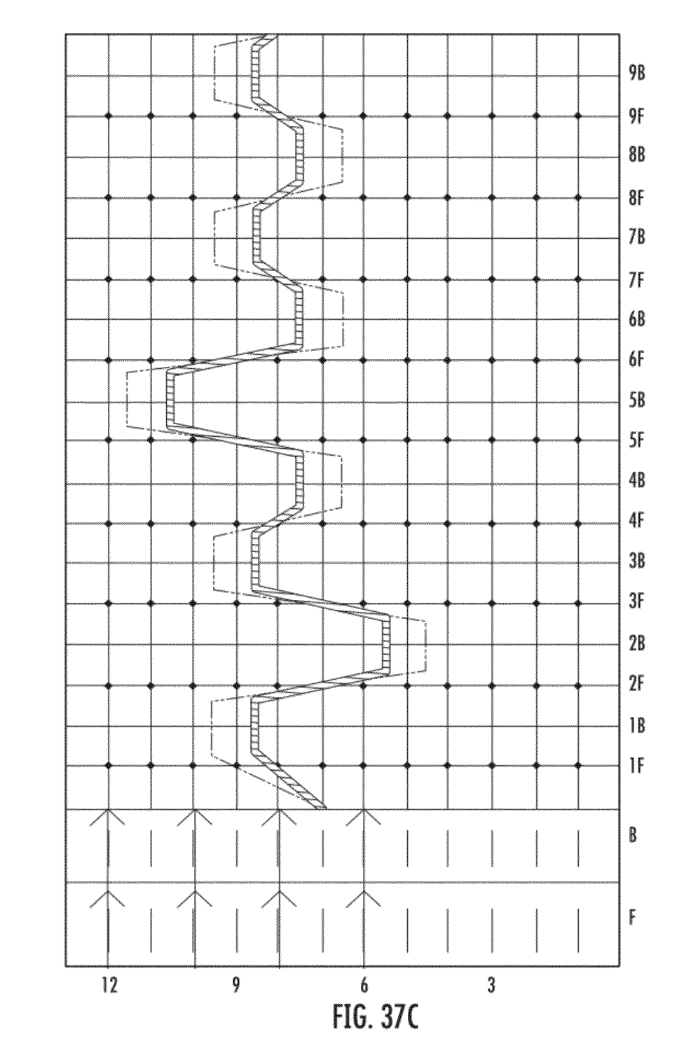

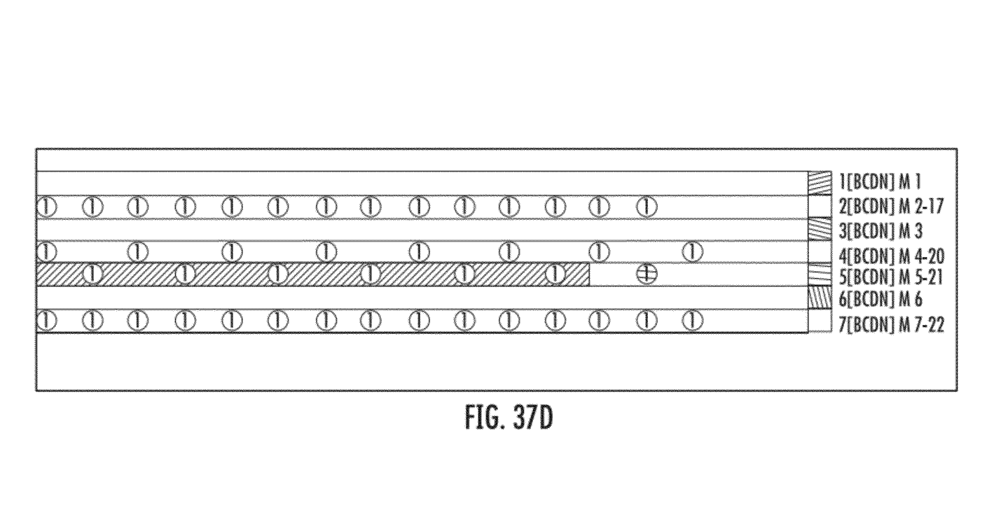

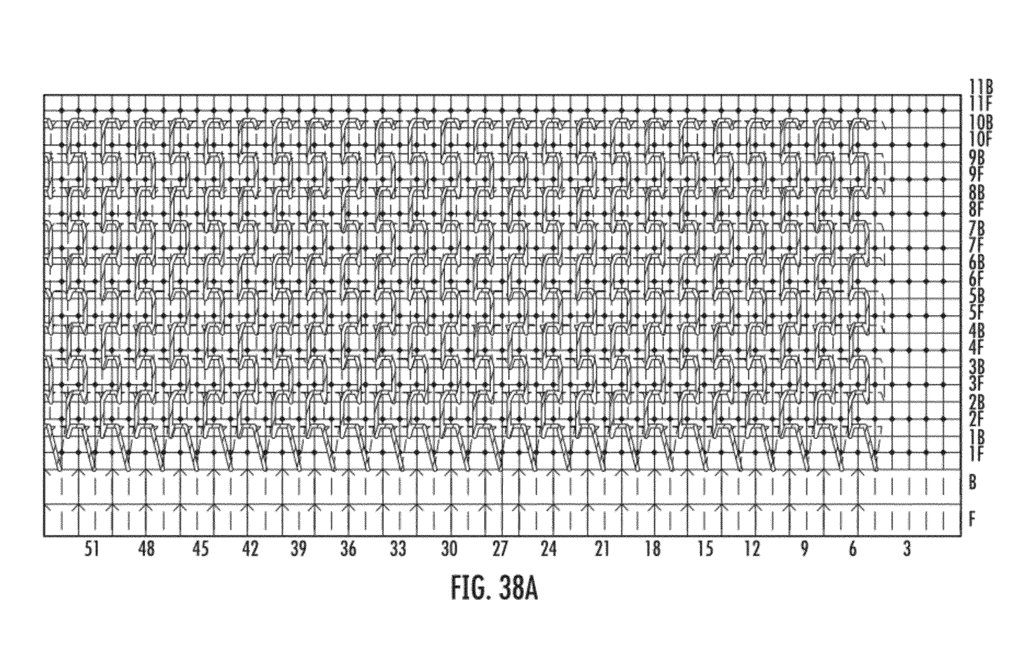

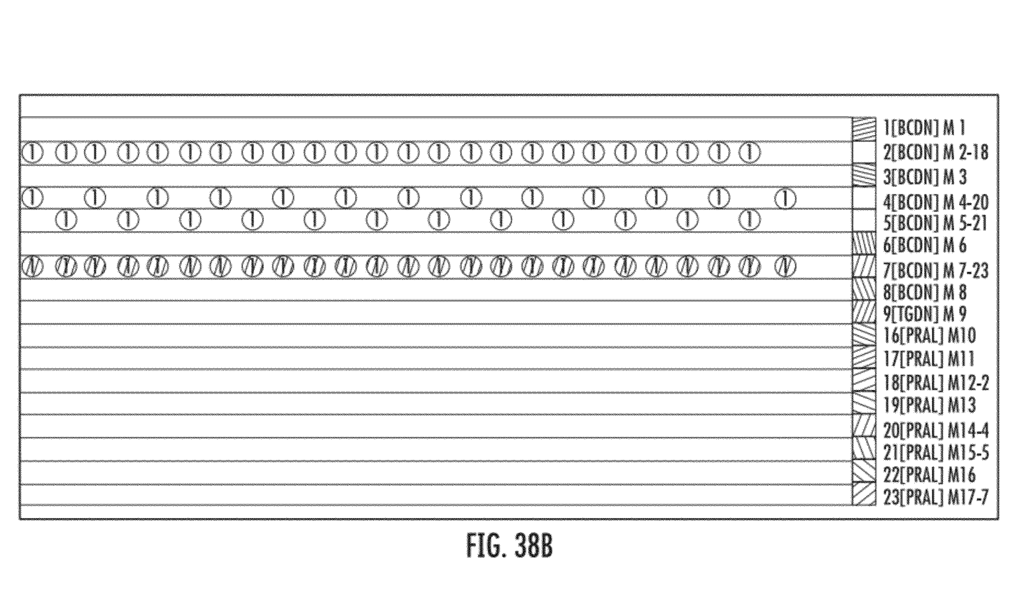

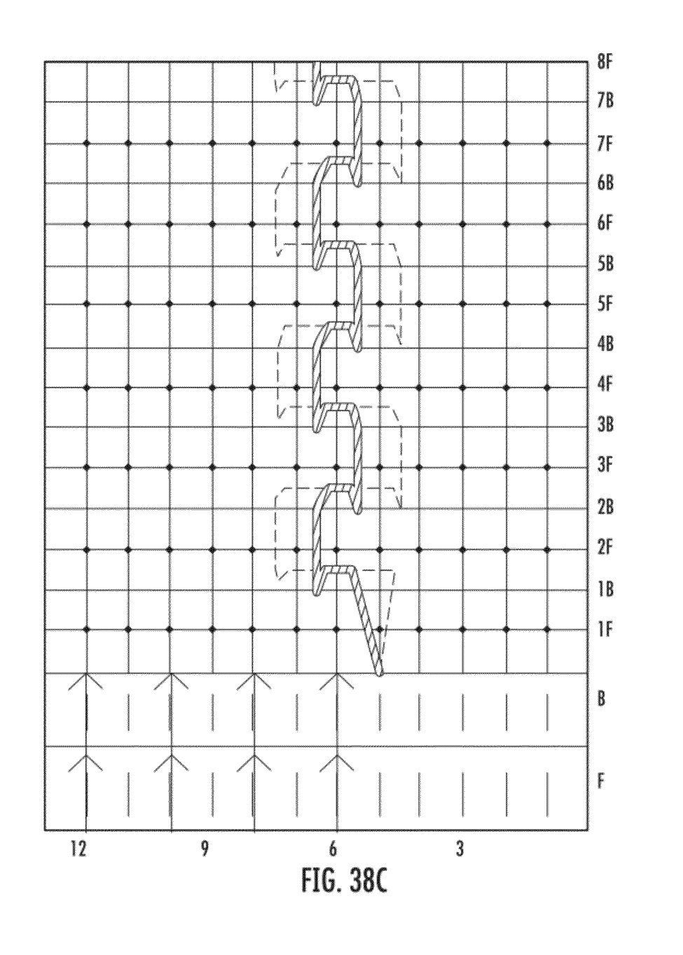

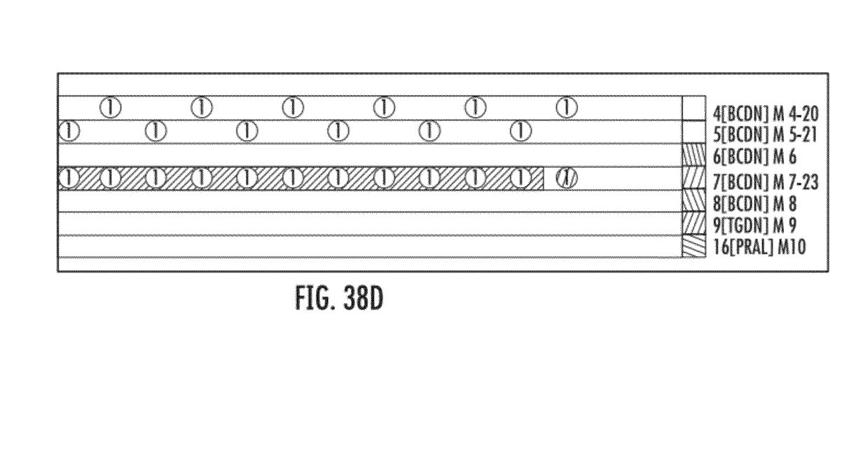

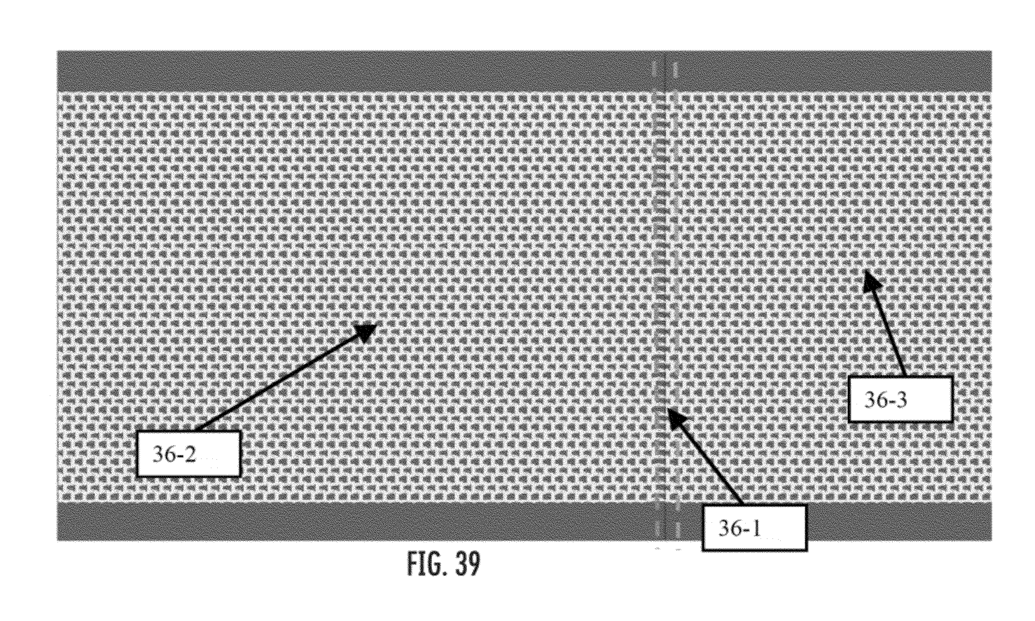

Another variation of the mesh in accordance with aspects of the present invention is preferably created on a raschel knitting machine such as Comez DNB/EL-800-8B set up in 10 gg needle spacing by the use of four movements as shown in pattern layout in FIGS. 34B and C and FIGS. 34D and E: two movements in the wale direction, the vertical direction within the fabric, and two movements in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements as shown in FIGS. 35A-D and 1/1-1/3-3/3-3/1 for the other wale direction movement as shown in FIGS. 38A-D. The interlacing of the loops within the fabric allows for one yarn to be under more tension than the other under stress, locking it around the less tensioned yarn; keeping the fabric from unraveling when cut. One of the other two movements in the course direction as shown in FIGS. 36A-D occurs in every few courses creating the porous design of the mesh. These yarns follow a repeat pattern of 3/3-3/3-7/7-7/7-3/3-3/3-5/5-5/5-1/1-1/1-5/5-5/5-3/3-3/3-5/5-5/5-3/3-3/3-5/5-5/5 for the course direction movement. The other movements in the course direction as shown in FIGS. 37A-D occur in every few courses creating the openings in the mesh. These yarns follow a repeat pattern of 3/3-3/3-5/5-5/5-1/1-1/1-5/5-5/5-3/3-3/3-7/7-7/7-3/3-3/3-5/5-5/5-3/3-3/3-5/5-5/5-3/3 for the course direction movement. The pattern simulation layout of this pattern is rendered with ComezDraw 3 software in FIG. 39 considering a yarn design made with 2 ends of Td 20/22 raw silk twisted together in the S direction to form a ply with 6 tpi and further combining three of the resulting ply with 3 tpi. The same yarn design is used for the movements occurring in the wale and course directions. The stitch density or pick count for the surgical mesh design in FIG. 39 is 39 picks per centimeter considering the total picks count for the technical front face and the technical back face of the fabric, or 19.5 picks per cm considering only one face of the fabric. The operating parameters are not limited to those described in FIGS. 34B-E, but just the optimum values for the specific yarn design used for the pattern simulation layout of FIG. 39.

Furthermore, FIG. 39 demonstrates a process improvement for the manufacturing process of the mesh with the pattern layout in FIGS. 34B-E. The improvement consists of a separation area, 36-1, between two individual meshes, 36-2 and 36-3. The advantage of the separation area is to provide guidance for the correct length that the mesh needs to measure and to provide guidance for the tools necessary for separating two individual surgical meshes. For example in order to achieve the mesh length of 5 cm±0.4 cm, the pattern in FIGS. 34B-E requires repeating from pattern line 1 to pattern line 16 for 112 times followed by a repeat of 2 times from pattern line 17 to pattern line 20.

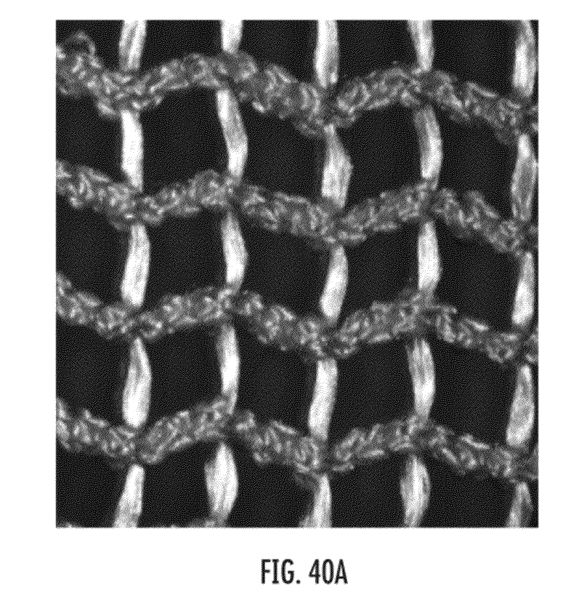

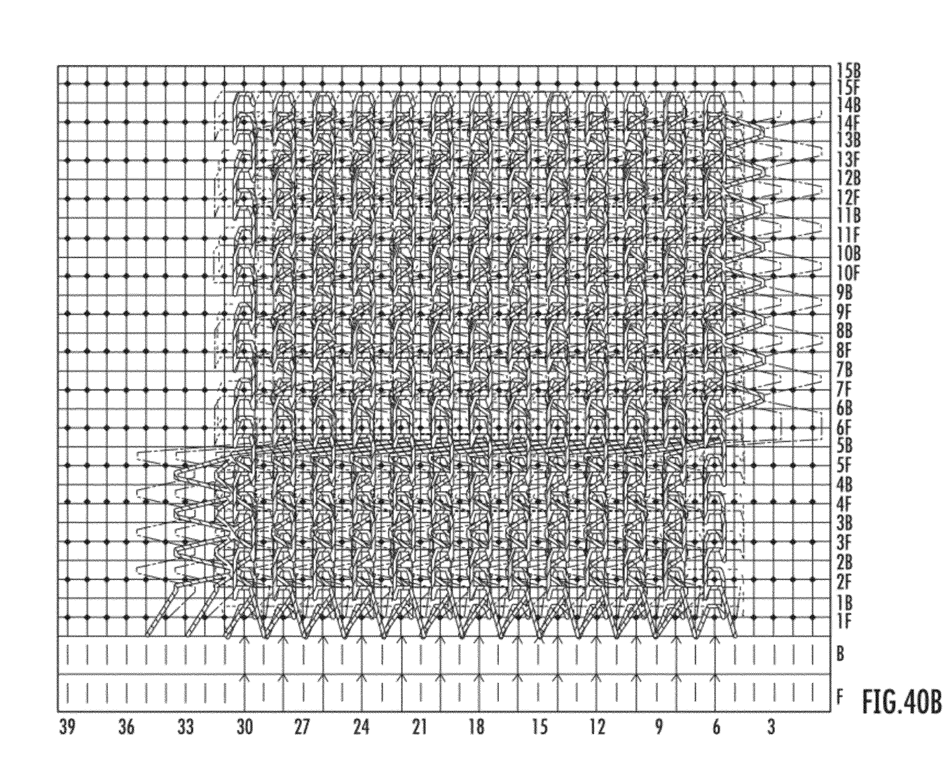

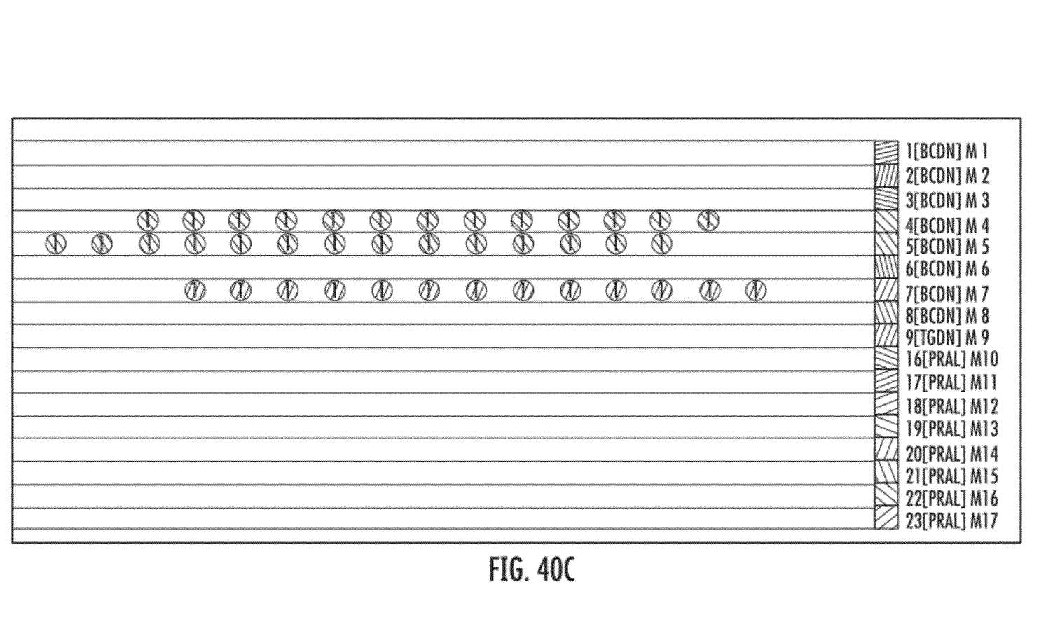

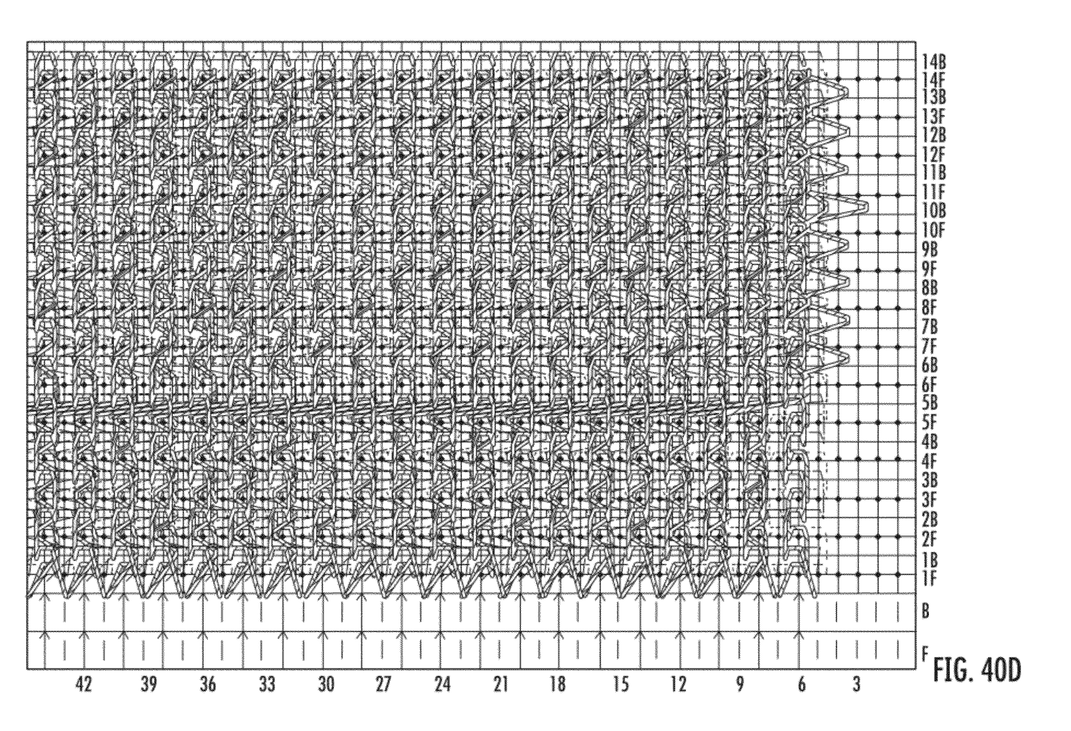

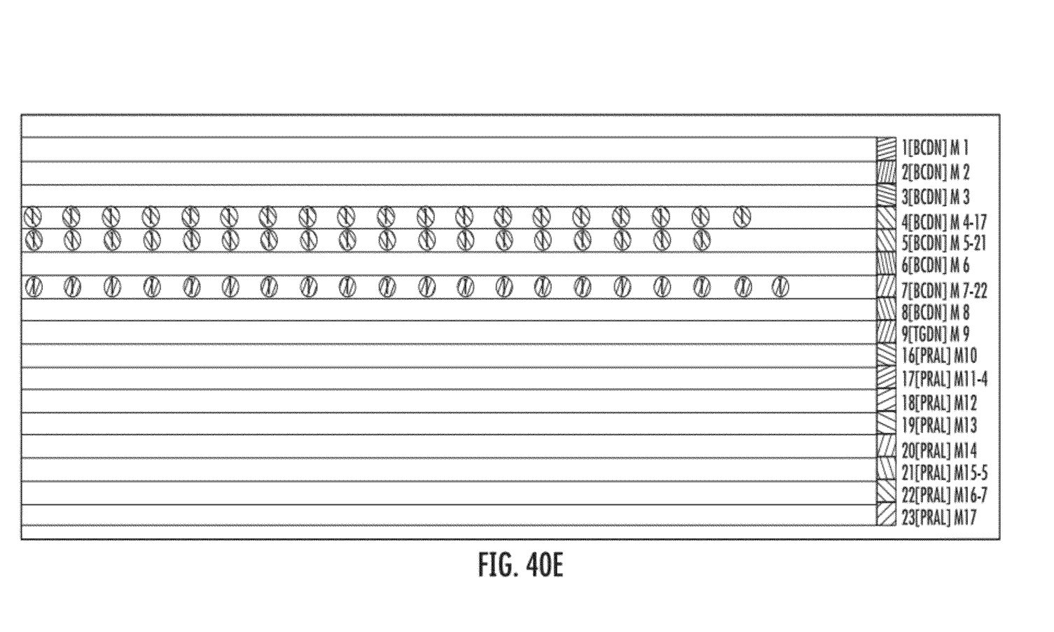

FIG. 40A is a photograph of a pattern layout for a silk-based mesh in accordance with aspects of the present invention.

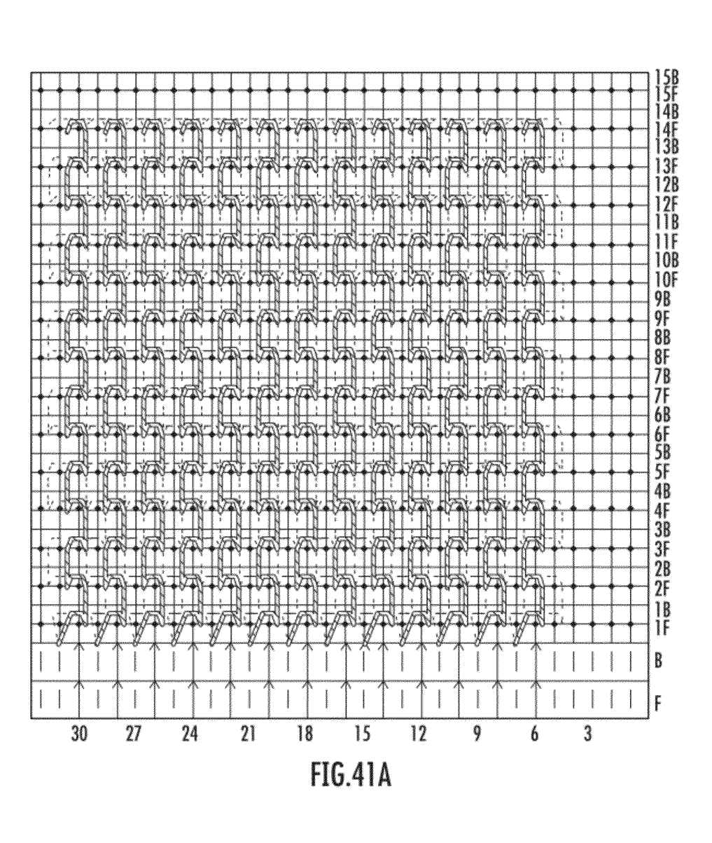

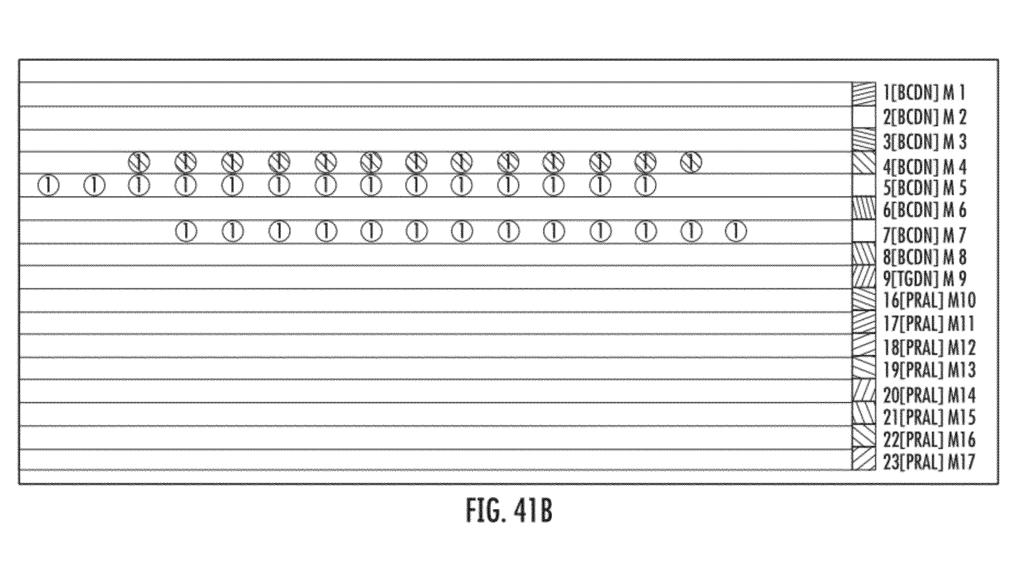

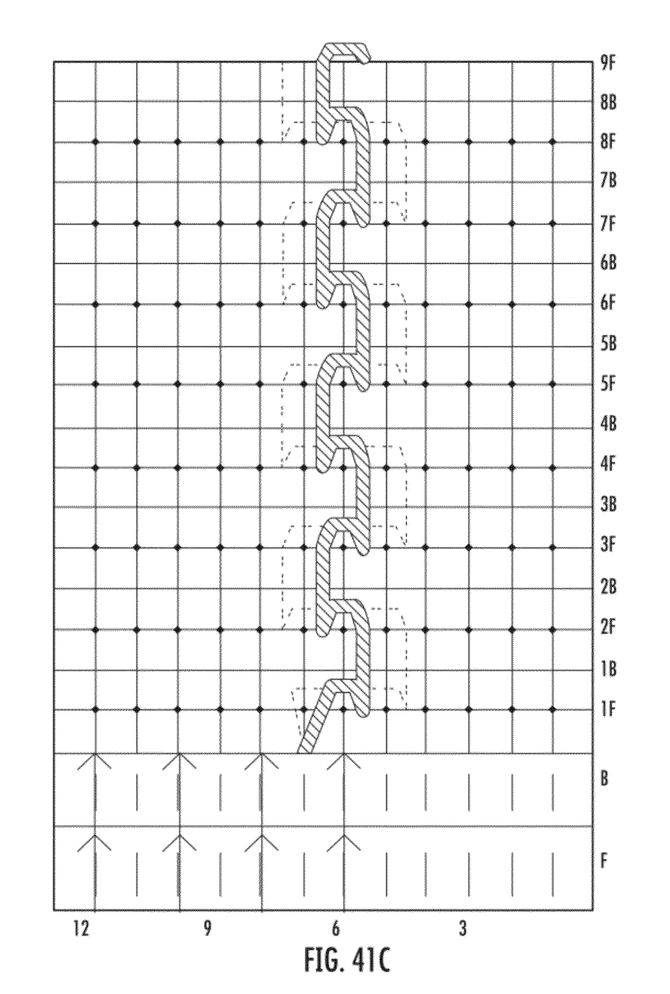

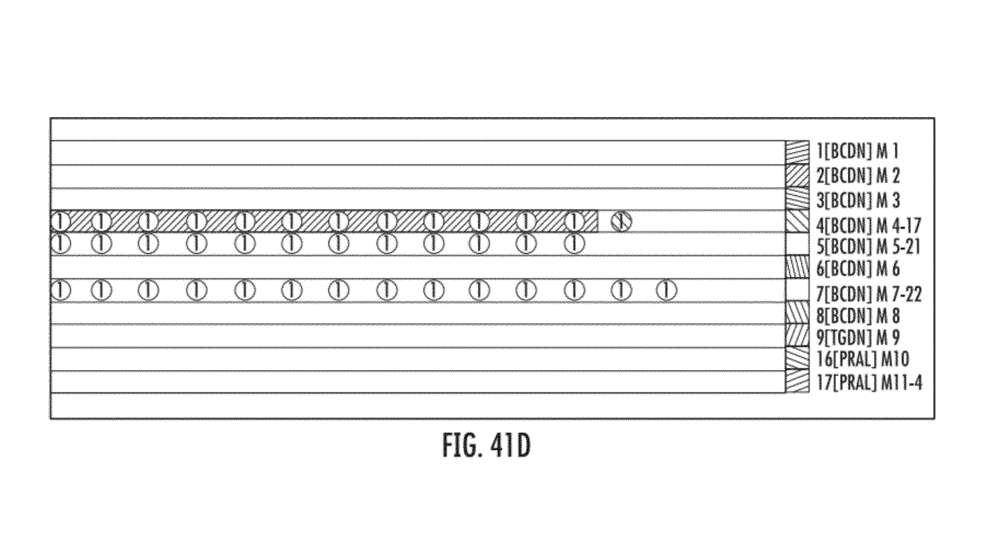

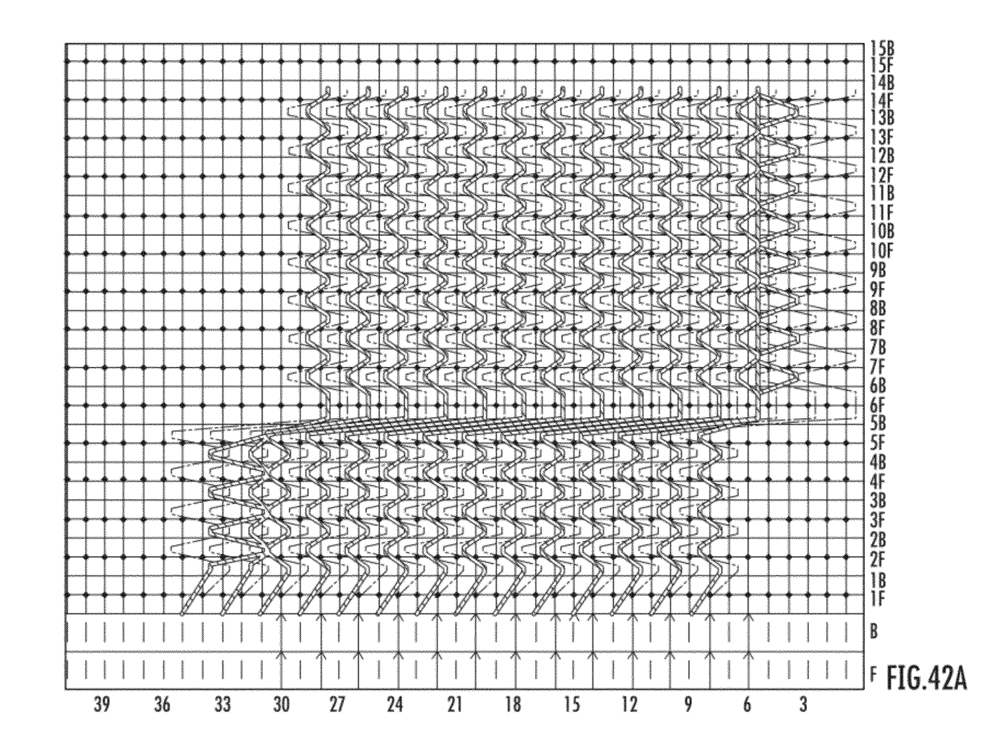

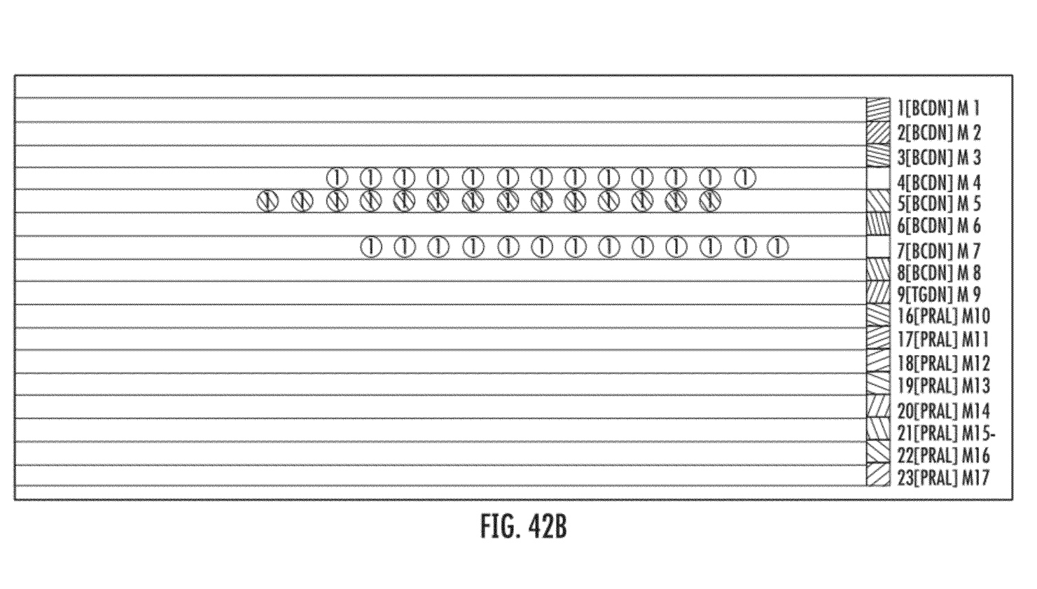

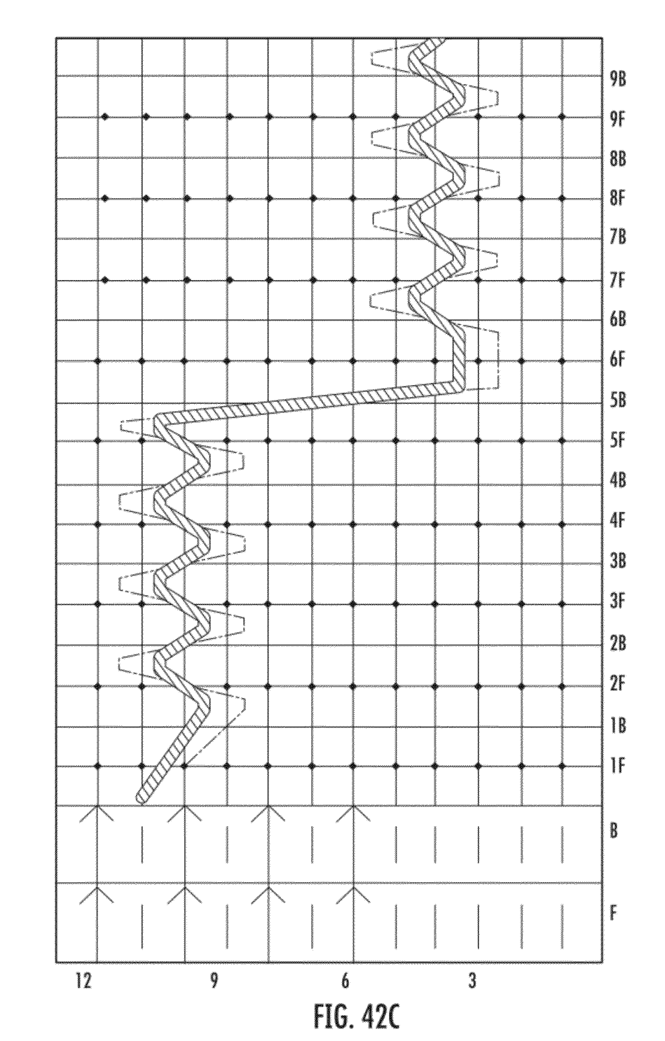

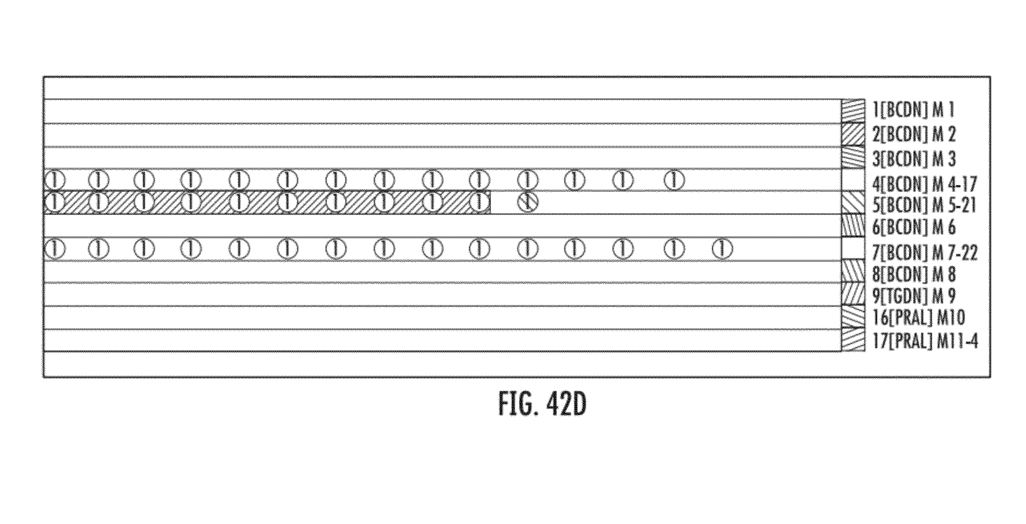

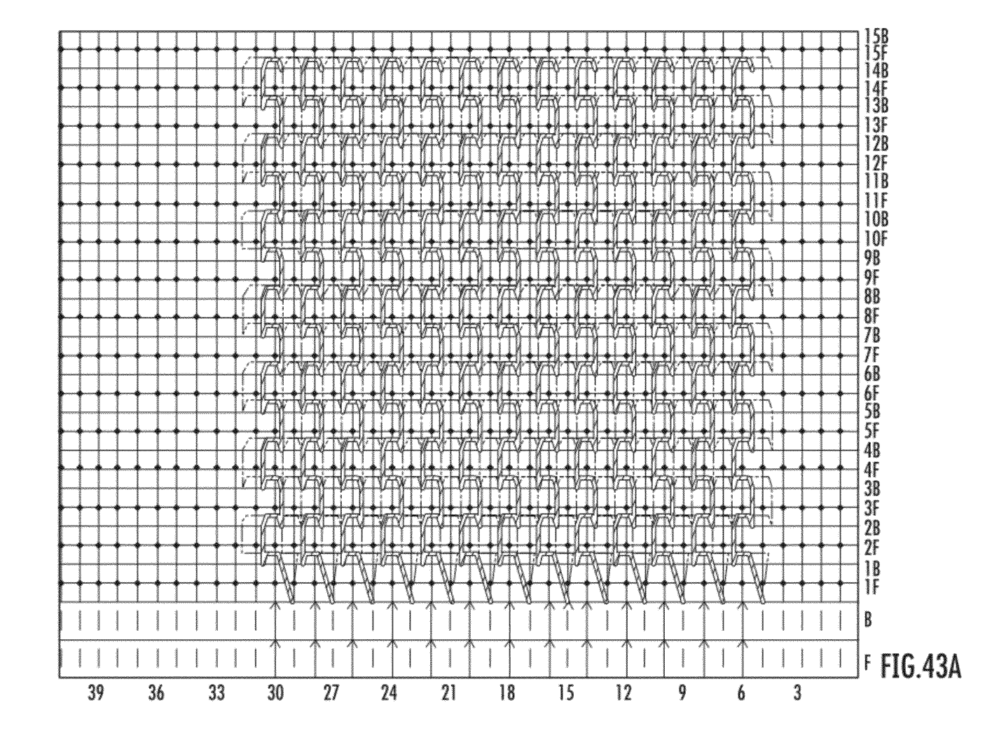

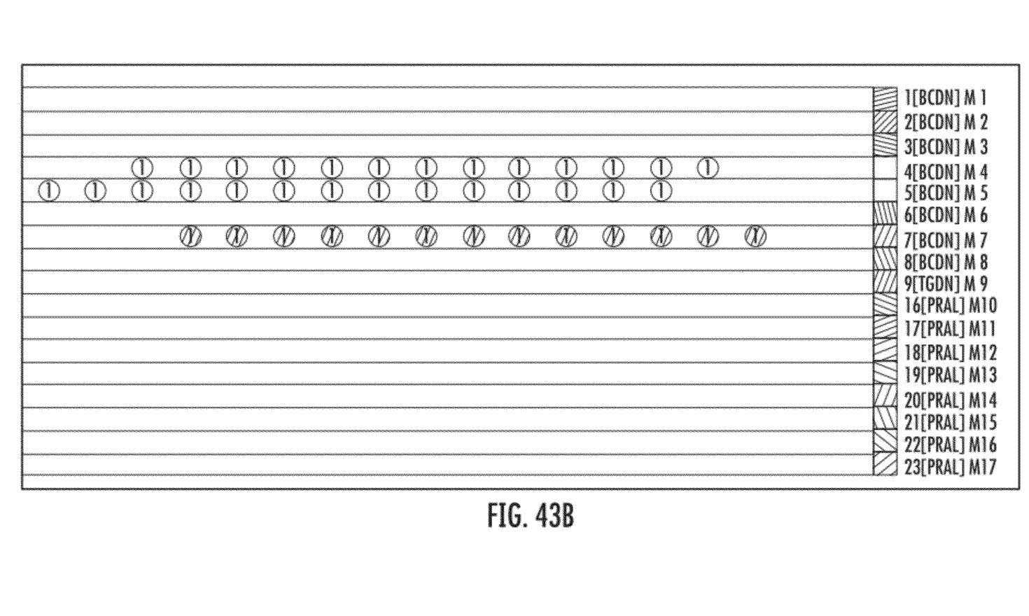

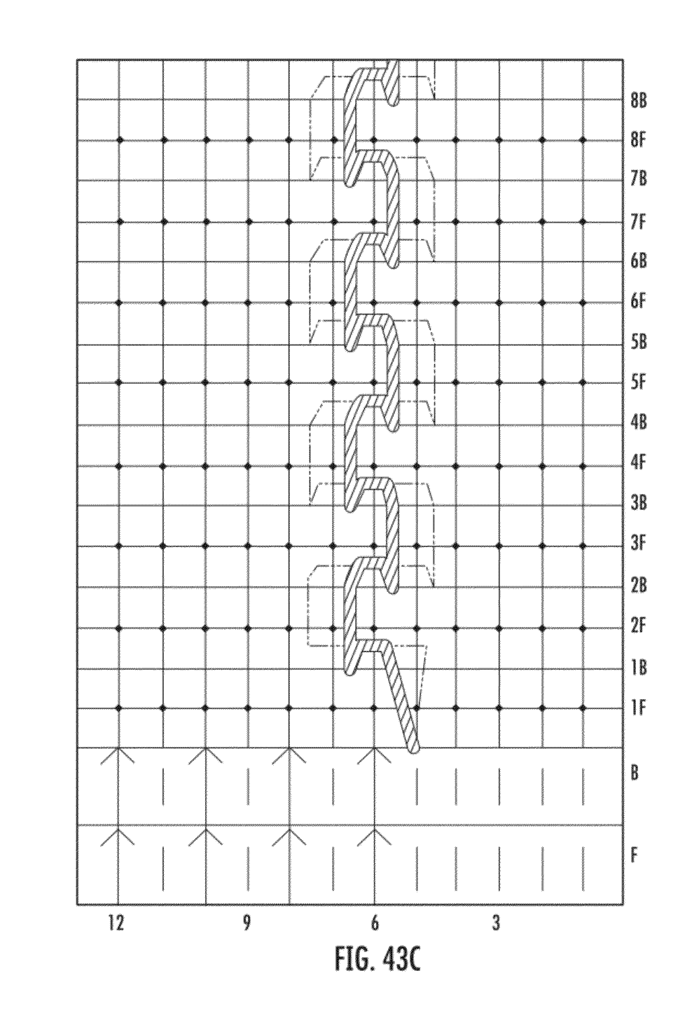

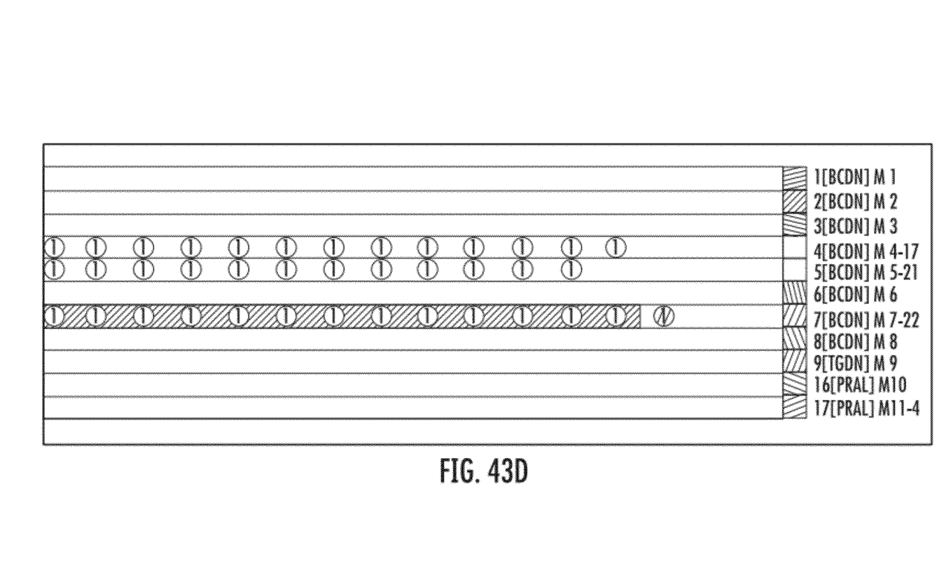

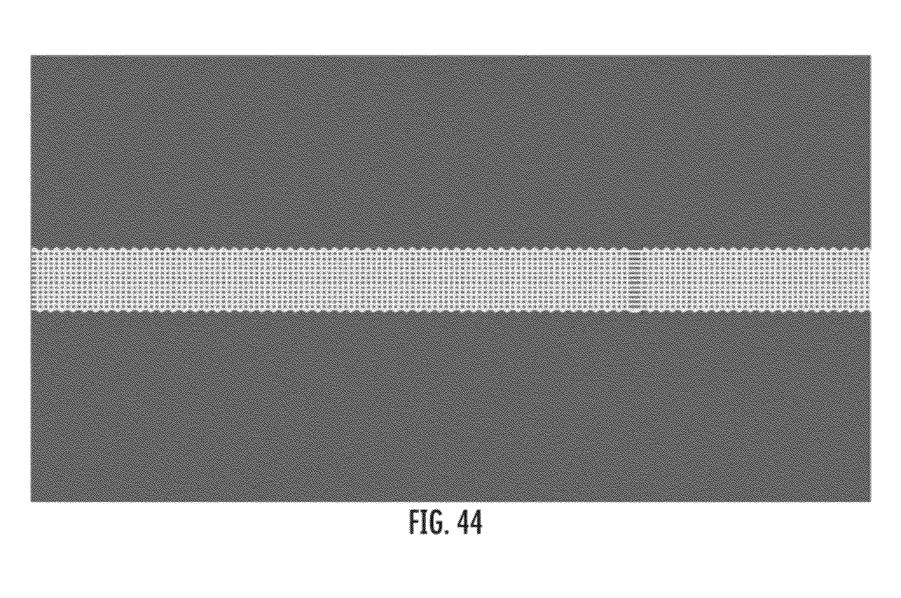

Another variation of the mesh according to an aspect of the present invention is preferably created on a raschel knitting machine such as Comez DNB/EL-800-8B set up in 10 gg needle spacing by the use of three movements as shown in pattern layout in FIGS. 40B-E: two movements in the wale direction, the vertical direction within the fabric, and one movement in the course direction, the horizontal direction of the fabric. The movements in the wale direction occurs on separate needle beds with alternate yarns; loops that occur on every course are staggered within repeat. The yarn follows a repeat pattern of 3/1-1/1-1/3-3/3-for one of the wale direction movements shown in FIGS. 41A-D and 1/1-1/3-3/3-3/1 for the other wale direction movement as shown in FIGS. 43A-D. The interlacing of the loops within the fabric allows for one yarn to be under more tension than the other under stress, locking it around the less tensioned yam; keeping the fabric from unraveling when cut. The other movement in the course direction which is shown in FIGS. 42A-D occurs in every few courses creating the porous design of the mesh. These yarns follow a repeat pattern of 9/9-9/9-7/7-9/9-7/7-9/9-7/7-9/9-7/7-9/9-1/1-1/1-3/3-1/1-3/3-1/1-3/3-1/1-3/3-1/1 for the course direction movement. The pattern simulation layout of this pattern is rendered with ComezDraw 3 software in FIG. 44 considering a yarn design made with 3 ends of Td 20/22 raw silk twisted together in the S direction to form a ply with 6 tpi and further combining three of the resulting ply with 3 tpi. The same yarn design is used for the movements occurring in the wale and course directions. The stitch density or pick count for the mesh in FIG. 44 is 34 picks per centimeter considering the total pick count for the technical front face and the technical back face of the fabric, or 17 picks per cm considering only on the face of the fabric. The operating parameters are not limited to those described in FIGS. 40B-E, but just the optimum values for the specific yarn design used for the pattern simulation layout of FIG. 44.

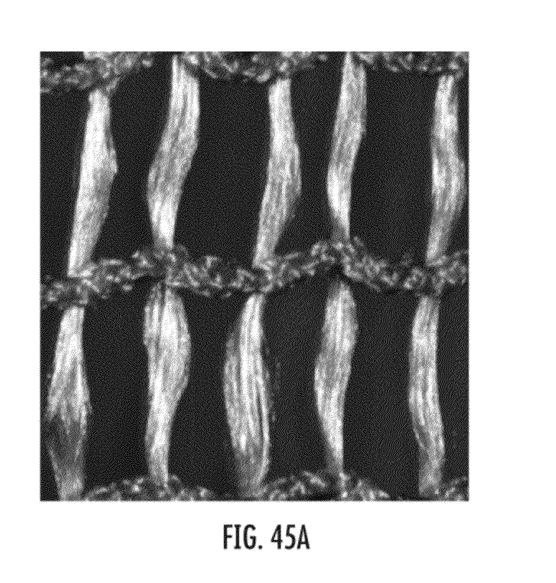

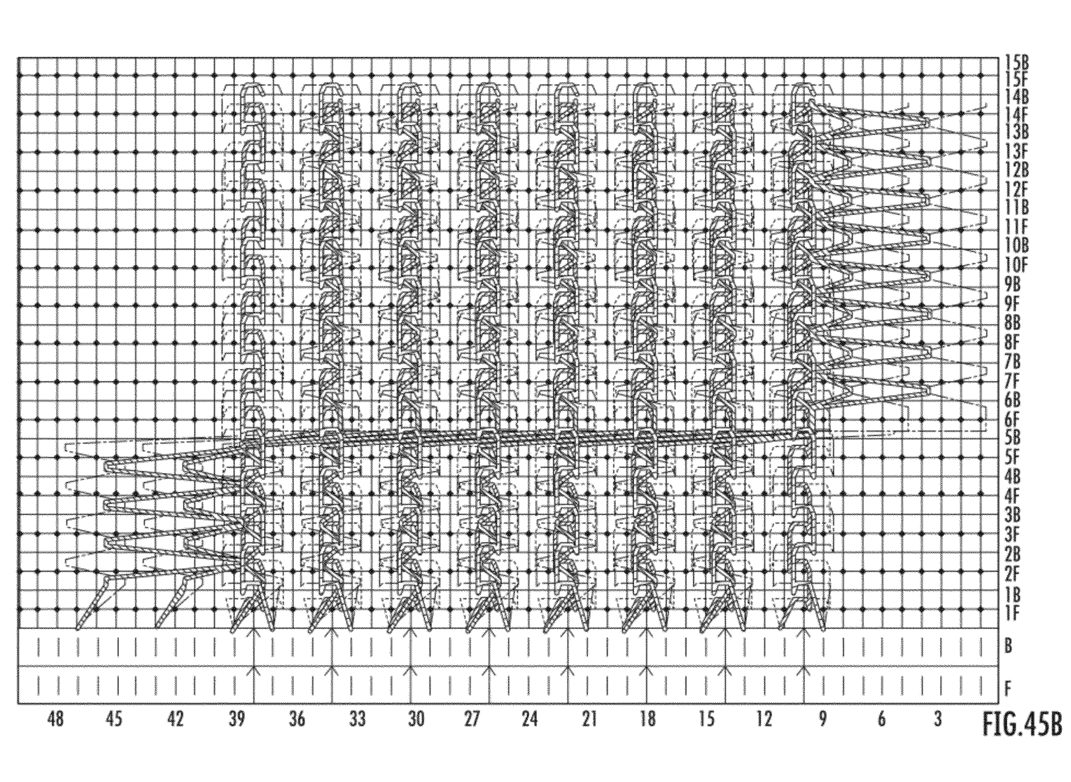

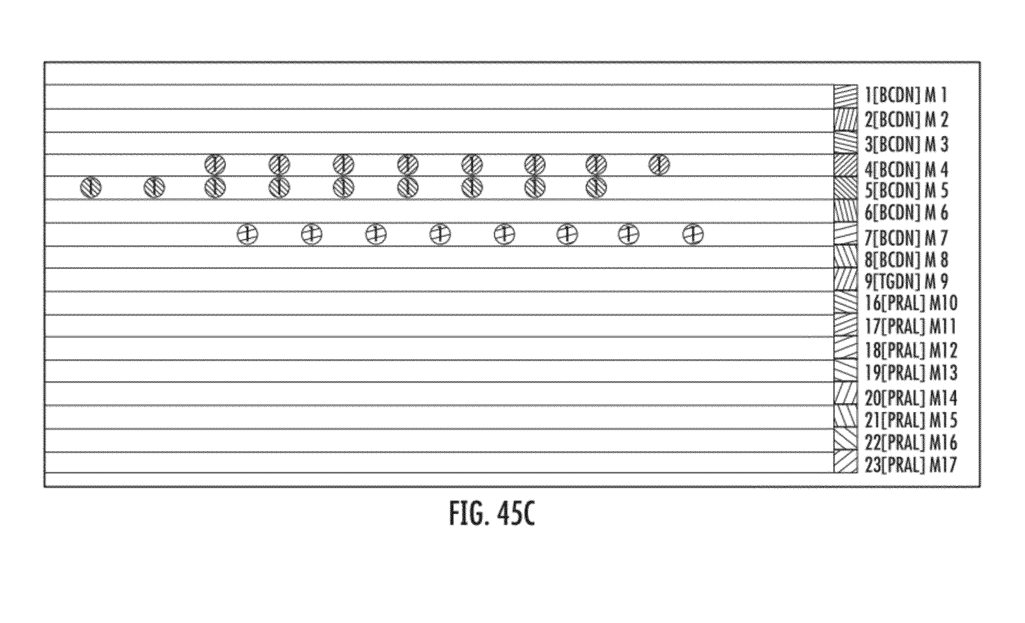

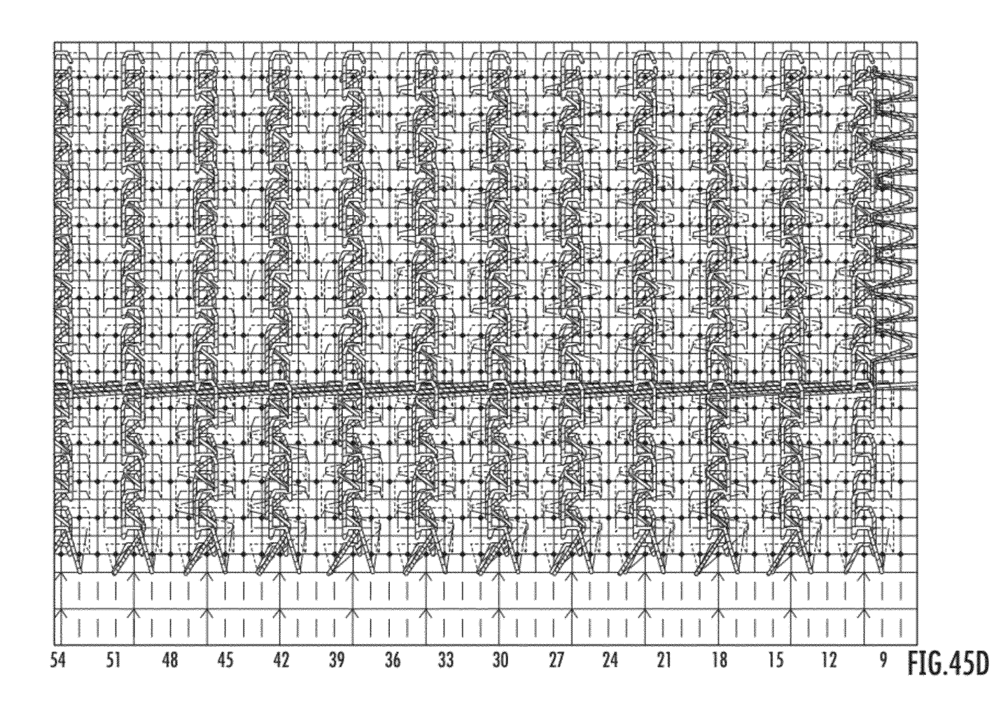

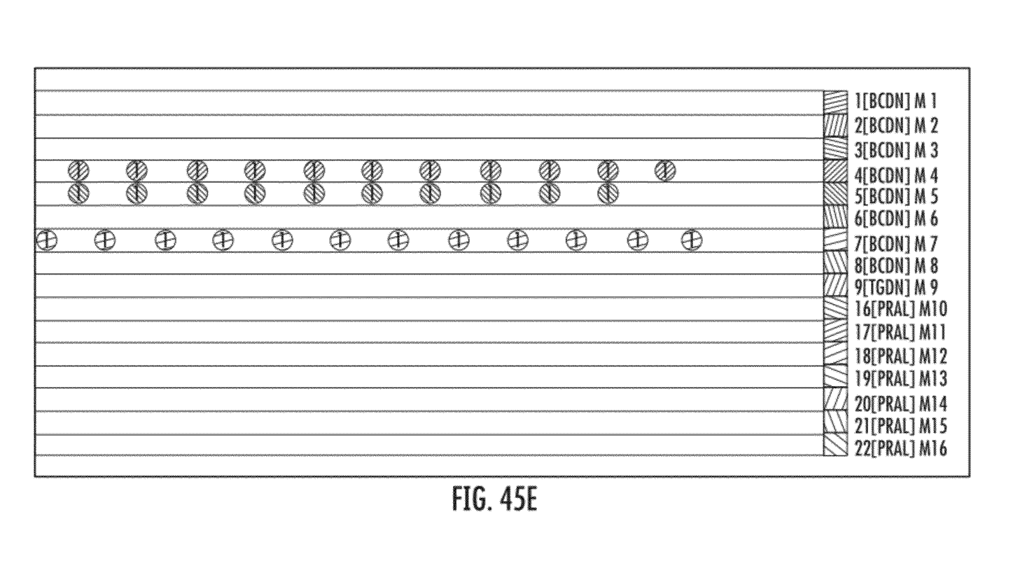

FIG. 45A is a photograph of a pattern layout for a silk-based mesh in accordance with aspects of the present invention.

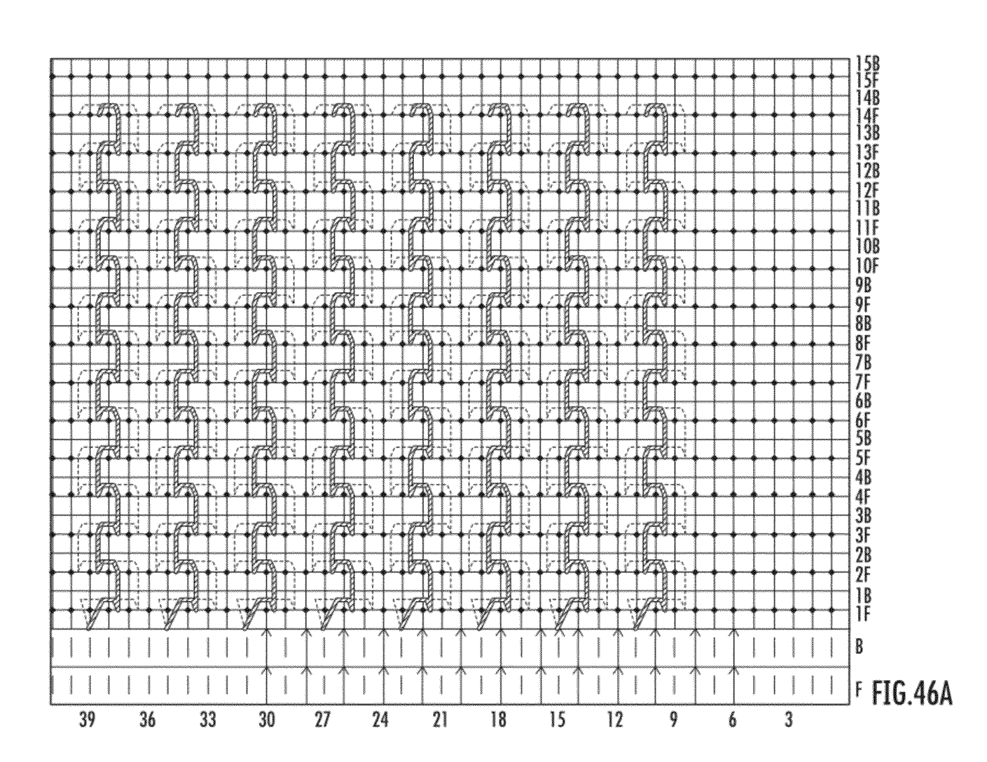

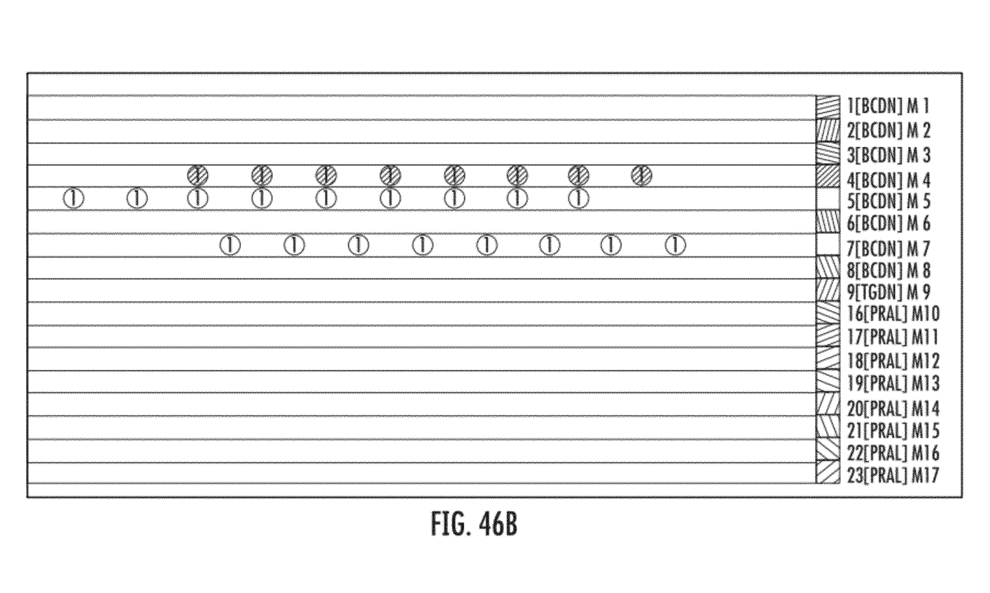

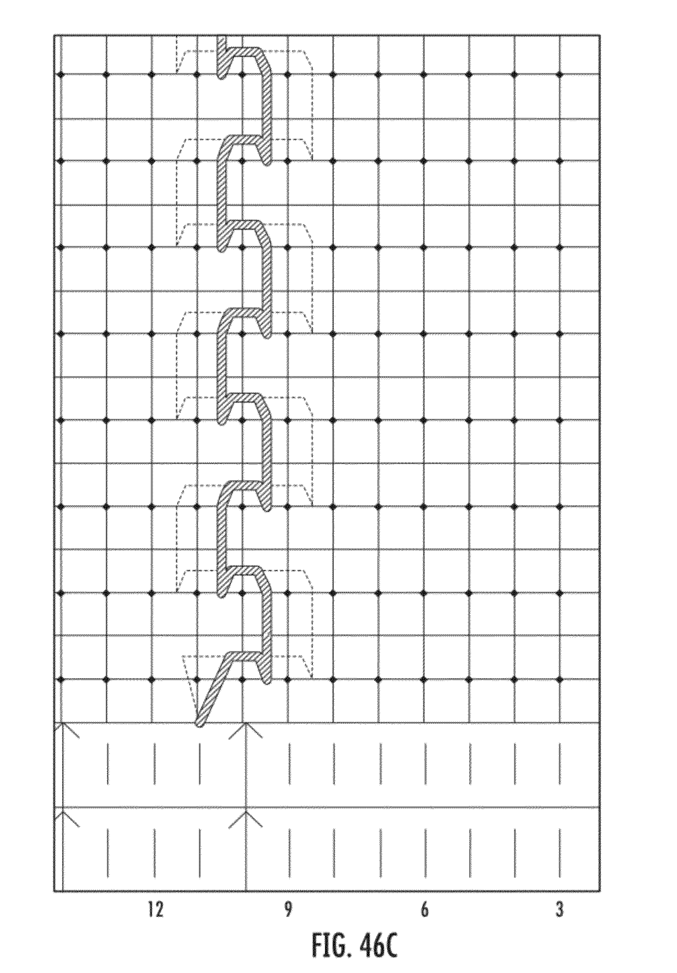

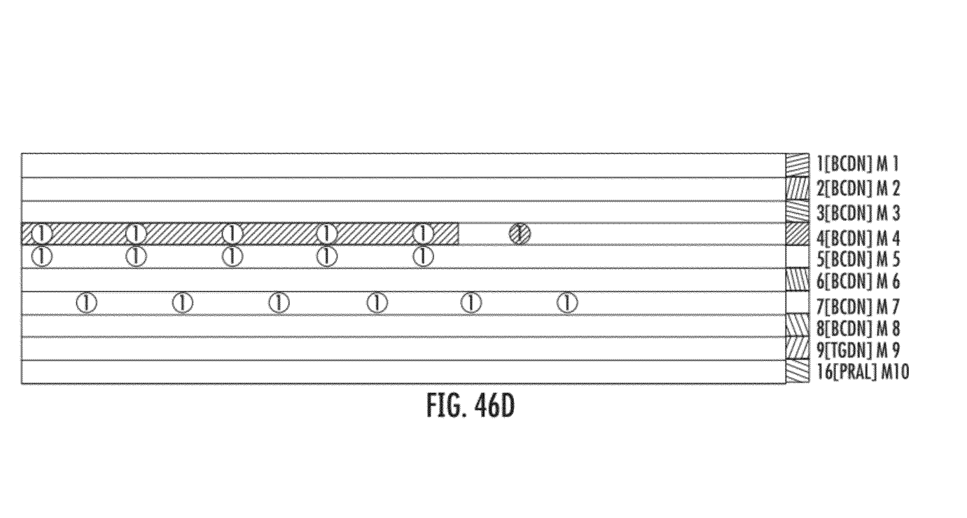

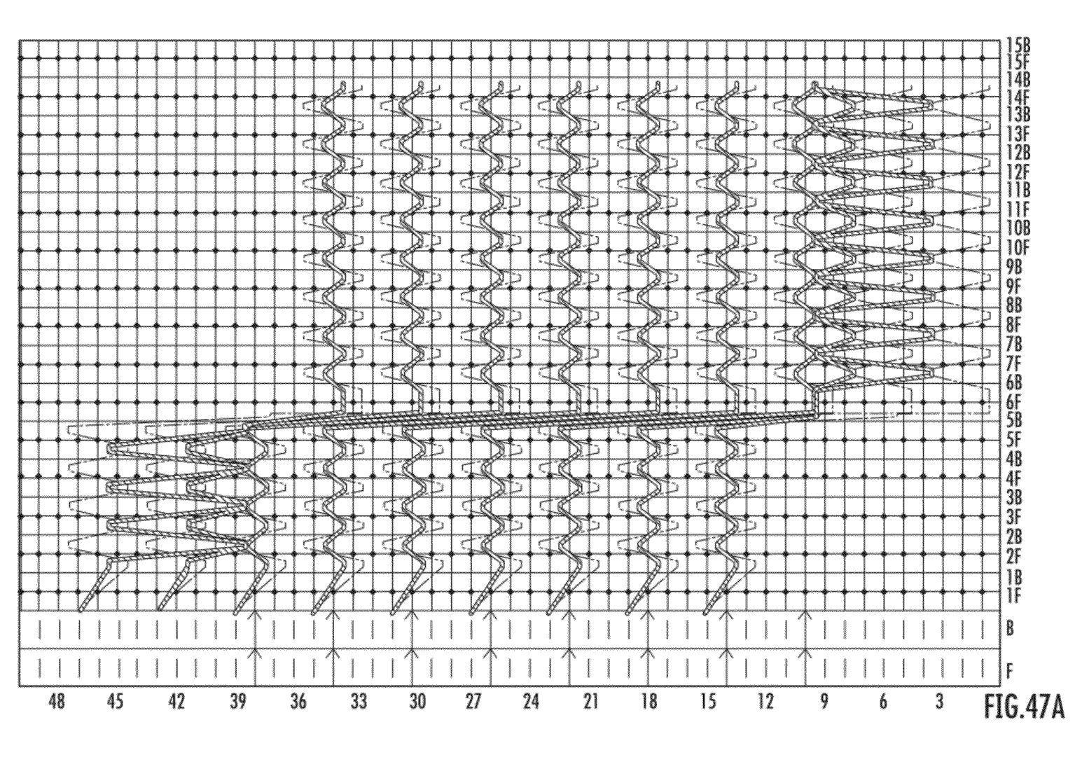

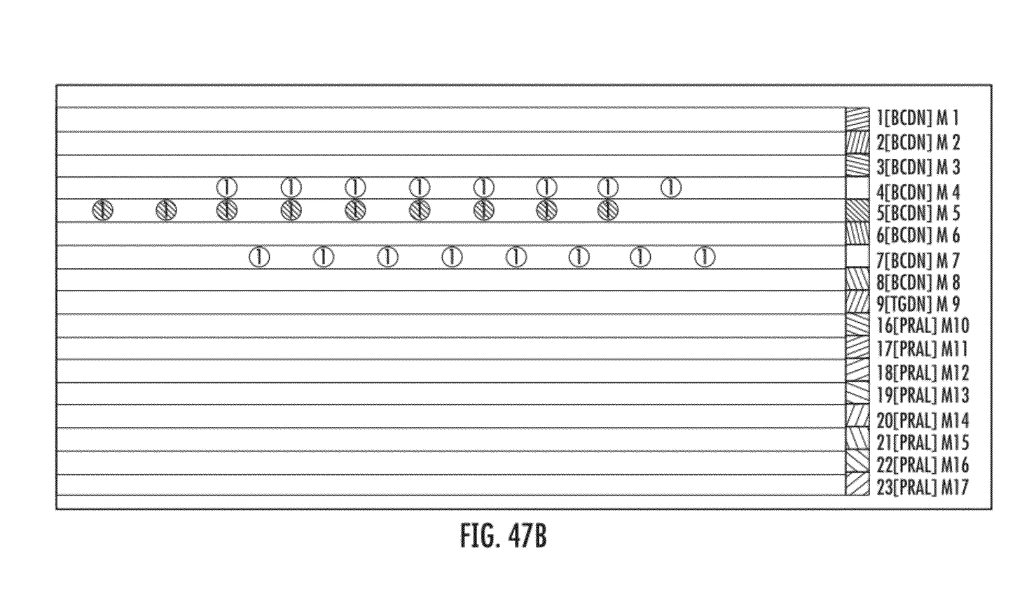

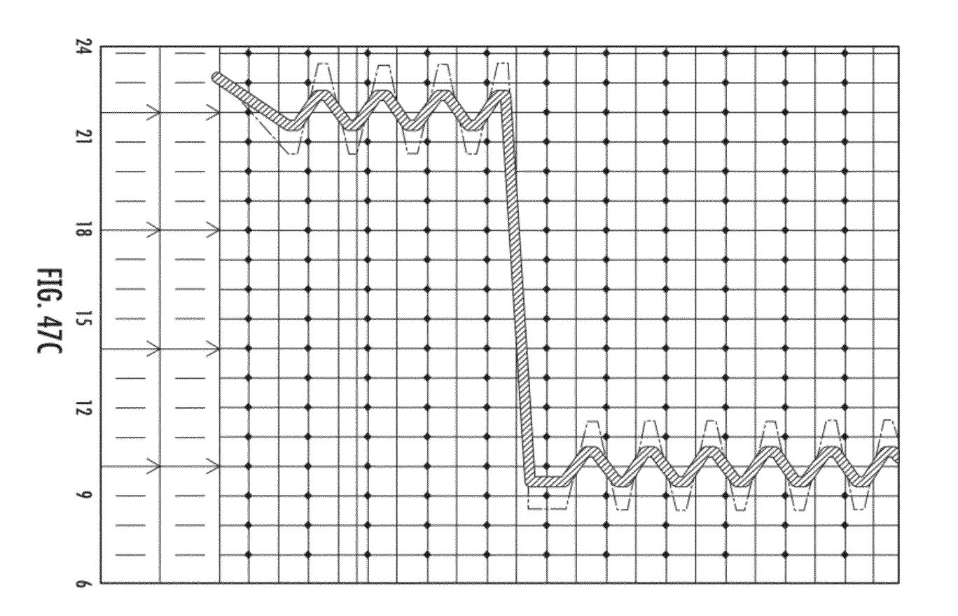

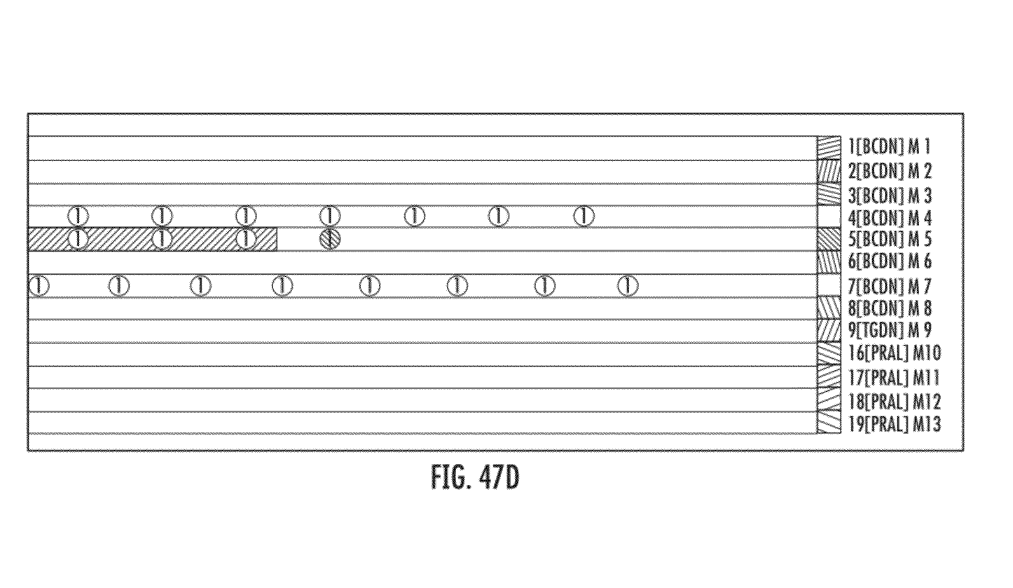

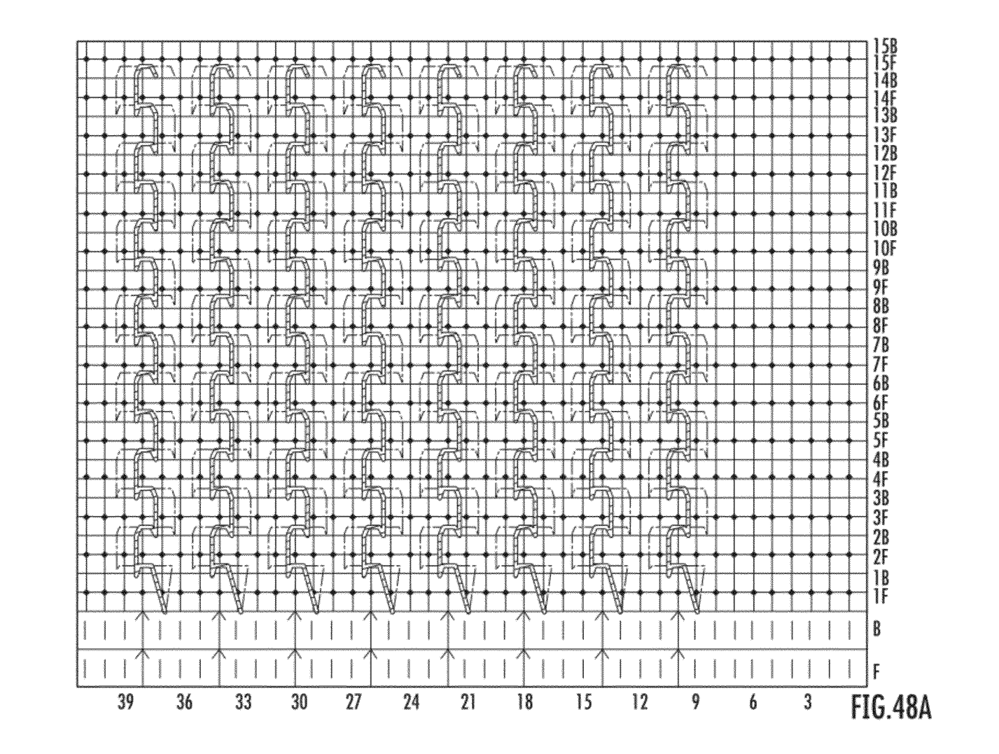

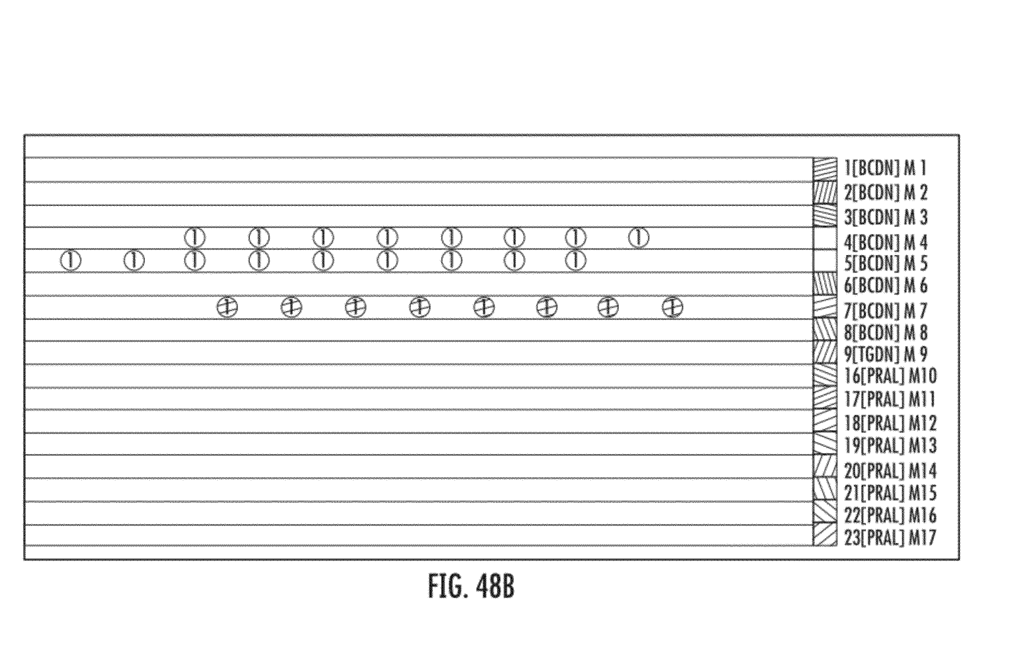

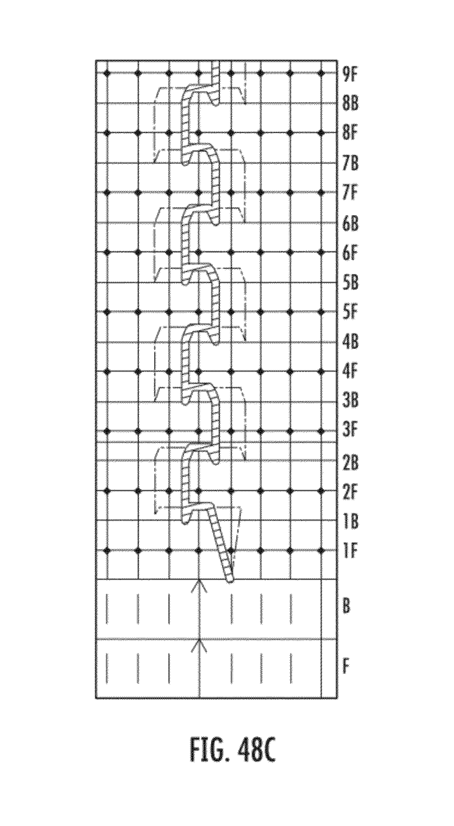

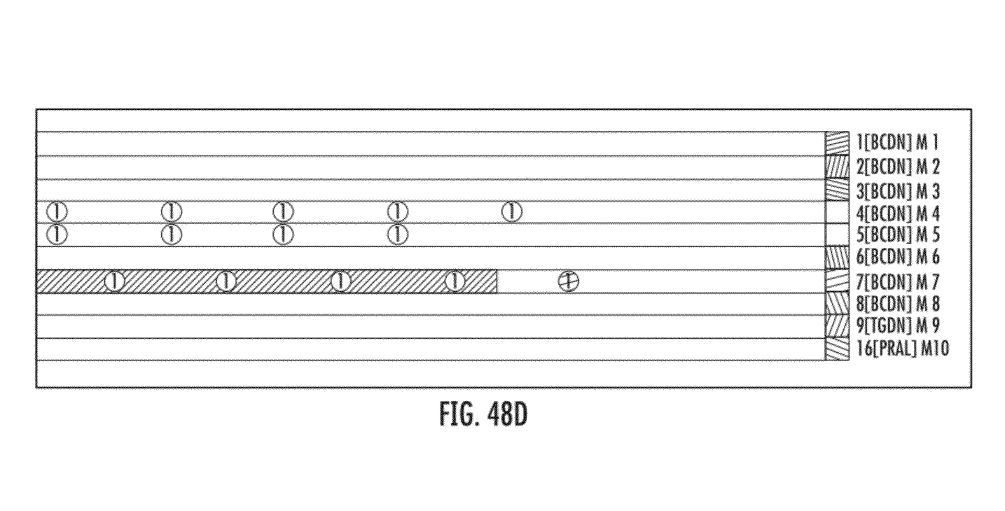

Another variation of the mesh in accordance with another aspect of the present invention is preferably created on a raschel knitting machine such as Comez DNB/EL-800-8B set up in 5 gg needle spacing by the use of three movements as shown in the pattern layout in FIGS. 45B-E: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3-for one of the wale direction movements as shown in FIGS. 46A-D and 1/1-1/3-3/3-3/1 for the other wale direction movement as shown in FIG. 48A-D. The interlacing of the loops within the fabric allows for one yarn to be under more tension than the other under stress, locking it around the less tensioned yarn; keeping the fabric from unraveling when cut. The other movement in the course direction as shown in FIG. 47A-D occurs in every few courses creating the porous design of the mesh. These yarns follow a repeat pattern of 15/15-15/15-13/13-15/15-13/13-15/15-13/13-15/15-13/13-15/15/-1/1-1/1-3/3-1/1-3/3-1/1-3/3-1/1-3/3-1/1 for the course direction movement. The pattern simulation layout of this pattern is rendered with ComezDraw 3 software in FIG. 49 considering a yarn design made with 2 ends of Td 20/22 raw silk twisted together in the S direction to form a ply with 6 tpi and further combining three of the resulting ply with 3 tpi for the two movements in the wale direction. For the movements in the course direction the yarn design is made with 3 ends of Td 20/22 raw silk twisted together in the S direction to form a ply with 6 tpi and further combining three of the resulting ply with 3 tpi. The stitch density or pick count for the mesh design in FIG. 49 is 40 picks per centimeter considering the total pick count for the technical front face and the technical back face of the fabric, or 20 picks per cm considering only on the face of the fabric. The operating parameters are not limited to these described in FIGS. 45B-E, but just the optimum values for the specific yarn design used for the pattern simulation layout of FIG. 49.

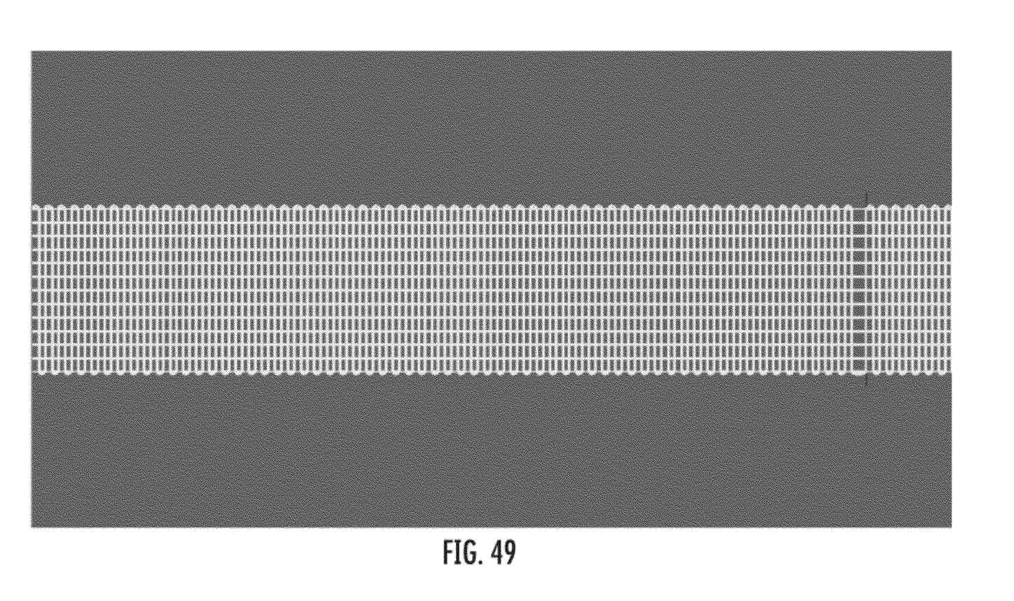

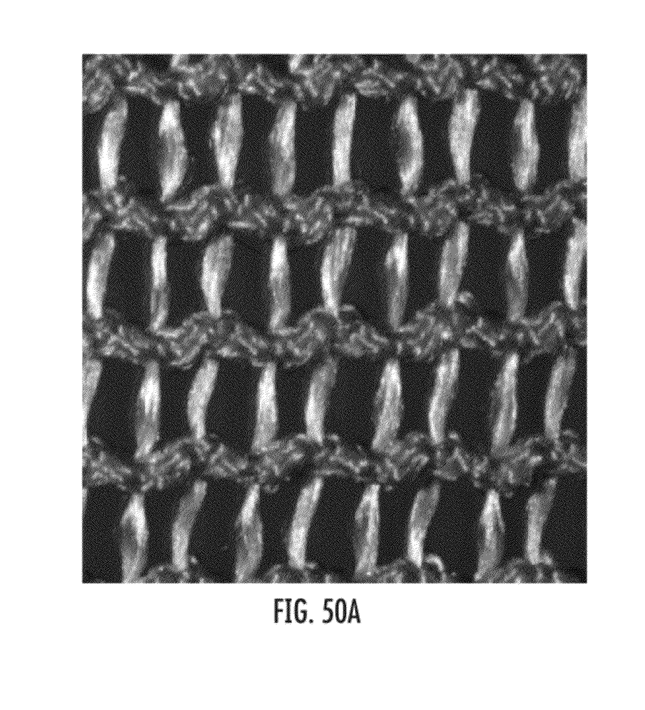

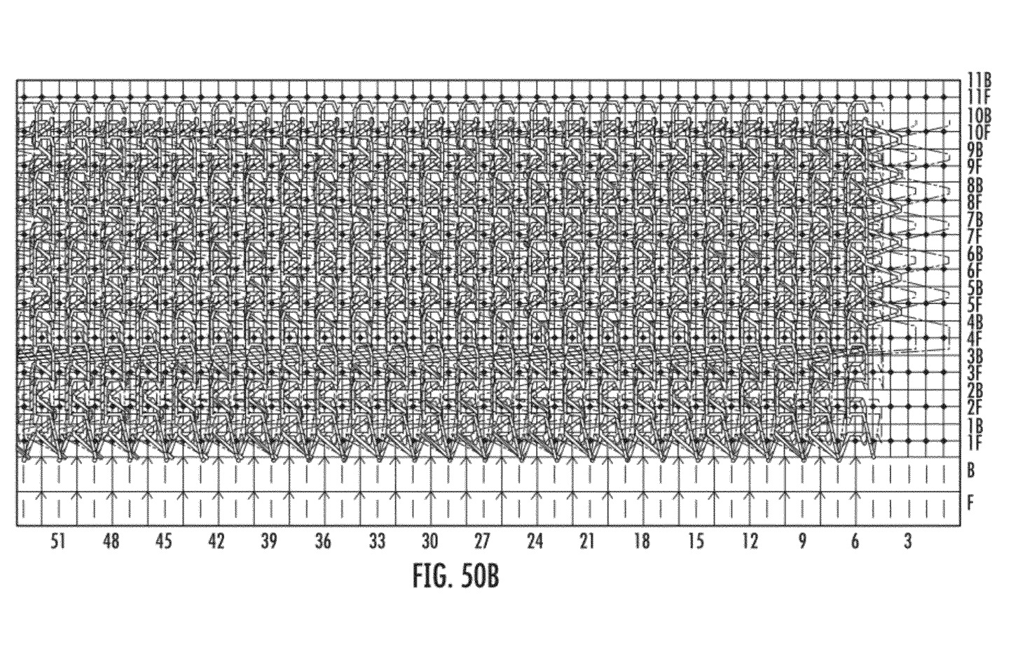

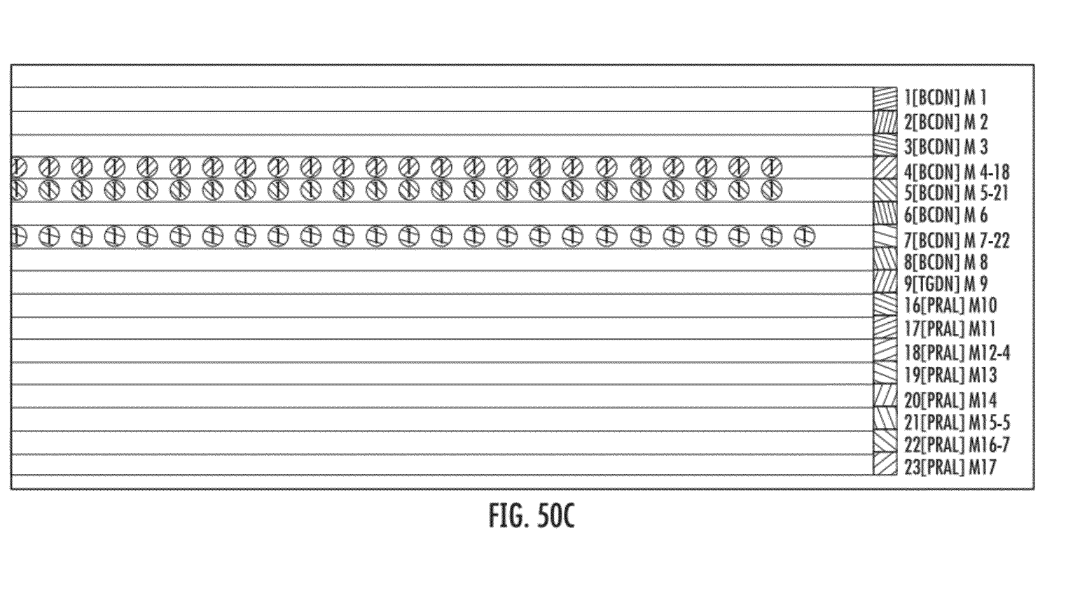

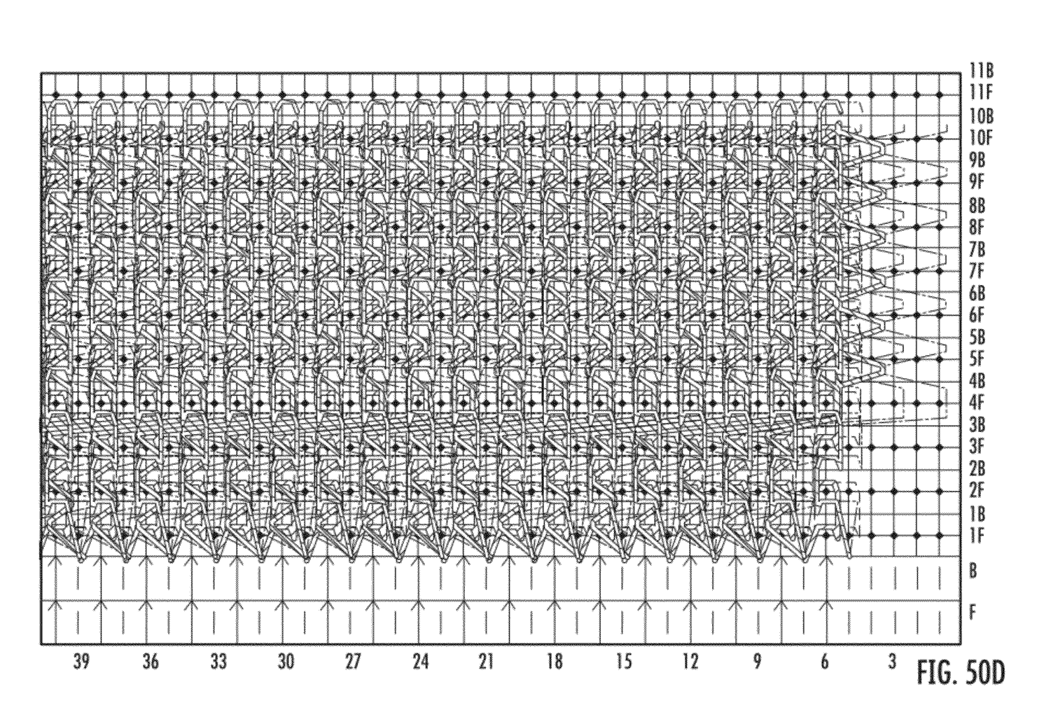

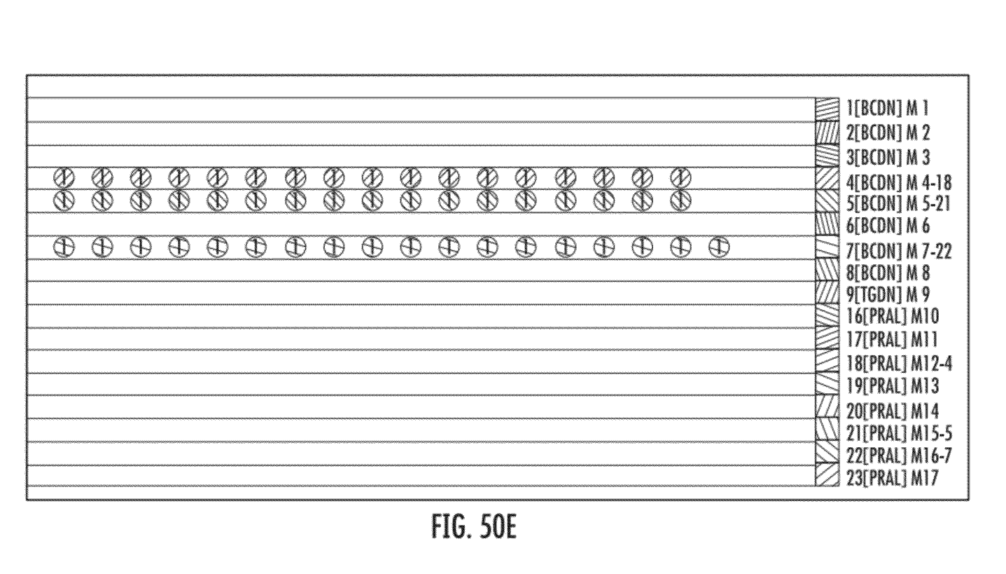

FIG. 50A is a photograph of a pattern layout for a silk-based mesh in accordance with aspects of the present invention.

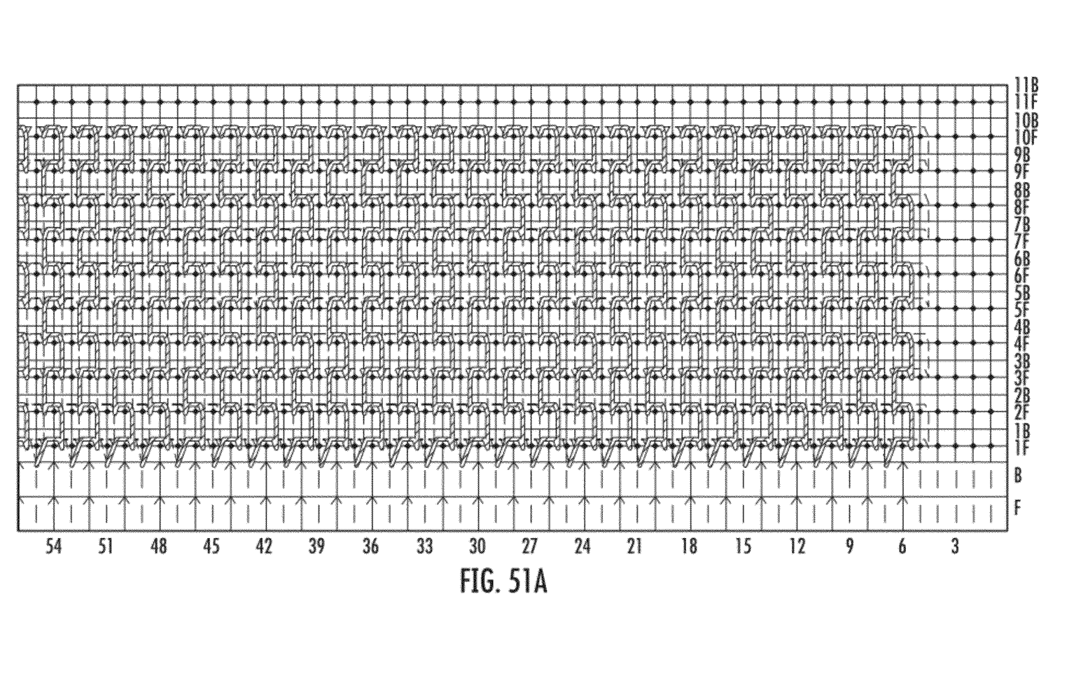

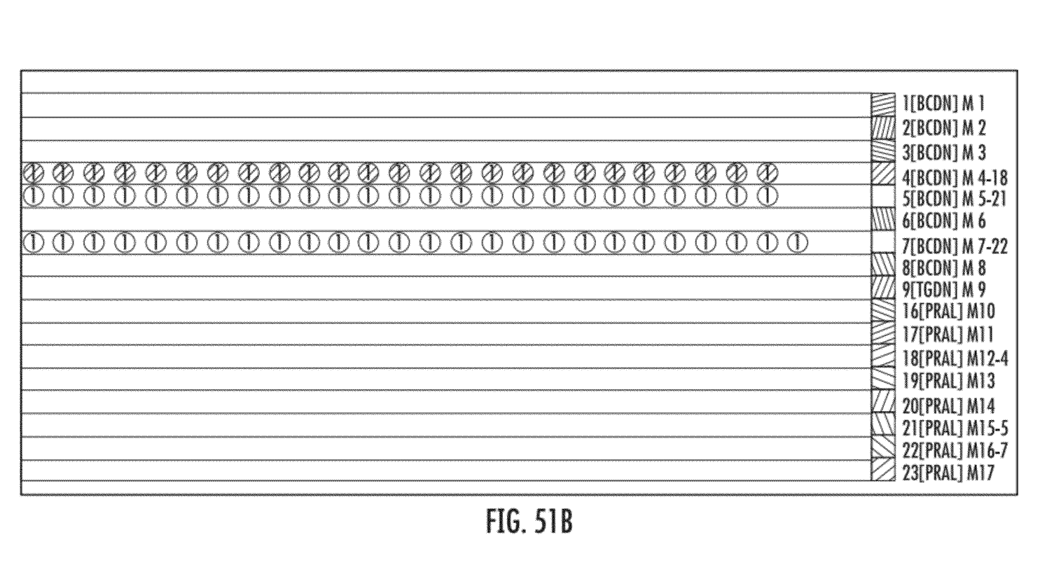

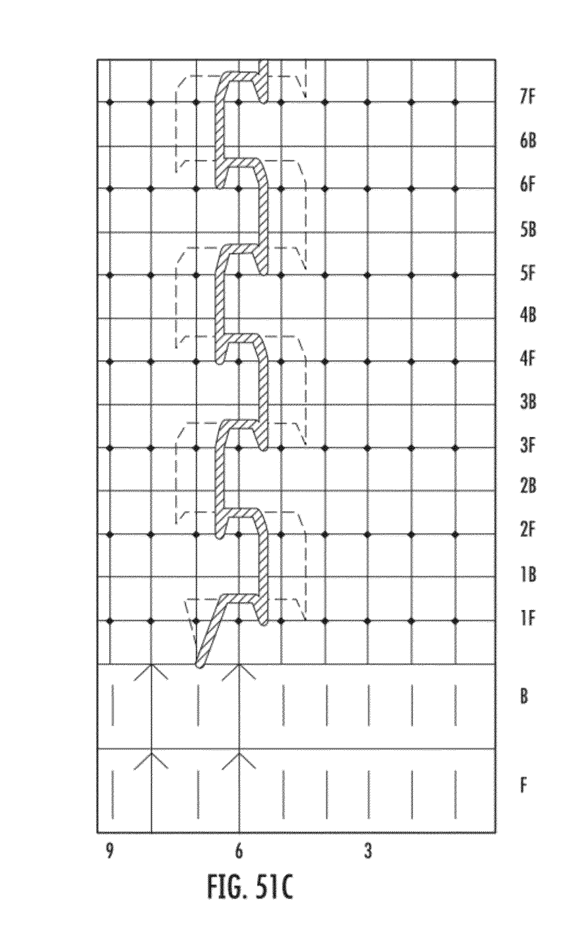

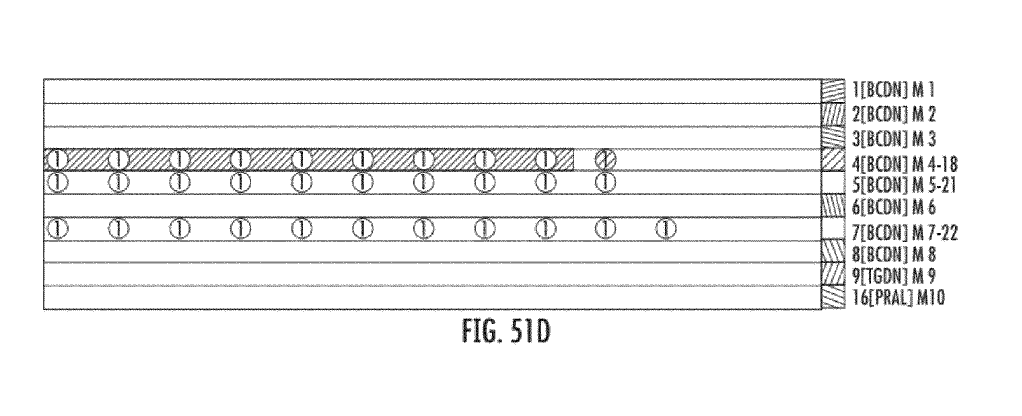

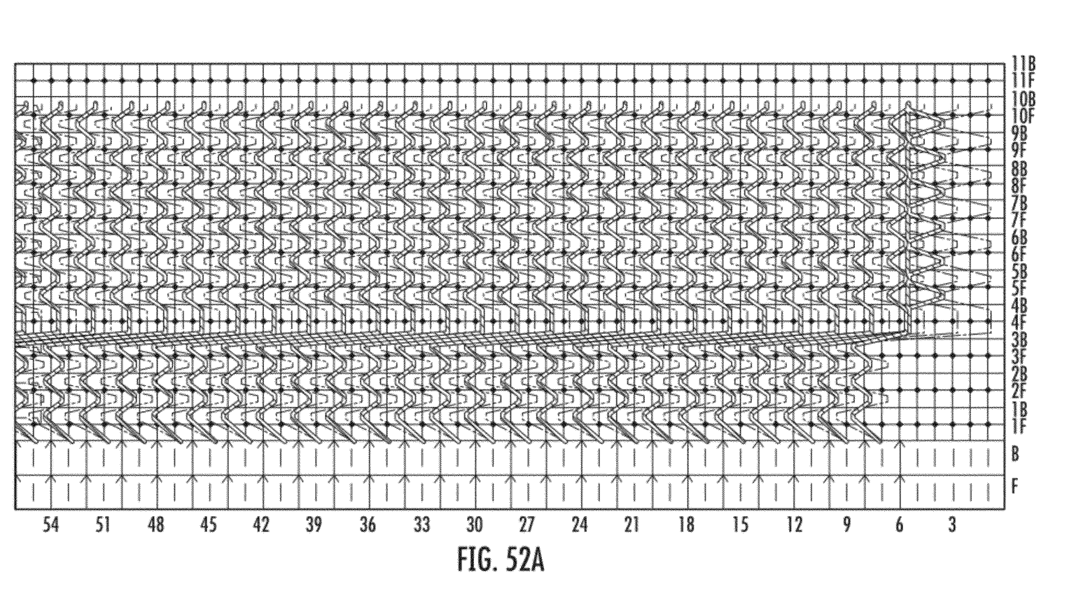

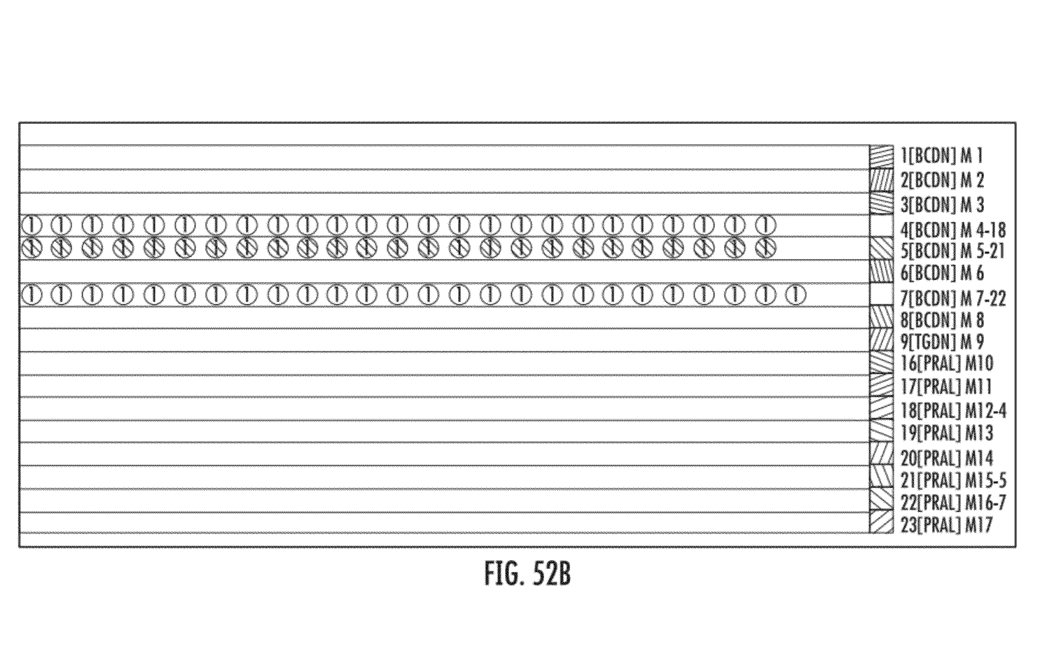

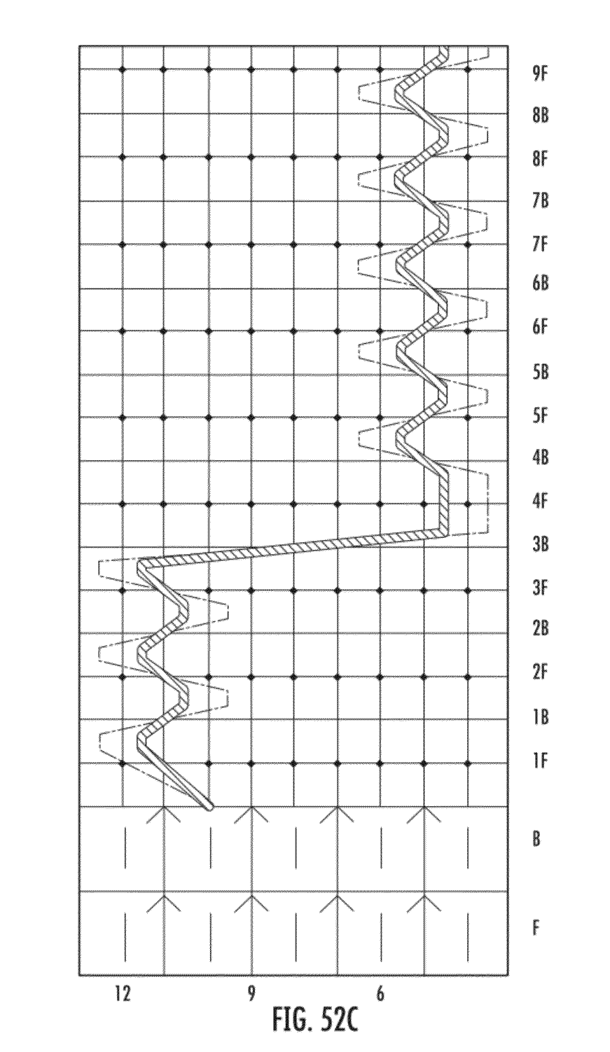

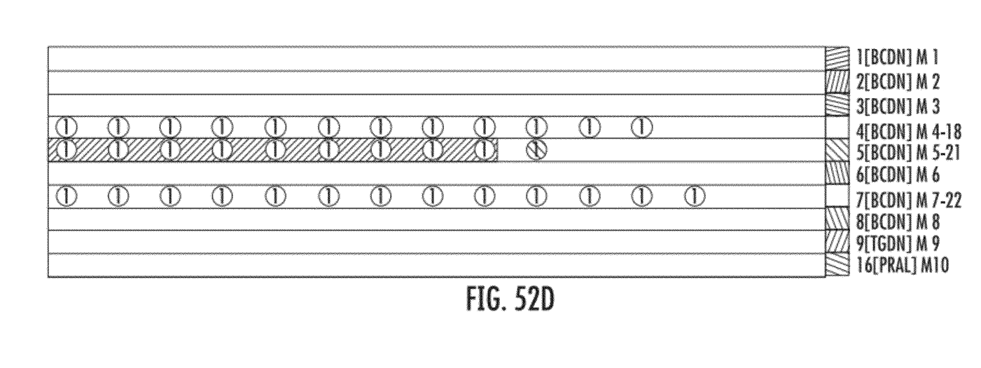

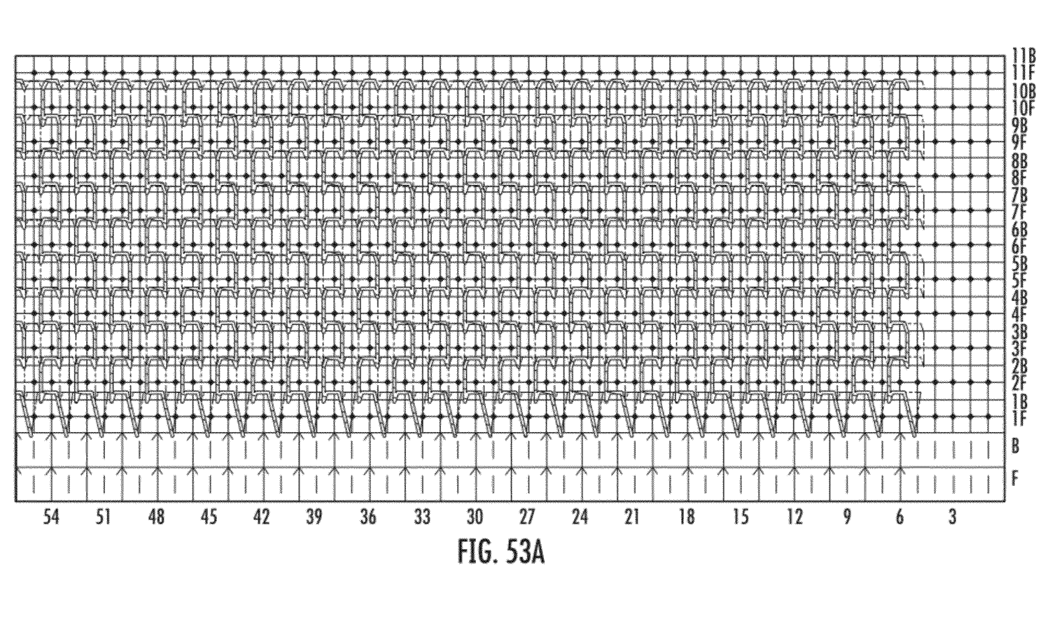

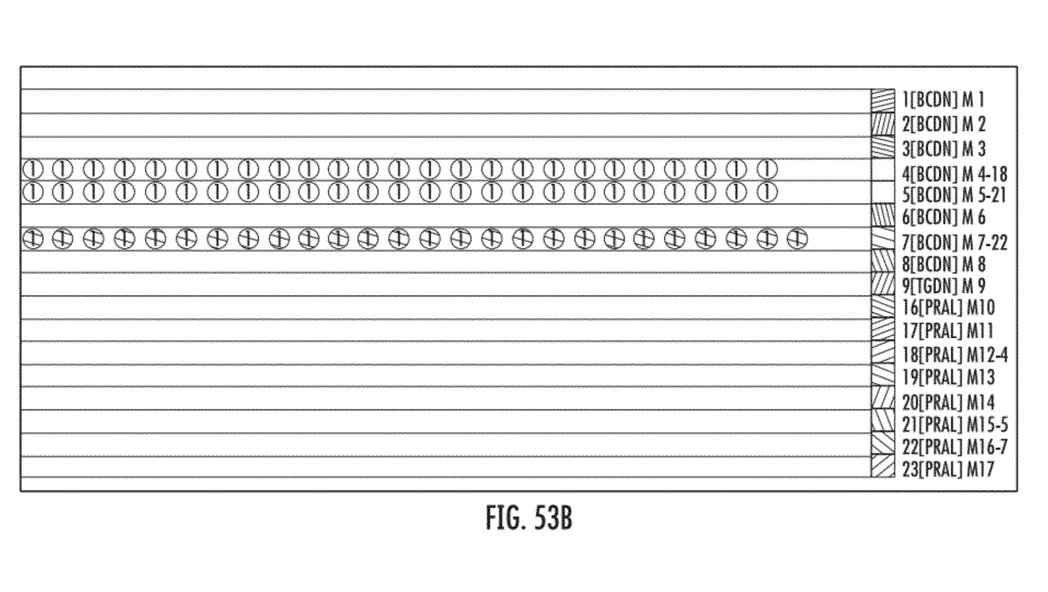

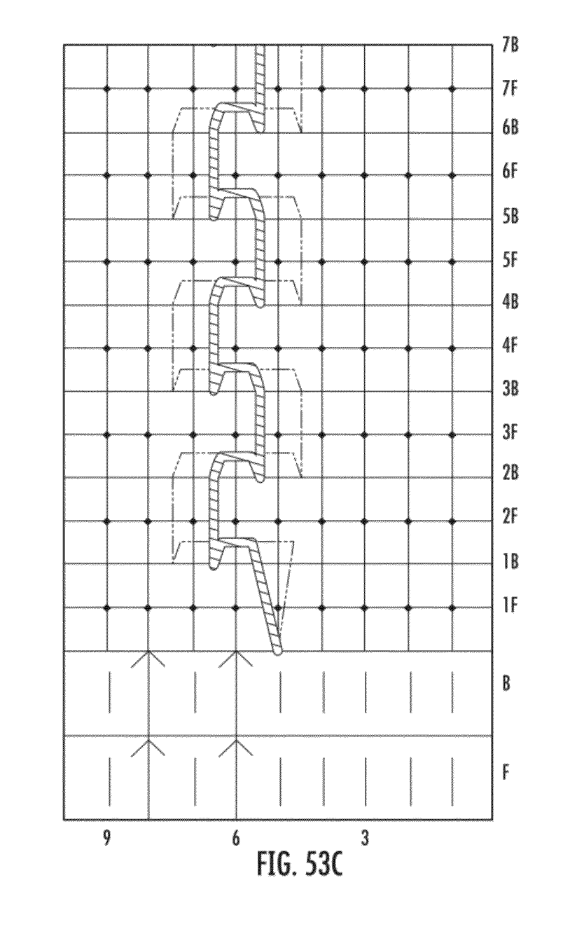

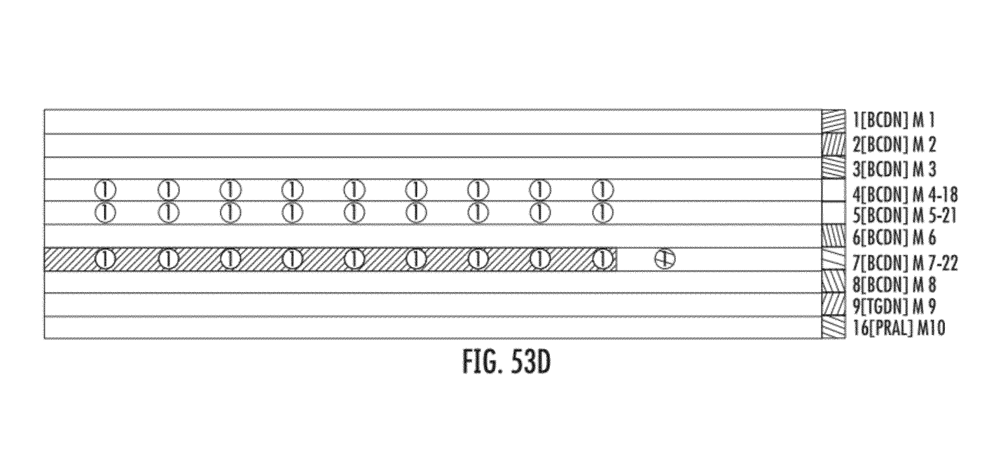

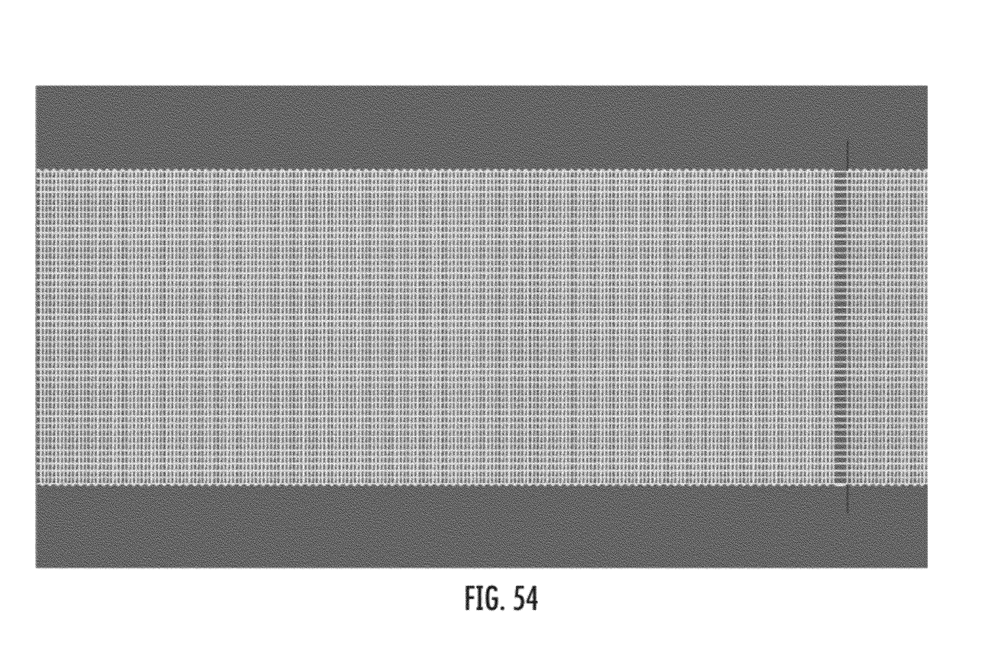

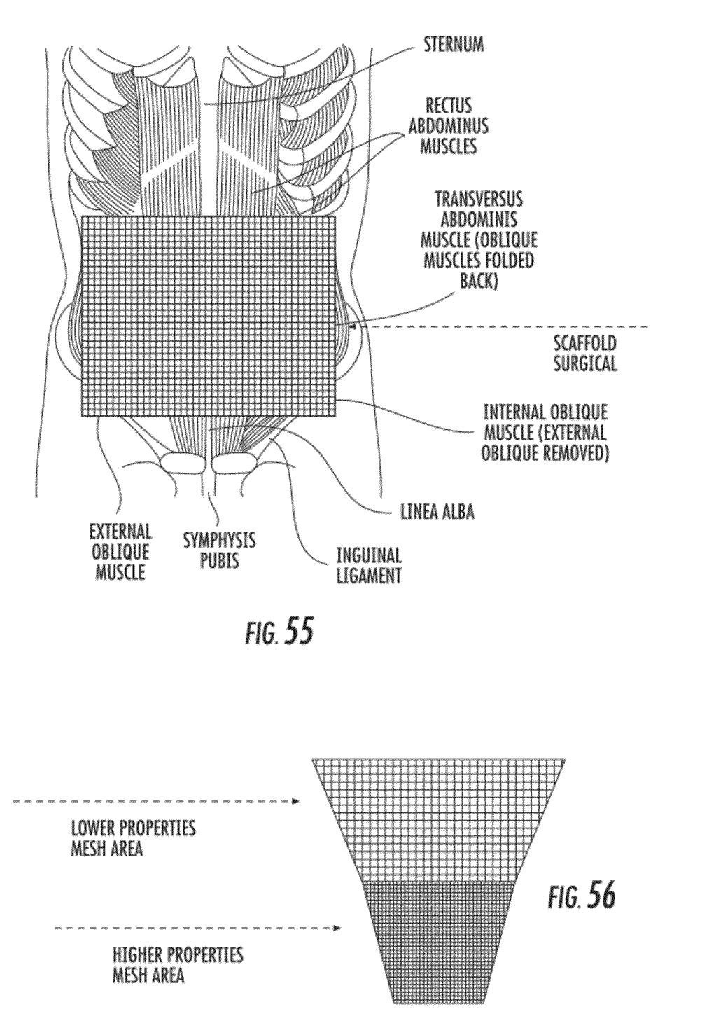

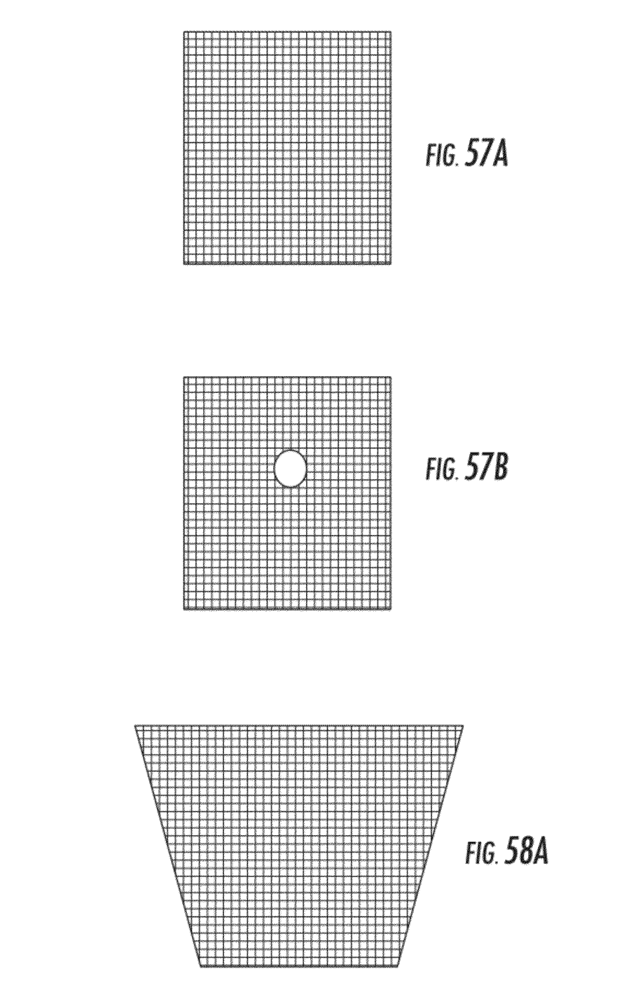

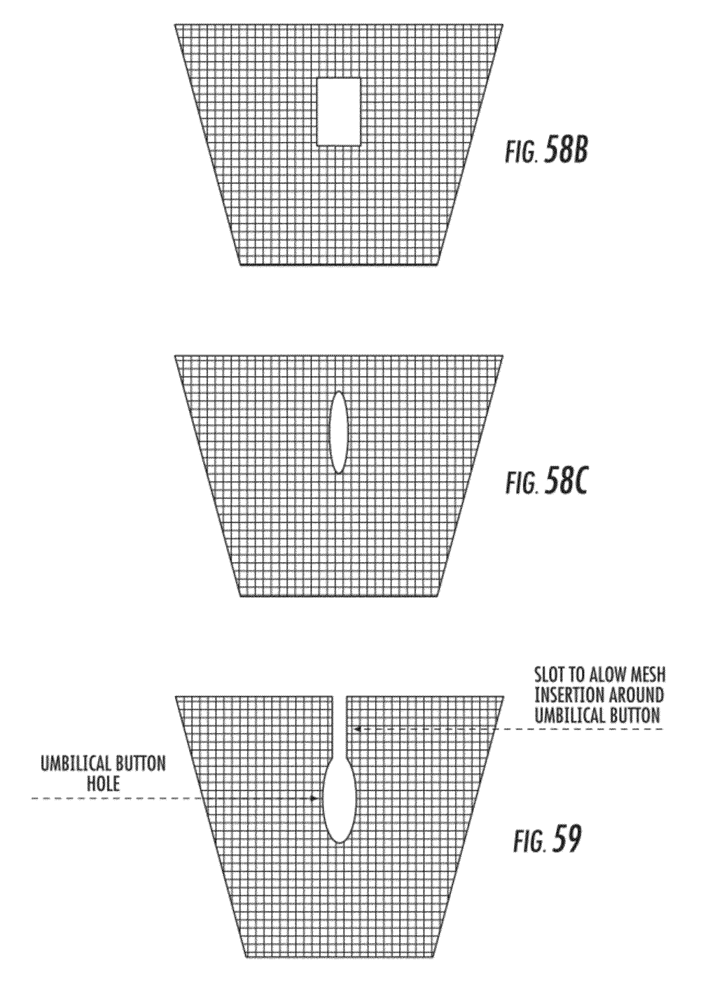

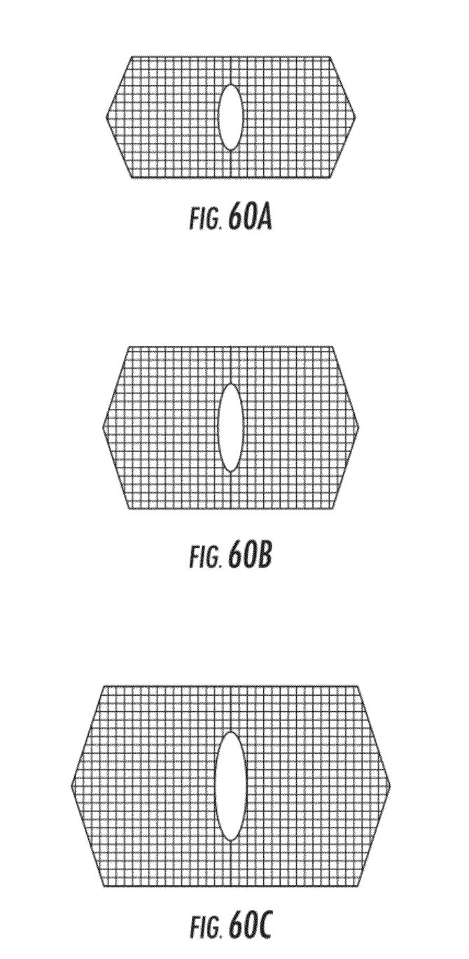

Another variation of the mesh in accordance with an aspect of the present invention may be created on a raschel knitting machine such as Comez DNB/EL-800-8B set up in 10 gg needle spacing by the use of three movements as shown in the pattern layout in FIGS. 50B-E: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3-for one of the wale direction movements shown in FIGS. 51A-D and 1/1-1/3-3/3-3/1 for the other wale direction movement shown in FIGS. 53A-D. The interlacing of the loops within the fabric allows for one yarn to be under more tension than the other under stress, locking it around the less tensioned yam; keeping the fabric from unraveling when cut. The other movement in the course direction as shown in FIGS. 52A-D occurs in every few courses creating the porous design of the mesh. These yarns follow a repeat pattern of 9/9-9/9-7/7-9/9-7/7-9/9-7/7-9/9-7/7-9/9/-1/1-1/1-3/3-1/1-3/3-1/1-3/3-1/1-3/3-1/1 for the course direction movement. The pattern simulation layout of this pattern is rendered with ComezDraw 3 software in FIG. 54 considering a yarn design made with 2 ends of Td 20/22 raw silk twisted together in the S direction to form a ply with 6 tpi and further combining three of the resulting ply with 3 tpi. The same yarn design is used for the movements occurring in the wale and course directions. The stitch density or pick count for the mesh in FIG. 54 is 40 picks per centimeter considering the total picks count for the technical front and the technical back of the fabric, or 20 picks per cm considering only on the face of the fabric. The operating parameters are not limited to the those described in FIGS. 50B-E, but just the optimum values for the specific yarn design used for the pattern simulation layout of FIG. 54.

In embodiments employing silk yarn, the silk yarn may be twisted from yarn made by 20-22 denier raw silk fibers approximately 40 to 60 μm in diameter. Preferably, raw silk fibers ranging from 10 to 30 deniers may be employed; however any fiber diameters that will allow the device to provide sufficient strength are acceptable. Advantageously, a constant yarn size may maximize the uniformity of the surgical mesh mechanical properties, e.g. stiffness, elongation, etc., physical and/or biological properties within each region. However, the yarn size may be varied in sections of the mesh in order to achieve different mechanical, physical and/or biological characteristics in the preferred mesh locations. Factors that may be influenced by the size of the yarn include, but are not limited to: ultimate tensile strength (UTS); yield strength, i.e. the point at which yarn is permanently deformed; percent elongation; fatigue and dynamic laxity (creep); bioresorption rate; and transfer of cell/nutrients into and out of the mesh.

The knit patterns illustrated in FIGS. 29A, 34A, 40A, 45A and 50A respectively, may be knit to any width depending upon the knitting machine and could be knitted with any of the gauges available with the various crochet machines or warp knitting machines. Table 1B outlines the fabric widths that may be achieved using a different numbers of needles on different gauge machines. It is understood that the dimensions in Table 1B are approximate due to the shrink factor of the knitted fabric which depends on stitch design, stitch density, and yarn size used.

TABLE 1B

| | |
|---|---|
| Needle Count | Knitting Width (mm) |

| Gauge | From | To | From | To |
|---|---|---|---|---|
| 48 | 2 | 5656 | 0.53 | 2997.68 |
| 24 | 2 | 2826 | 1.06 | 2995.56 |
| 20 | 2 | 2358 | 1.27 | 2994.66 |
| 18 | 2 | 2123 | 1.41 | 2993.43 |
| 16 | 2 | 1882 | 1.59 | 2992.38 |
| 14 | 2 | 1653 | 1.81 | 2991.93 |
| 12 | 2 | 1411 | 2.12 | 2991.32 |
| 10 | 2 | 1177 | 2.54 | 2989.58 |
| 5 | 2 | 586 | 5.08 | 2976.88 |

Mesh or scaffold designs in accordance with aspects of the present invention may be knitted on a fine gauge crochet knitting machine. Crochet machines capable of manufacturing the mesh in accordance with aspects of the present invention include, but are not limited to, those provided by: Changde Textile Machinery Co., Ltd.; Comez; China Textile Machinery Co., Ltd.; Huibang Machine; Jakob Muller AG; Jingwei Textile Machinery Co., Ltd.; Zhejiang Jingyi Textile Machinery Co., Ltd.; Dongguan Kyang the Delicate Machine Co., Ltd.; Karl Mayer; Sanfang Machine; Sino Techfull; Suzhou Huilong Textile Machinary Co., Ltd.; Taiwan Giu Chun Ind. Co., Ltd.; Zhangjiagang Victor Textile; Liba; Lucas; Muller Frick; and Texma.

Mesh or scaffold designs in accordance with aspects of the present invention may be knitted on a fine gauge warp knitting machine. Warp knitting machines capable of manufacturing the mesh in accordance with aspects of the present invention include, but are not limited to, those provided by: Comez; Diba; Jingwei Textile Machinery; Liba; Lucas; Karl Mayer; Muller Frick; Runyuan Warp Knitting; Taiwan Giu Chun Ind.; Fujian Xingang Textile Machinery; and Yuejian Group.

Mesh or scaffold designs in accordance with aspects of the present invention may be knitted on a fine gauge flat bed knitting machine. Flat bed machines capable of manufacturing the mesh in accordance with aspects of the present invention include, but are not limited to, those provided by: Around Star; Boosan; Cixing Textile Machine; Fengshen; Flying Tiger Machinery; Fujian Hongqi; G & P; Gorteks; Jinlong; JP; Jy Leh; Kauo Heng Co., Ltd.; Matsuya; Nan Sing Machinery Limited; Nantong Sansi Instrument; Shima Seiki; Nantong Tianyuan; and Ningbo Yuren Knitting.

A test method was developed to check the cutability of the surgical mesh formed according to aspects of the present invention. In the test method, the surgical mesh evaluated according to the number of were needed to cut the mesh with surgical scissors. The mesh was found to cut excellently because it took one scissor stroke to cut through it. The mesh was also cut diagonally and in circular patterns to determine how easily the mesh unraveled and how mush it unraveled once cut. The mesh did not unravel more than one mode after being cut in both directions. To determine further if the mesh would unravel, a suture, was passed through the closest pore from the cut edge, and pulled. This manipulation did not unravel the mesh. Thus, the surgical mesh is easy to cut and does not unravel after manipulation.

Embodiments may be processed with a surface treatment, which increases material hydrophilicity, biocompatibility, physical, and mechanical properties such as handling for ease of cutting and graft pull-through, as well as anti-microbial and anti-fungal coatings. Specific examples of surface treatments include, but are not limited to:
plasma modification
protein such as but not limited to fibronectin, denatured collagen or gelatin, collagen gels and hydrophobin by covalent link or other chemical or physical method
peptides with hydrophilic and a hydrophobic end
peptides contain one silk-binding sequence and one biologically active sequence—biodegradable cellulose
surface sulfonation
ozone gas treatment
physically bound and chemically stabilized peptides
DNA/RNA aptamers
Peptide Nucleic Acids
Avimers
modified and unmodified polysaccharide coatings
carbohydrate coating
anti-microbial coatings
anti-fungal coatings
phosphorylcholine coatings

A method to evaluate the ease of delivery through a cannula was done to make sure the surgical mesh could be used laparoscopically. Various lengths were rolled up and pushed through two different standard sized cannulas using surgical graspers. The mesh was then evaluated to determine if there was any damage done to the mesh. The mesh that was put through the cannulas was found to have slight distortion to the corner that was held by the grasper. The 16 cm and 18 cm lengths of mesh that were rolled up and pushed through the 8 mm cannula had minimal fraying and one distorted pore, respectively. It was also found that no damage was done to the cannula or septum in any of the tests. It was found that appropriately sized surgical mesh will successfully pass through a laparoscopic cannula without damage, enabling its effective use during laparoscopic procedures.

A surgical mesh device according to aspects of the present invention has been found to bio-resorb by 50% in approximately 100 days. In a study by Horan et al., Sprague-Dawley rats were used to compare the bio-resorption of embodiments according to the present invention to Mersilene™ mesh (Ethicon, Somerville, N.J.). The histology reports from the article state that after 94 days, 43% of the initial mesh of the embodiments remained compared to 96% of the Mersilene™ mesh. It was also reported that the in growth was more uniform with the mesh of embodiments than the Mersilene™ mesh. The Mersilene™ was found to have less in growth in the defect region than along the abdominal wall.

Physical properties include thickness, density and pore sizes. The thickness was measured utilizing a J100 Kafer Dial Thickness Gauge. A Mitutoyo Digimatic Caliper was used to find the length and width of the samples; used to calculate the density. The density was found by multiplying the length, width and thickness of the mesh then dividing the resulting value by the mass. The pore size was found by photographing the mesh with an Olympus SZX7 Dissection Microscope under 0.8× magnification. The measurements were taken using ImagePro 5.1 software and the values were averaged over several measurements. The physical characteristics of the sample meshes, including embodiments according to the present invention, are provided in TABLE 2.

TABLE 2

| | Physical Characterization | | |
|---|---|---|---|
| Device | Thickness (mm) | Pore Size (mm²) | Density (g/cm³) |
| Mersilene Mesh | 0.31 ± 0.01 | 0.506 ± 0.035 | 0.143 ± 0.003 |
| Bard Mesh | 0.72 ± 0.00 | 0.465 ± 0.029 | 0.130 ± 0.005 |
| Vicryl Knitted Mesh | 0.22 ± 0.01 | 0.064 ± 0.017 | 0.253 ± 0.014 |
| Present Embodiments - | 1.0 ± 0.04 | 0.640 ± 0.409 | 0.176 ± 0.002 |
| Single Needle Bed | | | |
| (SB) | | | |
| Present Embodiments - | 0.80 ± 0.20 | 1.27 | 0.135-0.165 |
| Double Needle Bed | | | |
| (DB) | | | |

All devices were cut to the dimensions specified in TABLE 3, for each type of mechanical analysis. Samples were incubated in phosphate buffered saline (PBS) for 3±1.25 hours at 37±2° C. prior to mechanical analysis to provide characteristics in a wet environment. Samples were removed from solution and immediately tested.

TABLE 3

| Test Modality | Length (mm) | Width (mm) |
|---|---|---|
| Tensile | 60 | 10 |
| Burst | 32 | 32 |
| Suture Pull-Out | 40 | 20 |
| Tear | 60 | 40 |
| Tensile Fatigue | 60 | 40 |

Ball burst test samples were scaled down due to limitations in material dimensions. The test fixture employed was a scaled (1:2.5) version of that recommended by ASTM Standard D3787. The samples were centered within a fixture and burst with a 10 mm diameter ball traveling at a displacement rate of 60 mm/min. Maximum stress and stiffness were determined from the burst test. Results can be seen in TABLE 4.

TABLE 4

Burst Strength

| Device | Stress (MPa) | Stiffness (N/mm) |
|---|---|---|
| Mersilene Mesh | 0.27 ± 0.01 | 13.36 ± 0.85 |
| Bard Mesh | 0.98 ± 0.04 | 38.28 ± 1.49 |
| Vicryl Knitted Mesh | 0.59 ± 0.05 | 32.27 ± 1.86 |
| Pelvitex Polypropylene Mesh | 0.59 ± 0.04 | 29.78 ± 1.33 |
| Permacol Biologic Implant | 1.27 ± 0.27 | 128.38 ± 22.14 |
| Present Embodiments (SB) | 0.76 ± 0.04 | 46.10 ± 2.16 |
| Present Embodiments (DB) | 0.66 | 40.9 |

Tensile tests were preformed along the fabric formation and width axes of each device. A 1 cm length of mesh on each end of the device was sandwiched between pieces of mm thick silicone sheet and mounted in pneumatic fabric clamps with a clamping pressure of 70-85 psi. Samples were loaded through displacement controlled testing at a strain rate of 100%/s (2400 mm/min) and or 67%/s (1600 mm/min) until failure. The ultimate tensile strength (UTS), linear stiffness and percent elongation at break can be seen in the following tables. Results can be found in TABLES 5-8. An entry of “NT” indicates that the data has not yet been tested.

TABLE 5

| | Tensile SPTF (Fabric Formation Axis—1600 mm/min) | | | |
|---|---|---|---|---|
| | Strength | Stress | Stiffness | % Elong. @ |
| Device | (N) | (MPa) | (N/mm) | Break |
| Mersilene Mesh | 46.14 ± 3.15 | 10.04 ± 0.71 | 0.90 ± 0.06 | 132.1% ± 9.3% |
| Bard Mesh | 30.90 ± 2.0 | 16.64 ± 1.16 | 3.32 ± 0.26 | 106.5% ± 3.2% |
| Vicryl Knitted | 35.69 ± 3.30 | 35.89 ± 4.48 | 2.59 ± 0.33 | 89.0% ± 7.3% |
| Mesh | | | | |
| Present | 76.72 ± 4.36 | 10.06 ± 0.38 | 7.13 ± 0.50 | 41.5% ± 2.3% |
| Embodiments | | | | |
| (SB) | | | | |
| Present | NT | NT | NT | NT |
| Embodiments | | | | |
| Mesh(DB) | | | | |

TABLE 6

| | Tensile SPTF (Fabric Formation Axis—2400 mm/min) | | | |
|---|---|---|---|---|
| | Strength | Stress | Stiffness | % Elong. @ |
| Device | (N) | (MPa) | (N/mm) | Break |
| Mersilene Mesh | 43.87 ± 5.19 | 14.15 ± 1.68 | 2.18 ± 0.3 | 56.6% ± 3.5% |
| Bard Mesh | 35.29 ± 5.69 | 4.90 ± 0.79 | 0.80 ± 0.23 | 177.3% ± 13.2% |
| Vicryl Knitted | 30.88 ± 3.30 | 14.04 ± 1.50 | 0.76 ± 0.17 | 191.9% ± 14.2% |
| Mesh | | | | |
| Pelvite | 23.05 ± 3.75 | 5.36 ± 0.87 | 0.57 ± 0.07 | 110.0% ± 13.6% |
| Polypropylene | | | | |
| Mesh | | | | |
| Permacol | 164.52 ± 30.58 | 13.71 ± 2.55 | 23.94 ± 2.7 | 23.5% ± 3.3% |
| Biologic Implant | | | | |
| Present | 72.31 ± 7.80 | 6.95 ± 0.75 | 4.31 ± 0.3 | 45.5% ± 5.2% |
| Embodiments | | | | |
| (SB) | | | | |
| Present | 74.62 ± 2.70 | 8.68 ± 0.31 | 4.25 ± 0.13 | 48.3% ± 2.1% |
| Embodiments | | | | |
| (DB) | | | | |

TABLE 7

| | Tensile SPTF (Fabric Width Axis—2400 mm/min) | | | |
|---|---|---|---|---|
| | Strength | Stress | Stiffness | % Elong. @ |
| Device | (N) | (MPa) | (N/mm) | Break |
| Mersilene Mesh | 31.14 ± 2.21 | 10.04 ± 0.71 | 0.90 ± 0.06 | 132.1% ± 9.3% |
| Bard Mesh | 119.80 ± 8.36 | 16.64 ± 1.16 | 3.32 ± 0.26 | 106.5% ± 3.2% |
| Vicryl Knitted | 78.96 ± 9.86 | 35.89 ± 4.48 | 2.59 ± 0.33 | 89.0% ± 7.3% |
| Mesh | | | | |
| Present | 104.58 ± 3.96 | 10.06 ± 0.38 | 7.13 ± 0.50 | 41.5% ± 2.3% |
| Embodiments | | | | |
| (SB) | | | | |
| Present | NT | NT | NT | NT |
| Embodiments | | | | |
| (DB) | | | | |

TABLE 8

| | Tensile SPTF (Fabric Width Axis—2400 mm/min) | | | |
|---|---|---|---|---|
| | Strength | Stress | Stiffness | % Elong. @ |
| Device | (N) | (MPa) | (N/mm) | Break |
| Mersilene Mesh | 28.11 ± 2.93 | 28.11 ± 2.93 | 1.05 ± 0.13 | 128.2% ± 23.6% |
| Bard Mesh | 103.53 ± 8.92 | 14.38 ± 1.24 | 3.43 ± 0.5 | 94.0% ± 8.4% |
| Vicryl Knitted | 106.65 ± 8.46 | 48.48 ± 3.85 | 5.08 ± 0.1 | 58.6% ± 8.4% |
| Mesh | | | | |
| Pelvite | 30.24 ± 5.77 | 7.03 ± 1.34 | 1.48 ± 0.1 | 89.6% ± 9.6% |
| Polypropylene | | | | |
| Mesh | | | | |
| Permacol | 67.71 ± 13.36 | 5.64 ± 1.11 | 8.56 ± 2.0 | 27.4% ± 4.2% |
| Biologic Implant | | | | |
| Present | 98.84 ± 4.79 | 9.50 ± 0.46 | 8.48 ± 0.3 | 39.0% ± 4.1% |
| Embodiments | | | | |
| (SB) | | | | |
| Present | 70.08 ± 2.55 | 8.15 ± 0.30 | 5.87 ± 0.22 | 33.6% ± 2.0% |
| Embodiments | | | | |
| (DB) | | | | |

Tear Strength was found through a method that entailed cutting a 10 mm “tear” into the edge, perpendicular to the long axis edge and centered along the length of the mesh. The mesh was mounted in pneumatic fabric clamps as previously described in the tensile testing methods. Samples were loaded through displacement controlled testing at a strain rate of 100%/s (2400 mm/min) until failure. The load at failure and the mode of failure are shown in TABLE 9.

TABLE 9

| | Tear Strength | |
|---|---|---|
| Device | Strength (N) | Failure Mode |
| Mersilene Mesh | 110.30 ± 5.63 | Tear Failure: 6/6 |
| Bard Mesh | 181.70 ± 12.33 | Tear Failure: 6/6 |
| Vicryl Knitted Mesh | 109.35 ± 4.85 | Tear Failure: 6/6 |
| Pelvitex Polypropylene Mesh | 108.14 ± 6.95 | Tear Failure: 4/6 |
| Permacol Biologic Implant | 273.79 ± 65.57 | Tear Failure: 6/6 |
| Embodiments (SB) | 194.81 ± 9.12 | Tear Failure: 6/6 |
| Embodiments (DB) | NT | NT |

Tensile fatigue testing was preformed on the surgical mesh device according to aspects of the present invention and representative predicate types including Vicryl Mesh and Bard Mesh. Samples were loaded into the pneumatic fabric clamps as previously described in the tensile testing methods above. Samples were submerged in PBS at room temperature during cycling. Sinusoidal load controlled cycling was preformed to 60% of mesh ultimate tensile strength. Number of cycles to failure was determined during the cyclic studies and can be seen in TABLE 10, where failure was indicated by fracture or permanent deformation in excess of 200%.

TABLE 10

| | Tensile Fatigue |
|---|---|
| Device | Cycles, 60% UTS |
| Bard Mesh | 6994 ± 2987 |
| Vicryl Knitted Mesh | 91 ± 127 |
| Embodiments (DB) | 1950 ± 1409 |

A method was developed to compare the suture pull out strength of the surgical mesh device according to aspects of the present invention to other surgical mesh on the market. Tested mesh was sutured with three 3.5 mm diameter suture anchors (Arthrex, Naples, Fla.) and secured to 15 pcf solid rigid polyurethane foam. Each device was positioned with the center of the 20 mm width over the center anchor with a 3 mm suture bite distance employed during suturing of the mesh to the 3 anchors. The saw bone was mounted in the lower pneumatic fabric clamp and offset to provide loading along the axis of the device when the device was centered under the load cell. The free end of the mesh was sandwiched between the silicone pieces and placed in the upper fabric clamp with 85±5 psi clamping force. Testing was preformed under displacement control with a strain rate of 100%/s (1620 mm/min). Maximum load at break and failure mode can be seen in TABLE 11.

TABLE 11

| | Suture-Pull-Out | |
|---|---|---|
| Device | Strength/Suture [N] | Failure Mode |
| Mersilene Mesh | 13.50 ± 1.65 | Mesh Failure: 6 of 6 |
| Bard Mesh | 28.80 ± 3.39 | Mesh Failure: 6 of 6 |
| Vicryl Knitted Mesh | 12.90 ± 1.30 | Mesh Failure: 6 of 6 |
| Pelvitex Polyproplene Mesh | 18.29 ± 4.04 | Mesh Failure: 6 of 6 |
| Permacol Biologic Implant | 47.36 ± 7.94 | Mesh Failure: 6 of 6 |
| Embodiments (SB) | 41.00 ± 2.98 | Mesh Failure: 6 of 6 |
| Embodiments (DB) | 32.57 ± 2.30 | Mesh Failure: 6 of 6 |

By utilizing the pattern for the double needle bed mesh and modifying the yarn size, yarn feed rate and/or needle bed width, the surgical mesh device according to aspects of the present invention would meet the physical and mechanical properties necessary for a soft or hard tissue repair depending on the application. Such properties include pore size, thickness, ultimate tensile strength, stiffness, burst strength and suture pull out. The pore size could be modified dependent to the feed rate to create a more open fabric and the thickness could range from 0.40 mm up to as wide as 19.0 mm. With modifications to the pore size and thickness the UTS, stiffness, burst strength and suture pull out would all be modified as well, most likely tailoring the modifications of the pore size and/or thickness to meet certain mechanical needs.

This mesh, created on the flat knitting machine would be made in such a way to increase or decrease pore size and/or thickness by changing the yarn size and/or changing the loop length found within the knitting settings. The loop placements in combination with the node lock design allow changes to the shape and/or to the mechanical properties of the mesh. A biocompatible yarn with elasticity, such as highly twisted silk, could be used for shaping.

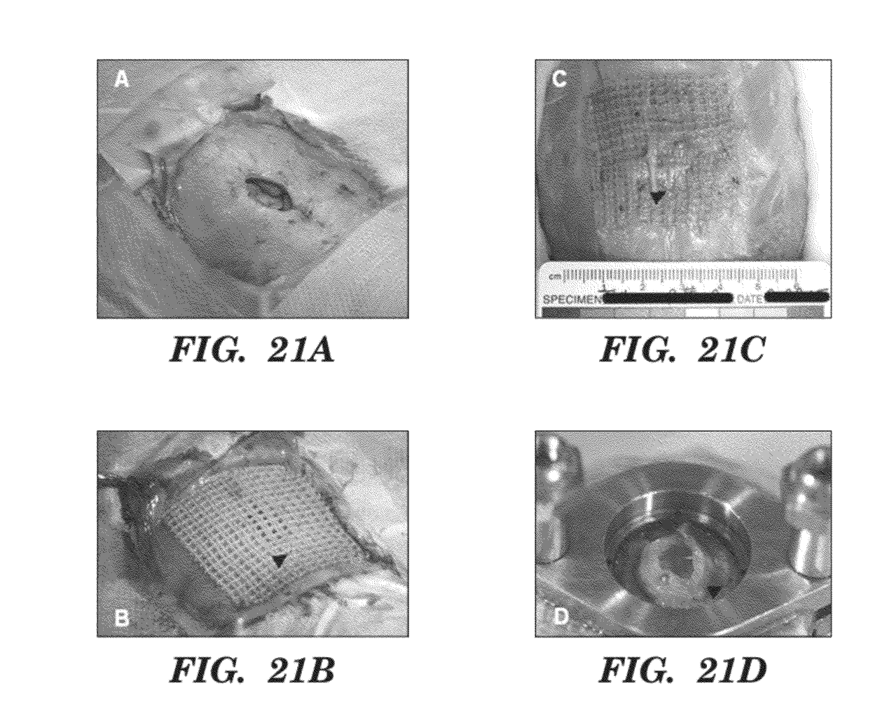

The implantation of a mesh and subsequent testing according to aspects of the present invention is illustrated in FIGS. 21A-D. FIG. 21A illustrates a full-thickness rat abdominal defect created using a custom designed 1-cm stainless steel punch. The defect appears oval in shape due to body wall tension applied. FIG. 21B illustrates a 4 cm×4 cm implant centered on top of the open defect, and held in place with single interrupted polypropylene sutures (arrow) through the implant and muscle. FIG. 21C illustrates an explanted specimen 94 days post implantation. FIG. 21D illustrates ball burst testing performed with a 1-cm diameter ball pushed through the defect site reinforced with the mesh.

While the present invention has been described in connection with a number of exemplary embodiments, and implementations, the present inventions are not so limited, but rather cover various modifications, and equivalent arrangements. For example, a knitted mesh according to aspects of the present invention may be used for a filler material. In one application, the knitted mesh may be cut into 1 mm×1 mm sections to separate one or more nodes, e.g., 3 nodes. The sections may be added to fat tissue or a hydro-gel to form a solution that can be injected into a defective area. Advantageously, the filler material may provide a desired texture, but will not unravel.

A surgical mesh in accordance with various aspects of the present invention is suitable for use in methods for body aesthetics and body contouring. Body aesthetics and body contouring surgical procedures include, but are not limited to, body contour abdominoplasty, tummy tuck, facial reconstruction, face lift, breast reconstruction, breast augmentation, mastopexy, pelvic floor repair, and hernia repair. In accordance with aspects of the present invention, a surgical mesh or scaffold is used, for example, to reinforce the abdominal wall during an abdominoplasty or tummy tuck procedure. A surgical mesh in accordance with aspects of the present invention is suitable for use in the event of massive weight loss which may coincide with weakness of the abdominal fascia to the point that the muscle stretches easily, or after a hernia recurrence following a previous abdominoplasty procedure. Existing procedures to reinforce the abdominal fascia involve use of an overlay with a non-resorbable mesh. In contrast, the methods of the present invention use a bioresorbable mesh that has characteristics that are capable of supporting the stress inflicted on the abdominal wall while providing a scaffold for regenerating tissue to support the abdominal wall stress once the mesh is completely bioresorbed. FIG. 100 illustrates the human anatomy in the abdominal area. In particular, FIG. 100 illustrates the surgical scaffold or mesh 10 applied over the abdominal area 12.

Although a variety of polymer materials may be employed, silk is a preferred biomaterial in the present invention. Particular embodiments may be formed from Bombyx Mori silkworm silk fibroin. The raw silk fibers have a natural globular protein coating known as sericin, which may have antigenic properties and must be depleted before implantation. Accordingly, the yarn is taken through a depletion process. The depletion of sericin is further described, for example, by Gregory H. Altman et al., “Silk matrix for tissue engineered anterior cruciate ligaments,” Biomaterials 23 (2002), pp. 4131-4141, the contents of which are incorporated herein by reference. As a result, the silk material used contains substantially no sensitizing agents, in so far as can be measured or predicted with standardized biomaterial test methods.

The surgical mesh in accordance with the present invention may be used in various dimensions depending upon factors including, but not limited to, the method or surgical procedure, the anatomy of the patient, among other factors. However, it is desirable for a surgical mesh to have dimensions ranging from 50 mm to 500 mm in width, from 50 mm to 500 mm in length and a thickness from a 0.4 mm to 4 mm Table 12 sets forth these ranges.

TABLE 12

| | Minimum (mm) | Maximum (mm) |
|---|---|---|
| Width | 50 | 500 |
| Length | 50 | 500 |
| Thickness | 0.4 | 4 |

An example of a suitable mesh size for use in body contour abdominoplasty is 30 cm×40 cm. An example of a suitable mesh design for use in body contour abdominoplasty is illustrated in FIGS. 34A-39. An example of a suitable mesh size for use in a tummy tuck procedure is 5 cm×40 cm. An example of a suitable mesh design for use in a tummy tuck is illustrated in FIGS. 50A-54. An example of a suitable mesh size for use in facial reconstruction and/or a face lift is 5 cm×5 cm. Examples of suitable mesh designs for use in facial reconstruction and/or a face lift are illustrated in FIGS. 28A-28J, in FIGS. 34A-39, and in FIGS. 50A-54. An example of a suitable mesh size for pelvic floor repair is 10 cm×12 cm. An example of a suitable mesh design for use in pelvic floor repair is illustrated in FIGS. 50A-53D. An example of a suitable mesh size for hernia repair is 30 cm×40 cm. An example of a suitable mesh design for use in complex hernia repair is illustrated in FIGS. 28A-28J.

Advantageously, the surgical mesh of the present invention has areas with different mechanical properties such as strength, stiffness and stretch. In the case of an abdominoplasty, it provides additional support to the different abdominal wall regions. The abdominal wall is formed by two layers of rectus fascia, also frequently referred to as the anterior sheath and the posterior sheath. The anterior sheath covers the full vertical dimension of the anterior abdomen. The posterior sheath covers the full vertical dimension of the anterior abdomen extending from the superior extent of the abdomen just below the ribs, and stopping as a transverse line. Typically, this line is a few centimeters below the umbilicus. This line where the posterior sheath stops is known as the arcuate line. The lack of a second layer of fascia in the lower abdomen results in most of the laxity observed below the arcuate line where only one layer of fascia exists. Therefore, a mesh having two areas, one with an increase in one or more mechanical properties is beneficial to support the single layer fascia in the lower abdomen area, while a second area with different mechanical properties in the upper abdomen area provides support to the double layer fascia. These methods are suitable, for example, in a post-pregnancy abdominoplasty or tummy tuck. FIG. 101 represents a surgical scaffold with two distinct areas, a mesh area 20 having lower properties and a mesh area 22 having higher properties.

The surgical mesh is formable into various shapes. For example, the surgical mesh may be shaped into a rectangle having four angles of equal size as illustrated in FIG. 102A, or it may be shaped into a trapezoid as illustrated in FIG. 103A, or it may be shaped as an hexagon as in FIG. 105A; however, the shapes illustrated are merely examples and the surgical mesh is not limited to any particular shape.

Advantageously, the surgical mesh may incorporate an opening along a centerline of the mesh. A surgical mesh having a round opening shape in the center of the mesh is illustrated in FIG. 102B. A surgical mesh having a rectangular opening shape in the center of the mesh is illustrated in FIG. 103B. A surgical mesh having an elliptical opening shape in the center of the mesh is illustrated in FIG. 103C. A slot with an opening, such as any of the above openings, in the center of the surgical mesh as represented in FIG. 104 allows for the placement of the mesh around the umbilicus without any additional shaping from the surgeon.

Providing a surgical mesh with a tailored opening around the umbilicus, will allow the correct placement of the mesh preventing the common healing complication reported when extra care is not taken with sutures and mesh in this location. The surgical mesh may be manufactured by using a warp knit construction when the surgeon desires the freedom for tailoring the mesh.

The surgical mesh may be made in various sizes to fit the patient population anatomy. For example, FIGS. 105A, B, and C represent three surgical meshes respectively in small, medium and large sizes.

The surgical mesh may be manufactured by using a weft knit construction when the surgeon desires a mesh available in multiple sizes already tailored for body contour without the need for cutting.

Both types of surgical mesh construction, warp and weft, allow for easy suturing with common size sutures used to secure the mesh to the abdominal wall. By piercing the mesh with a suture needle no damage is inflicted to the mesh structure.

The present invention relates to methods of using a silk surgical mesh or scaffold in accordance with aspects of the present invention in body aesthetics and body contouring. One such embodiment relates to use of the silk surgical mesh or scaffold in accordance with aspects of the present invention in abdominoplasty. There are various surgical procedures for performing an abdominoplasty depending upon the type of abdominoplasty to be performed. The time needed for conducting an abdominoplasty also varies depending upon the type of abdominoplasty to be performed. For example, a complete abdominoplasty typically is completed in 1 to 5 hours. A partial abdominoplasty, also referred to as a mini-tuck abdominoplasty, is typically completed in 1 to 2 hours. Following an abdominoplasty surgical procedure, reconstruction of the umbilicus, commonly referred to as the belly button, may also occur. The original umbilicus is attached, such as by sutures, into a new hole created by the surgeon.

A complete abdominoplasty is also referred to as a full abdominopasty. In a complete abdominoplasty, an incision is made from hip to hip just above the pubic area. Another incision is made to separate the navel from the surrounding skin. The skin is detached from the abdominal wall to reveal the muscles and fascia to be tightened. The muscle fascia wall is tightened with sutures. The remaining skin and fat are tightened by removing the excess and closing. The old belly button stalk is brought out through a new hole and sutured into place. Liposuction may also be used to refine the transition zones of the abdominal contouring. A surgical dressing and optionally a compression garment are applied. Excess fluid from the site is drained. A complete abdominoplasty may also comprise a musculofascial plication abdominal dermal lipectomy and/or suction-assisted lipectomy of hips.

A partial abdominoplasty is also referred to as a mini abdominoplasty. In a partial abdominoplasty, a smaller incision is made as compared to a complete abdominoplasty. The skin and fat of the lower abdomen are detached in a more limited manner from the muscle fascia. The skin is stretched down and excess skin removed. The belly button stalk may be divided from the muscle below and the belly button slid down lower on the abdominal wall.

A portion of the abdominal muscle fascia wall is optionally tightened. Liposuction is often used to contour or sculpt the transition zone. The flap is stitched back into place. A combination abdominoplasty and liposuction procedure is often referred to as a “lipo-tuck”. During such procedure, skin is removed and subsequently sutured. As noted above, the belly button is reattached to a new hole created by the surgeon.

An extended abdominoplasty is a complete abdominoplasty plus a lateral thigh lift. The patient is cut from the posterior axillary line. The operation includes all of the abdominal contouring of a complete abdominoplasty plus allows further improvement of the flank (waist), as well as smoothing the contour of the upper lateral thigh.

A high lateral tension tummy tuck is a more involved procedure and typically takes at least four and half hours to perform. In this method, in addition to vertical-line tightening as is the case in most conventional abdominoplasty procedures, muscles are also tightened horizontally. The procedure provides a patient with a flat abdomen and with an improved waistline.

A circumferential abdominoplasty, also referred to as a belt lipectomy or body lift, is an extended abdominoplasty in conjunction with a buttock lift. The incision typically runs all the way around the body. This surgical procedure is suitable, for example, for patients who have undergone massive weight loss.

The above procedures can be used alone or in combination. For example, an abdominoplasty may be conducted in the course of a lower body lift. Alternatively, abdominoplasty is combinable with liposuction contouring, breast reduction, breast lift, or a hysterectomy. Breast enhancement procedures performed in conjunction with an abdominoplasty are often referred to as a “mommy makeover”. In such a procedure, barbed sutures may be employed.

Another method of using a silk surgical mesh or scaffold in accordance with aspects of the present invention is for hernia repair. In general, there are two main types of hernia repair: open hernia repair and minimally invasive (laparoscopic) repair. Open repair is a traditional hernia repair procedure. There are numerous and varied approaches for performing this type of hernia repair. Such approaches are performed routinely with local and intravenous sedation. Due to the larger size of the incision, open hernia repair is generally painful with a relatively long recovery period. Minimally invasive (laparoscopic) repair is usually performed under general anesthesia. Spinal anesthesia and local anesthesia are used under certain circumstances. Benefits associated with minimally invasive (laparoscopic) repair include shorter operative time, less pain, and a shorter recovery period.

In laparoscopic hernia surgery, a telescope attached to a camera is inserted through a small incision that is made under the patient's belly button. Two other small cuts are made in the lower abdomen. The hernia defect is reinforced with a mesh and secured in position. The mesh is secured in position by stitches, staples, tacks, and glue.

Another form of laparoscopic hernia repair is ventral hernia repair (laparoscopic). Incisional, ventral, epigastric, or umbilical hernias are defects of the anterior abdominal wall and may be congenital (umbilical hernia) or acquired (incisional). Incisional hernias form after surgery through the incision site or previous drain sites, or laparoscopic trocar insertion sites. Incisional hernias often occur after open surgical procedures. These hernias present with a bulge near or at a previous incision. A prosthetic mesh is used in order to minimize tension on the repair so as to reduce the chance of hernia recurrence. Traditionally, an old incision scar is incised and removed. Inspection of the entire length of the incision generally uncovers multiple hernia defects. The area requiring coverage is usually large and requires much surgical dissection. A prosthetic mesh is used to cover the defect before closure of the wound. This is a major and often complex surgical procedure. The use of prosthetic mesh decreases possible recurrence. A patient typically returns to normal activity within a matter of weeks. The principles governing a laparoscopic ventral hernia repair are based on those of open Stoppa ventral hernia repair. A large piece of prosthetic mesh is placed under the hernia defect with a wide margin of mesh outside the defect, and the mesh is anchored in to place and secured to the anterior abdominal wall. The mesh is anchored into place, for example, by sutures. The mesh is secured to the anterior abdominal wall, for example, by tacks which are placed laparoscopically.

### Example

An abdominoplasty procedure was conducted using a silk based node-lock mesh having the mesh or scaffold design shown in FIGS. 28A-28J and having the dimensions of 10 cm×25 cm. The typical fascial “work” was done first, using a row of figure of eight sutures, first and then another layer of running suture all #1 PDS (polydioxanone suture, Ethicon) Two pieces of silk based node-lock mesh as described above were used as an “onlay” to augment the fascia “tightening”. One 10×25 cm piece was used in the lower abdomen. It was placed transversely, the vertical dimension 10 cm, was positioned with the lower edge at level of the pubic symphsis, and the upper edge at the lower border of the umbilicus. The 25 cm transverse dimension, was suitable. A second scaffold was cut and tailored to use in the supra-umbilcal region, with care taken not to leave too close to the umbilical closure. As the closure around the umbilicus occasionally may not heal primarily, extra care was taken with sutures/mesh in this location.

The handling characteristics were excellent. The scaffold was secured at its periphery with a 3-0 V-Lock (COVIDIEN brand of barbed suture, made of a material similar to PDS).

The use of the scaffold added about 15 minutes to the case. It was estimated that it could take between 5 to 10 minutes in a subsequent procedure. The entire case was about 7 and a half hours, for both liposuction and the body lift.

The patient did very well and was hospitalized overnight. The patient had a total of 5, ten mm flat blake drains—two that drain the back and three in the front. The patient was to stay on antibiotics until drains were which is typically within 10 to 20 days.

An embodiment of the present invention is surgical scaffold (a medical device) prepared as a knitted, multi-filament, bioengineered silk. Preferably this silk surgical scaffold is mechanically strong, biocompatible, and long-term bioresorbable. This silk surgical scaffold can be a sterile, single use only medical device, supplied in different sizes and ready for use in open or laparoscopic procedures. The silk surgical scaffold is flexible and well suited for delivery through a laparoscopic trocar due to its strength and flexibility, and offers tear resistance, suture retention, and ability to be cut in any direction. The silk surgical scaffold provides immediate physical and mechanical stabilization of a tissue defect through its strength and porous (scaffold-like) construction. A proteolytic digestion study showed that the mechanical strength of a preferred silk surgical scaffold decreases by 50% thirty days post-implantation with a corresponding 25% decrease in mass. The proteolytic digestion study was designed to model bioresorption independent of contributing host tissue regenerated during the tissue repair response post-implantation. Actual residence time until complete bioresorption of the silk surgical scaffold can vary based on site of implantation and patient physiology, and can be as long as two years.

A preferred silk surgical scaffold can be used as a transitory scaffold for soft tissue support and repair to reinforce deficiencies where weakness or voids exist that require the addition of material to obtain the desired surgical outcome, including but not limited to reinforcement of soft tissues in reconstructive and plastic surgery to obtain the desired aesthetic outcome. The silk surgical scaffold should not be used in patients with a known allergy to silk nor in direct contact with bowel or viscera where formation of adhesions may occur. To use a preferred silk surgical scaffold:

1. irrigate and aspirate the device implant site with saline following the in situ cutting of the device to remove any device particulate debris that may have been generated.

2. a preferred silk surgical scaffold should be stored in its original sealed package away from direct sources of heat at ambient room temperature.

3. handle a preferred silk surgical scaffold using aseptic technique and sterile talc-free gloves.

4. remove a preferred silk surgical scaffold device from the package. Although a preferred silk surgical scaffold does not require rehydration for mechanical or physical performance, a brief incubation (minimum 2-3 seconds) in sterile rinse solution is recommended prior to implantation.

5. use the type of suture or fixation system that is appropriate for the patient use.

6. sutures should be placed at least 3 mm, or one full row, from the cut edge of the preferred silk surgical scaffold.

7. If preferred, the uncut preferred silk surgical scaffold device may be sutured over the patient defect and trimmed once secured in place followed by rinsing and aspiration.

8. the preferred silk surgical scaffold device should be sufficiently anchored to stabilize it during tissue ingrowth.

9. for laparoscopic procedures the preferred silk surgical scaffold should be rolled along its long axis and may be delivered through a ⅞ mm or larger cannula.

What is claimed is:

1. A method of using a silk surgical mesh, the method comprising:
providing a silk surgical mesh, wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes, and
applying the silk surgical mesh in an abdominal area of a patient, wherein the abdominal area comprises an upper abdominal area and a lower abdominal area.

2. The method of using according to claim 1, wherein the silk surgical mesh is applied in the lower abdominal area beneath an arcuate line.

3. The method of using according to claim 1, wherein the silk surgical mesh is bioresorbable and capable of supporting stress inflicted on an abdominal wall in the abdominal area of the patient.

4. The method of using according to claim 1, wherein the silk surgical mesh is 50 mm to 500 mm in width.

5. The method of using according to claim 1, wherein the silk surgical mesh is 50 mm to 500 mm in length.

6. The method of using according to claim 1, wherein the silk surgical mesh is 0.4 mm to 4 mm in thickness.

7. The method of using according to claim 1, wherein the silk is formed from Bombyx mori silkworm silk fibroin.

8. The method of using according to claim 1, wherein the silk is sericin depleted.

9. The method of using according to claim 1, wherein the silk surgical mesh is applied in the lower abdominal area and in the upper abdominal area.

10. The method of using according to claim 1, wherein the mesh has at least two areas that differ in at least one mechanical property.

11. The method of using according to claim 10, wherein the first area supports a single layer fascia in the lower abdomen and the second area supports a double layer fascia in the upper abdomen.

12. The method of using according to claim 10, wherein the mechanical property is strength, stiffness or stretch.

13. The method of using according to claim 10, wherein the first area has a greater strength, stiffness, or stretch than the second area.

14. The method of using according to claim 1, wherein the silk surgical mesh is in a shape of a circle, rectangle, square, ellipse, trapezoid, hexagon, or combination thereof.

15. The method of using according to claim 1, wherein the silk surgical mesh comprises an opening along a centerline of the silk surgical mesh.

16. The method of using according to claim 1, wherein the opening is in a shape of a circle, rectangle, square, ellipse, trapezoid, or hexagon.

17. The method of using according to claim 15, wherein the mesh further comprises a slit with the opening along the centerline of the mesh to provide for placement of the silk surgical mesh around an umbilicus.

18. The method of using according to claim 1, wherein the silk surgical mesh has a weft or warp knit construction.

19. The method of using according to claim 1, wherein the method further comprises suturing the silk surgical mesh to the abdominal area.

20. A method of using a silk surgical mesh in an abdominoplasty or a tummy tuck surgical procedure, the method comprising:
providing a silk surgical mesh having an opening, and
placing the silk surgical mesh around an umbilicus of a patient, wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes.

21. A method of using a silk surgical mesh, the method comprising:
providing a silk surgical mesh, and
applying the silk surgical mesh in a facial area of a patient wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes.

22. A method of using a silk surgical mesh in a pelvic floor repair, the method comprising:
providing a silk surgical mesh, and
applying the silk surgical mesh in a pelvic area of a patient wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes.

23. A method of using a silk surgical mesh in hernia repair, the method comprising:
providing a silk surgical mesh, and
applying the silk surgical mesh in an abdominal area of a patient wherein the silk surgical mesh is a knitted mesh including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes.

24. A method for using a knitted silk fabric in a surgical procedure, the method comprising the steps of:
(a) providing a biodegradable, knitted silk fabric, and;
(b) placing the biodegradable, knitted silk fabric in or over an abdominal area of a patient.

25. The method of claim 24, wherein a first silk fabric with an upper edge and a lower edge is placed in the lower abdomen.

26. The method of claim 25 wherein the first silk fabric is positioned with a lower edge at the level of the pubic symphsis, and the upper edge at the lower border of the umbilicus.

27. The method of claim 24 wherein a second silk fabric with an upper edge and a lower edge is placed in the lower abdomen.

28. The method of claim 27, wherein the second silk fabric is placed in the supra-umbilcal region.

29. The method of claim 24 wherein the silk fabric is a non-woven, waft (vertical) knit, multi-filament silk fabric.
